SEQUENCE LISTING (202 sequences)

SEQ ID NO: 1 - DNA, 368 bp, Homo sapiens
gaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacaggccgtccttccaaggccaggtcaccatctcagccgacaagtccatcaccaccgcctacctgcagtggagcagcctgaaggcctcggacaccgccatgtattactgtgcgagacacccttcctatggttcggggagtcccaactttgactactggggccagggaaccctggtcaccgtctcctcagc

SEQ ID NO: 2 - protein, 122 aa, Homo sapiens
EVQLVQSGAEVKKPGESLKISCKGSGYSFTSYWIGWVRQMPGKGLEWMGIIYPGDSDTRYRPSFQGQVTISADKSITTAYLQWSSLKASDTAMYYCARHPSYGSGSPNFDYWGQGTLVTVSS

SEQ ID NO: 3 - DNA, 427 bp, Homo sapiens
gatattgtgatgacccagactccactctcctcacctgtcacccttggacagccggcctccatctcctgcaggtctagtcaaagcctcgtacacagtgatggaaacacctacttgagttggcttcagcagaggccaggccagcctccaagactcctaatttataagatttctaaccggttctctggggtcccagacagattcagtggcagtggggcagggacagatttcacactgaaaatcagcagggtggaagctgaggatgtcggggtttattactgcatgcaagctatacaatttccgatcaccttcggccaagggacacgactggagattaaacgaactgtggctgcaccatctgtcttcatcttcccgccatctgatgagcagttgaaatctggaactgcctctgttgtgtgcctgctgaata

SEQ ID NO: 4 - protein, 112 aa, Homo sapiens
DIVMTQTPLSSPVTLGQPASISCRSSQSLVYSDGNTYLSWLQQRPGQPPRLLIYKISNRFSGVPDRFSGSGAGTDFTLKISRVEAEDVGVYYCVQATQFPLTFGGGTKVEIK

SEQ ID NO: 5 - DNA, 374 bp, Homo sapiens
gaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagaatgggggatttttggagtggttattataccagaggtatggacgtctggggccaagggaccacggtcaccgtctcctcagc

SEQ ID NO: 6 - protein, 124 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARMGDFWSGYYTRGMDVWGQGTTVTVSS

SEQ ID NO: 7 - DNA, 323 bp, Homo sapiens
gaaatagtgatgacgcagtctccagccaccctgtctgtgtctccaggggaaagagccaccctctcctgcagggccagtcagagtgttagcagcaacttagcctggtaccagcagaaacctggccaggctcccagactcctcatctctggtgcatccaccagggccactggtatcccagccaggttcagtggcagtgggtctgggacagagttcactctcaccatcagcagcctgcagtctgaagattttgcagtttattactgtcaccagtataatgactggtctctcactttcggcggagggaccaaggtggagatcaaacg

SEQ ID NO: 8 - protein, 107 aa, Homo sapiens
EIVMTQSPATLSVSPGERATLSCRASQSVSSNLAWYQQKPGQAPRLLISGASTRATGIPARFSGSGSGTEFTLTISSLQSEDFAVYYCHQYNDWSLTFGGGTKVEIK

SEQ ID NO: 9 - DNA, 365 bp, Homo sapiens
gaggtgcagctggtgcagtctggagcagaggtgaaaaagcccggggagtctctgaagatctcctgtaagggttctggatacagctttaccaactactggatcgtctgggtgcgccagatgcccgggaaaggcctggagtggatggggatcatctatcctggtgactctgataccagatacagcccgtccttccaaggccaggtcaccatctcagccgacaagtccatcagcaccgcgtacctgcagtggagcagcctgaaggcctcggacaccgccatgtactactgtgcgagacatggagattactactactactacggtatggacgtctggggccaagggaccacggtcaccgtctcctcagc

SEQ ID NO: 10 - protein, 121 aa, Homo sapiens
EVQLVQSGAEVKKPGESLKISCKGSGYSFTNYWIVWVRQMPGKGLEWMGIIYPGDSDTRYSPSFQGQVTISADKSISTAYLQWSSLKASDTAMYYCARHGDYYYYYGMDVWGQGTTVTVSS

SEQ ID NO: 11 - DNA, 338 bp, Homo sapiens
gatattgtgatgacccagaccccactctcctcacctgtcacccttggacagccggcctccatctcctgcaggtctagtcaacgcctcgtacacagtgatggacacacctatttgagttggcttcagcagaggccaggccagcctccaagactcctaatttctaaggtttctaaccggttctctggggtcccagacagattcagtggcagtggggcagggacagatttcacactgaaaatcagcagggtggaagctgaggatgtcgggatttattactgcatgcaagctacacaatttccgctcactttcggcggagggaccaaggtggagatcaaacg

SEQ ID NO: 12 - protein, 112 aa, Homo sapiens
DIVMTQTPLSSPVTLGQPASISCRSSQRLVHSDGHTYLSWLQQRPGQPPRLLISKVSNRFSGVPDRFSGSGAGTDFTLKISRVEAEDVGIYYCMQATQFPLTFGGGTKVEIK

SEQ ID NO: 13 - DNA, 368 bp, Homo sapiens
gaggtgcagctggtgcagtctggagcagaggtgaaaaagcccggggagtctctgaagatctcctgtaagggttctggatacagctttaccagctactggatcggctgggtgcgccagatgtccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatatattactgtgcgagacacccttcctatggttcggggagtcccaactttgactactggggccagggaaccctggtcaccgtctcctcagc

SEQ ID NO: 14 - protein, 122 aa, Homo sapiens
EVQLVQSGAEVKKPGESLKISCKGSGYSFTSYWIGWVRQMSGKGLEWMGIIYPGDSDTRYSPSFQGQVTISADKSISTAYLQWSSLKASDTAIYYCARHPSYGSGSPNFDYWGQGTLVTVSS

SEQ ID NO: 15 - DNA, 338 bp, Homo sapiens
gatattgtgatgacccagactcctctctcctcacctgtcacccttggacagccggcctccatctcctgcaggtctagtcaaagcctcgtatacagtgatggaaacacctacttgagttggcttcagcagaggccaggccagcctccaagactcctattttataagatttctaatcggttctctggggtcccagacagattcagtggcagtggggcagggacagatttcacactgaaaatcagcagggtggaagctgaggatgtcggggtttattactgcgtgcaagctacacaatttcctctcactttcggcggagggaccaaggtggagatcaaacg

SEQ ID NO: 16 - protein, 112 aa, Homo sapiens
DIVMTQTPLSSPVTLGQPASISCRSSQSLVYSDGNTYLSWLQQRPGQPPRLLFYKISNRFSGVPDRFSGSGAGTDFTLKISRVEAEDVGVYYCVQATQFPLTFGGGTKVEIK

SEQ ID NO: 17 - DNA, 365 bp, Homo sapiens
gaggtgcagctggtgcagtctggagcagaggtgaaaaagcccggggagtctctgaagatctcctgtaagggttctggatacagctttaccaactactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacaaggggattactaccactattccggtatggacgtctggggccaagggaccacggtcaccgtctcctcagc

SEQ ID NO: 18 - protein, 121 aa, Homo sapiens
EVQLVQSGAEVKKPGESLKISCKGSGYSFTNYWIGWVRQMPGKGLEWMGIIYPGDSDTRYSPSFQGQVTISADKSISTAYLQWSSLKASDTAMYYCARQGDYYHYSGMDVWGQGTTVTVSS

SEQ ID NO: 19 - DNA, 338 bp, Homo sapiens
gatgttgtgatgactcagtctccactctccttgtccgtcacccttggacagccggcctccatctcctgcaggtctagtcaaagcctcgtatacaatgatggagacacctacttgaattggtttcagcagaggccaggccaatctccaaggctcctaatttataaggtttctaactgggactctggggtccccgacagattcagcggcagtgggtcaggcactgatttcacactgaaagtcagcagggtggaagctgaggatgttggggtttattactgcatgcaaggtacacactggcctctcactttcggcggagggaccaaggtggagatcaaacg

SEQ ID NO: 20 - protein, 112 aa, Homo sapiens
DVVMTQSPLSLSVTLGQPASISCRSSQSLVYNDGDTYLNWFQQRPGQSPRLLIYKVSNWDSGVPDRFSGSGSGTDFTLKVSRVEAEDVGVYYCMQGTHWPLTFGGGTKVEIK

SEQ ID NO: 21 - DNA, 365 bp, Homo sapiens
gaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagactaggggattactataactactacggtatggacgtctggggccaagggaccacggtcaccgtctcctcagc

SEQ ID NO: 22 - protein, 121 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLGDYYNYYGMDVWGQGTTVTVSS

SEQ ID NO: 23 - DNA, 338 bp, Homo sapiens
gatattgtgatgacccagactccactctcctcacctgtcacccttggacagccggcctccatctcctgcaggtctagtcaaagcctcgtacacagtgatggaaaaacctacttgagttggcttcagcagaggccaggccagcctccaagactcctaatttataagagttctaaccggttctctggggtcccagacagattcagtggcagtggggcagggacagatttcacactgaaaatcagcagggtggaagctgaggatgtcggggtttattactgcgtgcaagctacacaatttcctatcaccttcggccaagggacacgactggagattaaacg

SEQ ID NO: 24 - protein, 112 aa, Homo sapiens
DIVMTQTPLSSPVTLGQPASISCRSSQSLVHSDGKTYLSWLQQRPGQPPRLLIYKSSNRFSGVPDRFSGSGAGTDFTLKISRVEAEDVGVYYCVQATQFPITFGQGTRLEIK

SEQ ID NO: 25 - DNA, 368 bp, Homo sapiens
gaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcaccaccgcctacctgcagtggagcagcctgaaggcctcggacaccgccatgtattactgtgcgagacacccttcctatggttcggggagtcccaactttgactactggggccagggaaccctggtcaccgtctcctcagc

SEQ ID NO: 26 - protein, 122 aa, Homo sapiens
EVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSS

SEQ ID NO: 27 - DNA, 326 bp, Homo sapiens
gaaattgtgttgacgcagtctccaggcaccctgtctttgtctccaggggaaagagccaccctctcctgcagggccagtcagagtgttagcagcagttacttagcctggtaccagcagaaacctggccaggctcccaggctcctcatctatggtgtatccagcagggccactggcatcccagacaggttcagtggcagtgggtctgggacagacttcactctcaccatcagcagactggagcctgaagattttgcagtgtgttactgtcagcactatggtacctcacctcggacgttcggccaagggaccaaggtggaaatcaaacg

SEQ ID NO: 28 - protein, 112 aa, Homo sapiens
DIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQAIQFPITFGQGTRLEIK

SEQ ID NO: 29 - DNA, 368 bp, Homo sapiens; identical to SEQ ID NO: 25

SEQ ID NO: 30 - protein, 122 aa, Homo sapiens; identical to SEQ ID NO: 26

SEQ ID NO: 31 - DNA, 338 bp, Homo sapiens
gatattgtgatgacccagactccactctcctcacctgtcacccttggacagccggcctccatctcctgcaggtctagtcaaagcctcgtatacagtgatggaaacacctacttgagttggcttcagcagaggccaggccagcctccaagactcctaatttataagatttctaaccggttctctggggtcccagacagattcagtggcagtggggcagggacagatttcacactgaaaatcagcagggtggaagctgaggatgtcggggtttattactgcgtgcaagctacacaatttcctctcactttcggcggagggaccaaggtggagatcaaacg

SEQ ID NO: 32 - protein, 112 aa, Homo sapiens; identical to SEQ ID NO: 4

SEQ ID NO: 33 - DNA, 365 bp, Homo sapiens
gaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacatggtgactacagtaacattgatgcttttgatatctggggccaagggacaatggtcaccgtctcttcagc

SEQ ID NO: 34 - protein, 121 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARHGDYSNIDAFDIWGQGTMVTVSS

SEQ ID NO: 35 - DNA, 332 bp, Homo sapiens
gatgttgtgatgactcagtctccactctccctgcccgtcacccttggacagccggcctccatctcctgcaggtctagtcaaagcctcgtatacagtgatggaaacgcctacttgaattggtttcagcagaggccaggccaatctccaaggcgcctaatttataaggtttctaactgggactctggggtcccagacagattcagcggcagtgggtcaggcactgatttcacactgaaaatcagaagggtggaggctgaggatgttggggcttattactgcatgcaaggtatacactgcactttcggccctgggaccaaagtgcatatcaaacg

SEQ ID NO: 36 - protein, 110 aa, Homo sapiens
DVVMTQSPLSLPVTLGQPASISCRSSQSLVYSDGNAYLNWFQQRPGQSPRRLIYKVSNWDSGVPDRFSGSGSGTDFTLKIRRVEAEDVGAYYCMQGIHCTFGPGTKVHIK

SEQ ID NO: 37 - DNA, 365 bp, Homo sapiens
gaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagaactggaactacggactactactccggtatggacgtctggggccaagggaccacggtcaccgtctcctcagc

SEQ ID NO: 38 - protein, 121 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARTGTTDYYSGMDVWGQGTTVTVSS

SEQ ID NO: 39 - DNA, 338 bp, Homo sapiens
gatattgtgatgacccagactccactctcctcacctgtcacccttggacagccggcctccatctcctgcaggtctagtcaaagcctcgtacacagtgatggaaacaccttcttgagttggcttcagcagaggccaggccagcctccaagactcctaatttataagatttctaaccggttctctggggtcccagacagattcagtggcagtggggcagggacagatttcacactgaaaatcagcagggtggaagctgaggatgtcggggtttattactgcatgcaacttactcaatttccgctcactttcggcggagggaccaaggtggagatcagacg

SEQ ID NO: 40 - protein, 112 aa, Homo sapiens
DIVMTQTPLSSPVTLGQPASISCRSSQSLVHSDGNTFLSWLQQRPGQPPRLLIYKISNRFSGVPDRFSGSGAGTDFTLKISRVEAEDVGVYYCMQLTQFPLTFGGGTKVEIR

SEQ ID NO: 41 - DNA, 368 bp, Homo sapiens
gaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccatccttccaaggccaggtcaccatctcagccgacaagtccatcagcaccgcctacctgcagtggagcagcctgaaggcctcggacaccgccatgtattactgtgcgagacatggggatttttggagtggttattattcttttgactactggggccagggaaccctggtcaccgtctcctcagc

SEQ ID NO: 42 - protein, 122 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARHGDFWSGYYSFDYWGQGTLVTVSS

SEQ ID NO: 43 - DNA, 341 bp, Homo sapiens
gatgttgtgatgactcagtctccactctccctgcccgtcacccttggacagccggcctccatctcctgcaggtctagtcaaagcctcgtatacagtgatggaaacacctacttgaattggtttcagcagaggccaggccaatctccaaggcgcctaatttataaggtttctaactgggactctggggtcccagacagattcagcggcagtgggtcaggcactgatttcacactgaaaatcagcagggtggaggctgaggatgttggggtttattactgcatgcaaggtacacactggccttcgatcaccttcggccaagggacacgactggagattaaacg

SEQ ID NO: 44 - protein, 113 aa, Homo sapiens
DVVMTQSPLSLPVTLGQPASISCRSSQSLVYSDGNTYLNWFQQRPGQSPRRLIYKVSNWDSGVPDRFSGSGSGTDFTLKISRVEAEDVGVYYCMQGTHWPSITFGQGTRLEIK

SEQ ID NO: 45 - DNA, 368 bp, Homo sapiens
gaggtgcagttggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacagggcgatttttggagtggttatgggggtatggacgtctggggccaagggaccacggtcaccgtctcctcagc

SEQ ID NO: 46 - protein, 122 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSS

SEQ ID NO: 47 - DNA, 338 bp, Homo sapiens
gatattgtgatgacccagactccacactcctcacctgtcacccttggacagccggcctccatatcctgcaggtctagtcaaagcctcgtatccagagatggaaacacctacttgagttggcttcagcagaggccaggccagcctccaagactcctaatttataagatttctaaccggttctctggggtcccaaacagattcagtggcagtggggcagggacagatttcacactgaaaatcagcagggtgaaagctgaggatgtcggggtttattactgcatgcaagctacacagtttcccctcaccttcggccaagggacacgactggagattaaacg

SEQ ID NO: 48 - protein, 112 aa, Homo sapiens
DIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIK

SEQ ID NO: 49 - DNA, 368 bp, Homo sapiens
gaggtgcagttggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccaaatacagcccgtccttccaaggccaggtcaccatctcagccgacaagtccatcagcaccgcctacctgcagtggagcagcctgaaggcctcggacaccgccatgtattactgtgcgagacagggcgatttctggagtggtaatgggggtatggacgtctggggccaagggaccacggtcaccgtctcctcagc

SEQ ID NO: 50 - protein, 122 aa, Homo sapiens
EVQLVQSGAEVKKPGESLKISCKGSGYSFTSYWIGWVRQMPGKGLEWMGIIYPGDSDTKYSPSFQGQVTISADKSISTAYLQWSSLKASDTAMYYCARQGDFWSGNGGMDVWGQGTTVTVSS

SEQ ID NO: 51 - DNA, 338 bp, Homo sapiens
gatattgtgatgacccagactccactctcctcacctgtcacccttggacagccggcctccatctcctgcaggtctagtcaaagcctcgtatccagagatggaaacacctacttgagttggcttcagcagaggccaggccagcctccaagactcctaatttataagatttctaaccggttctctggggtcccagacagattcagtggcagtggggcagggacagatttcacactgaaaatcagcagggtggaagttgaggatgtcggcgtttattactgcatgcaagctacacaatttcccatcaccttcggccaggggacacgactggagattaaacg

SEQ ID NO: 52 - protein, 112 aa, Homo sapiens
DIVMTQTPLSSPVTLGQPASISCRSSQSLVSRDGNTYLSWLQQRPGQPPRLLIYKISNRFSGVPDRFSGSGAGTDFTLKISRVEVEDVGVYYCMQATQFPITFGQGTRLEIK

SEQ ID NO: 53 - DNA, 371 bp, Homo sapiens
gaggtgcagctggtgcagtctggagcagaggtgaaaaagcccggggagtctctgaagatctcctgtaagggttctggatacagctttaccaactactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacatggtgattactatgcttcggagagttctgcttttgatatctggggccaagggacaatggtcaccgtctcttcagc

SEQ ID NO: 54 - protein, 123 aa, Homo sapiens
EVQLVQSGAEVKKPGESLKISCKGSGYSFTNYWIGWVRQMPGKGLEWMGIIYPGDSDTRYSPSFQGQVTISADKSISTAYLQWSSLKASDTAMYYCARHGDYYASESSAFDIWGQGTMVTVSS

SEQ ID NO: 55 - DNA, 338 bp, Homo sapiens
gatattgtgatgacccagactccactctcctcacctgtcacccttggacagccggcctccatctcctgcaggtctagtcaaagcctcgtacacagtgatggaaacaccttcttgagttggcttcagcagaggccaggccagcctccaagactcctaatttataagatttctaaccggttctctggggtcccagacagattcagtggcagtggggcagggacagatttcacactgaaaatcagcagggtggaagctgaggatgtcggggtttattactgcatgcaaggtacacaatttcctctcactttcggcggagggaccaaggtggagatcaaacg

SEQ ID NO: 56 - protein, 112 aa, Homo sapiens
DIVMTQTPLSSPVTLGQPASISCRSSQSLVHSDGNTFLSWLQQRPGQPPRLLIYKISNRFSGVPDRFSGSGAGTDFTLKISRVEAEDVGVYYCMQGTQFPLTFGGGTKVEIK

SEQ ID NO: 57 - DNA, 365 bp, Homo sapiens
gaggtgcagctggtgcagtctggagcagaggtgaaaaagcccggggagtctctgaagatctcctgtaagggttctggatacagctttaccagctactggatcgactgggtgcgccagatgcccgggaaaggcctggagtggatggggtccatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacagggagcttcggggtactactacggtatggacgtctggggccaagggaccacggtcaccgtctcctcagc

SEQ ID NO: 58 - protein, 121 aa, Homo sapiens
EVQLVQSGAEVKKPGESLKISCKGSGYSFTSYWIDWVRQMPGKGLEWMGSIYPGDSDTRYSPSFQGQVTISADKSISTAYLQWSSLKASDTAMYYCARQGASGYYYGMDVWGQGTTVTVSS

SEQ ID NO: 59 - DNA, 338 bp, Homo sapiens
gatattgtgatgacccagagtccactctcctcacctgtcacccttggacagccggcctccatctcctgcaggtatagtcaaagcctcgtacacagggatggaaacacctacttgagttggcttcagcagaggccaggccagcctccaagactcgtaattcataagatttctaaccggttctctggggtcccagacagattcagtggcagtggggcagggacagatttcacactgaaaatcagcagggtggaagctgaggatgtcgggttttattactgcatgcaaactacacaatttccgctcactttcggcggagggaccaaggtggagatcaaacg

SEQ ID NO: 60 - protein, 112 aa, Homo sapiens
DIVMTQSPLSSPVTLGQPASISCRYSQSLVHRDGNTYLSWLQQRPGQPPRLVIHKISNRFSGVPDRFSGSGAGTDFTLKISRVEAEDVGFYYCMQTTQFPLTFGGGTKVEIK

SEQ ID NO: 61 - DNA, 353 bp, Homo sapiens
caggttcagctggtgcagtctggagctgaagtgaagaagcctggggcctcagtgaaggtctcctgcacggcttctggttacagtttttccagctatggtatcagctgggtgcgacaggcccctggacaagggcttgagtggatgggatggatcagcgcttacaatggtcacacacgctatgcacagaagctccagggcagagtcaccatgacctcagacacatccacgagcacagcctacatggagctgaggagcctgagatctgacgacacggccgtgtattactgtgcgagagccagcagctggtattttgactgctggggccagggaaccctggtcaccgtctcctcagc

SEQ ID NO: 62 - protein, 117 aa, Homo sapiens
QVQLVQSGAEVKKPGASVKVSCTASGYSFSSYGISWVRQAPGQGLEWMGWISAYNGHTRYAQKLQGRVTMTSDTSTSTAYMELRSLRSDDTAVYYCARASSWYFDCWGQGTLVTVSS

SEQ ID NO: 63 - DNA, 338 bp, Homo sapiens
gatattgtgatgacccagactccactctcctcacctgtcacccttggacagccggcctccatctcctgcaggtctagtcaaagcctcgtacacagtgatggaaacacctacttgagttggcttcagcagaggccaggccagcctccaagactcctaatttataagatttctaaccggttctctggggtcccagacagattcagtggcagtggggcagggacagatttcacactgaaaatcagcagggtggaagctgaggatgtcggggtttattactgcatgcaagctacacaatttccgctcactttcggcggagggaccaaggtggagatcaaacg

SEQ ID NO: 64 - protein, 112 aa, Homo sapiens
DIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQATQFPLTFGGGTKVEIK

SEQ ID NO: 65 - DNA, 353 bp, Homo sapiens
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcaacgcttacaatggtaacacaaactatgcacagaagctccggggcagagtcaccatgaccacagacacattcacgagcacagccgacatggagctgaggagcctgagatctgacgacacggccgtgtattactgtgcgagagcgtcgacagctatgggtgactactggggccagggaaccctggtcaccgtctcctcagc

SEQ ID NO: 66 - protein, 117 aa, Homo sapiens
QVQLVQSGAEVKKPGASVKVSCKASGYTFTSYGISWVRQAPGQGLEWMGWINAYNGNTNYAQKLRGRVTMTTDTFTSTADMELRSLRSDDTAVYYCARASTAMGDYWGQGTLVTVSS

SEQ ID NO: 67 - DNA, 338 bp, Homo sapiens
gagattgtgatgacccagactccactctcctcacctgtcacccttggacagccggcctccatctcctgcaagtctagtcaaagcctcgtacacagtgatggaaacacctacttgagttggcttcagcagaggccaggccagcctccaagactcctaatttataagatttctaaccggttctctggggtcccagacagattcagtggcagtggggcagggacagatttcacactgaaaatcagcagggtggaagctgaggatgtcggggtttattactgcatgcaaactacatactttccgctcactttcggcggagggaccaaggtagagatcaaacg

SEQ ID NO: 68 - protein, 112 aa, Homo sapiens
EIVMTQTPLSSPVTLGQPASISCKSSQSLVHSDGNTYLSWLQQRPGQPPRLLIYKISNRFSGVPDRFSGSGAGTDFTLKISRVEAEDVGVYYCMQTTYFPLTFGGGTKVEIK

SEQ ID NO: 69 - DNA, 371 bp, Homo sapiens
gaggtgcagctggtgcagtctggagcagaggtgaaaaagcccggggagtctctgaagatctcctgtaagggttctggatacagttttaccaactactggatcgcctgggtgcgccagatgcccgggaaaggcctggagtggatggggatcatctttcctggtgactctgataccagatacagcccggccttccaaggccaggtcaccatctcagccgacaagtccatcagcaccgcctacctacattggagcagcctgaaggcctcggacaccgccatgtattactgtgcgagacatagagattataccagtggcggaccggatgcttttgatgtctggggccaagggacaatggtcaccgtctcttcagc

SEQ ID NO: 70 - protein, 123 aa, Homo sapiens
EVQLVQSGAEVKKPGESLKISCKGSGYSFTNYWIAWVRQMPGKGLEWMGIIFPGDSDTRYSPAFQGQVTISADKSISTAYLHWSSLKASDTAMYYCARHRDYTSGGPDAFDVWGQGTMVTVSS

SEQ ID NO: 71 - DNA, 338 bp, Homo sapiens
gatattgtgatgacccagactccactctcctcacctgtcacccttggacagccggcctccatctcctgcaggtctagtcaaagcctcgtatacagtgatggaaacacctacttgagttggcttcaccagaggccaggccagcctccaagactcctaatttataagatttctaaccggttctctggggtcccagacagattcagtggcagtggggcagggacagatttcacactgaaaatcagcagggtggaggctgaggatgtcgggctttattactgcatgcaaactacacaatttcctctcactttcggcggagggaccaaggttaagatcaaacg

SEQ ID NO: 72 - protein, 112 aa, Homo sapiens
DIVMTQTPLSSPVTLGQPASISCRSSQSLVYSDGNTYLSWLHQRPGQPPRLLIYKISNRFSGVPDRFSGSGAGTDFTLKISRVEAEDVGLYYCMQTTQFPLTFGGGTKVKIK

SEQ ID NO: 73 - DNA, 365 bp, Homo sapiens
gaggtgcagctggtgcagtctggagcagaggtgaaaaagcccggggagtctctgaagatctcctgtaagggttctggatacagctttaccagctactggatcgcctgggtgcgccagatgcccgggaaaggcctggagtggatgggaatcatctatcctggtgactctgataccagatacagcccgtccttccaaggccaggtcaccatctcagccgacaagtccatcagcaccgcctacctgcagtggagcaacctgaaggcctcggacaccgccatgtattactgtgcgaggactgggagctactacaactactgcgggatggacgtctggggccaagggaccacggtcaccgtctcctcagc

SEQ ID NO: 74 - protein, 121 aa, Homo sapiens
EVQLVQSGAEVKKPGESLKISCKGSGYSFTSYWIAWVRQMPGKGLEWMGIIYPGDSDTRYSPSFQGQVTISADKSISTAYLQWSNLKASDTAMYYCARTGSYYNYCGMDVWGQGTTVTVSS

SEQ ID NO: 75 - DNA, 338 bp, Homo sapiens
gatattgtgatgacccagactccactctcctcacctgtcacccttggacagccggcctccatctcctgcaggtctagtcaaagcctcgtacacagtgatggaaacacctacttgagttggcttcagcagaggccaggccagcctccaagactcctaatttataagctttctaaccggttctctgggatcccagacagattcagtggcagtggggcagggacagatttcacactgaaaatcagcagggtggaagctgaggatgtcggggtttattactgcatgcaagctacacaatttcccctcactttcggcggagggaccaaggtggagatcaaacg

SEQ ID NO: 76 - protein, 112 aa, Homo sapiens
DIVMTQTPLSSPVTLGQPASISCRSSQSLVHSDGNTYLSWLQQRPGQPPRLLIYKLSNRFSGIPDRFSGSGAGTDFTLKISRVEAEDVGVYYCMQATQFPLTFGGGTKVEIK

SEQ ID NO: 77 - DNA, 365 bp, Homo sapiens
gaggtgcagctggtgcagtctggagcagaggtgaaaaagcccggggagtctctgaagatctcctgtaagggttctggatatagtttcaccaattactggatcgcctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtacgagacagggggattactatgatagtagtggccctgactactggggccagggaaccctggtcaccgtctcctcagc

SEQ ID NO: 78 - protein, 121 aa, Homo sapiens
EVQLVQSGAEVKKPGESLKISCKGSGYSFTNYWIAWVRQMPGKGLEWMGIIYPGDSDTRYSPSFQGQVTISADKSISTAYLQWSSLKASDTAMYYCTRQGDYYDSSGPDYWGQGTLVTVSS

SEQ ID NO: 79 - DNA, 338 bp, Homo sapiens
gatattgtgatgacccagactccactctcctcacctgtcacccttggacagccggcctccatttcctgcaggtctagtcaaagcctcgtatacagagatggaaacacctacttgagttggcttcagcagaggccaggccagcctccaagactcctaatttataagatttctaaccggttctctggggtcccagacagattcagtggcagtggggcagggacagatttcacactgaaaatcagcagggtggaagctgaggatgtcggggtttattactgcatgcaagctacacaatttcctctcactttcggcggagggaccaaggtggagatcaaacg

SEQ ID NO: 80 - protein, 112 aa, Homo sapiens
DIVMTQTPLSSPVTLGQPASISCRSSQSLVYRDGNTYLSWLQQRPGQPPRLLIYKISNRFSGVPDRFSGSGAGTDFTLKISRVEAEDVGVYYCMQATQFPLTFGGGTKVEIK

SEQ ID NO: 81 - DNA, 347 bp, Homo sapiens
caggtgcagctggtggagtctgcgggaggcgtggtccagcctgggaggtccctgagactctcctgtgcagcgtctggattcaccttcagtagttatggcatgcactgggtccgccaggctccaggcaaggggctggagtgggtggcagttatatggcatgatggaagtaaaaaatactatgaagactccgtgaagggccgattcaccatctccagagacaattccaagaacacgctgtatttgcaaatgaacagcctgagagccgaggacacggctgtgtattactgtgcgaggaactggttctttgactactggggccagggaaccctggtcaccgtctcctcagc

SEQ ID NO: 82 - protein, 115 aa, Homo sapiens
QVQLVESAGGVVQPGRSLRLSCAASGFTFSSYGMHWVRQAPGKGLEWVAVIWHDGSKKYYEDSVKGRFTISRDNSKNTLYLQMNSLRAEDTAVYYCARNWFFDYWGQGTLVTVSS

SEQ ID NO: 83 - DNA, 338 bp, Homo sapiens
aaaattgtgatgacccagactccactctcctcacctgtcacccttggacagccggcctccatctcctgcaggtctagtcaaagcctcgtacacagtgatggaaacacctacttgagttggtttcagcagaggccaggccagcctccaagactcctaattaataagatttctaaccggttctctggggtcccagacagattcagtggcagtggggcagggacagatttcacactgaaaatcagcagggtggaagctgaggatgtcggggtttattactgcatgcaagctacacaatttcctctcactttcggcggagggaccaaggtggagatcaaacg

SEQ ID NO: 84 - protein, 112 aa, Homo sapiens
KIVMTQTPLSSPVTLGQPASISCRSSQSLVHSDGNTYLSWFQQRPGQPPRLLINKISNRFSGVPDRFSGSGAGTDFTLKISRVEAEDVGVYYCMQATQFPLTFGGGTKVEIK

SEQ ID NO: 85 - DNA, 368 bp, Homo sapiens
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtactaccatatactacgcagactctgtgaagggccgattcaccatctccagggacaacgccaagaactcactgtatctgcaaatgaacagcctgagagccgaggacacggccgtgtattactgtgcgagagatctctactacggtggtaactcgtactactttgactactggggccagggaaccctggtcaccgtctcctcagc

SEQ ID NO: 86 - protein, 122 aa, Homo sapiens
QVQLVESGGGLVKPGGSLRLSCAASGFTFSDYYMSWIRQAPGKGLEWVSYISSSGTTIYYADSVKGRFTISRDNAKNSLYLQMNSLRAEDTAVYYCARDLYYGGNSYYFDYWGQGTLVTVSS

SEQ ID NO: 87 - DNA, 326 bp, Homo sapiens
tcttctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagcccgattctctggctccgactcaggaaacacagcttccttgaccatcactggggctcaggcggaagatgaggctgactattactgtaactcccgggacagcagtggtaaccatgtggtattcggcggagggaccaagctgaccgtcctagg

SEQ ID NO: 88 - protein, 108 aa, Homo sapiens
SSELTQDPAVSVALGQTVRITCQGDSLRSYYASWYQQKPGQAPVLVIYGKNNRPSGIPARFSGSDSGNTASLTITGAQAEDEADYYCNSRDSSGNHVVFGGGTKLTVL

SEQ ID NO: 89 - DNA, 365 bp, Homo sapiens
gaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagaaccgcctacctgcagtggagcagcctgaaggcctcggacaccgccatgtattactgtgcgagaattggggatcactaccattacaacggtatggacgtctggggccaagggaccacggtcaccgtctcctcagc

SEQ ID NO: 90 - protein, 121 aa, Homo sapiens
EVQLVQSGAEVKKPGESLKISCKGSGYSFTSYWIGWVRQMPGKGLEWMGIIYPGDSDTRYSPSFQGQVTISADKSIRTAYLQWSSLKASDTAMYYCARIGDHYHYNGMDVWGQGTTVTVSS

SEQ ID NO: 91 - DNA, 338 bp, Homo sapiens
gatattgtgatgacccagactccactctcctcacctgtcacccttggacagccggcctccatctcctgcaggtctagtcaaagcctcgtccacagtgatggaaacacctacttgagttggcttcagcagaggccaggccagcctccaagactcctactttataagaattctaaccggttctctggggtcccagacagattcagtggcagtggggcagggacagatttcacactgaaaatcagcagggtggaagctgaggatgtcggggtttattactgcatgcaagctacacaatttccgctcactttcggcggaggtaccaaggtggagatcaaacg

SEQ ID NO: 92 - protein, 112 aa, Homo sapiens
DIVMTQTPLSSPVTLGQPASISCRSSQSLVHSDGNTYLSWLQQRPGQPPRLLLYKNSNRFSGVPDRFSGSGAGTDFTLKISRVEAEDVGVYYCMQATQFPLTFGGGTKVEIK

SEQ ID NO: 93 - DNA, 365 bp, Homo sapiens
gaggtgcagctggtgcagtctggagcagaggtgaaaaagcccggggagtctctgaagatctcctgtaagggttctggatacagcttttccagctactggatcaactgggtgcgtcagatgcccgggaaaggcctggagtggatggggatcatctatcctggtgactctgataccagatacagcccgtccttccaaggccaggtcaccatctcagccgacaagtccatcagtaccgcctacctgcagtggcgcagcctgaaggcctcggacaccgccatttattattgtgcgagagtaggagattactactcctactacggtatggacgtctggggccaagggaccacggtcaccgtctcctcagc

SEQ ID NO: 94 - protein, 121 aa, Homo sapiens
EVQLVQSGAEVKKPGESLKISCKGSGYSFSSYWINWVRQMPGKGLEWMGIIYPGDSDTRYSPSFQGQVTISADKSISTAYLQWRSLKASDTAIYYCARVGDYYSYYGMDVWGQGTTVTVSS

SEQ ID NO: 95 - DNA, 338 bp, Homo sapiens
gatattgtgatgacccagactccaccctcctcacctgtcacccgtggacagccggcctccatctcctgcaggtctagtcaaagcctcgtacacagtgatggaaaaacctacttgagttggcttcagcagaggccaggccagcctccaagactcctaatttataagatttctaaccggttttctggggtcccagggagattcagtggcagtggggcagggacagatttcacactgaaaatcagcagggtggaagctgaggatgtcggggtttattactgcatgcaagctacacaatttcctctcactttcggcggagggaccaaggtggagatcaaacg

SEQ ID NO: 96 - protein, 112 aa, Homo sapiens
DIVMTQTPPSSPVTRGQPASISCRSSQSLVHSDGKTYLSWLQQRPGQPPRLLIYKISNRFSGVPGRFSGSGAGTDFTLKISRVEAEDVGVYYCMQATQFPLTFGGGTKVEIK

SEQ ID NO: 97 - DNA, 365 bp, Homo sapiens
gaggtgcagctggtgcagtctggagcagaggtgaaaaagcccggggagtctctgaagatctcctgtaagggttctggatacagctttaccaactactggatcaactgggtgcgccagatgcccgggaaaggcctggagtggatggggatcatctatcctggtgactctgataccagatacagcccgtccttccaaggccaggtcaccatctcagccgacaagtccatcaacaccgcctacctgcagtggagcagcctgaaggcctcggacaccgccatgtattactgtgcgagacagggaggacactactactactccggtatggacgtctggggtcaagggaccacggtcaccgtctcctcagc

SEQ ID NO: 98 - protein, 121 aa, Homo sapiens
EVQLVQSGAEVKKPGESLKISCKGSGYSFTNYWINWVRQMPGKGLEWMGIIYPGDSDTRYSPSFQGQVTISADKSINTAYLQWSSLKASDTAMYYCARQGGHYYYSGMDVWGQGTTVTVSS

SEQ ID NO: 99 - DNA, 338 bp, Homo sapiens
gatattgtgatgacccagactccactctcctcacctgtcacccttggacagccggcctccatctcctgcaggtcgcgtcaaagcctcctacacagtgatggaaacacctacttgagttggcttcagcagaggccaggccagcctccaagactcctaatttataagctttctaaccgggtctctggggtcccagacagattcagtggcagtggggcagggacagatttcacactgaaaatcagcagggtggaagctgaggatgtcggggtttattactgcatgcaatctacacaatttccgctcactttcggcggagggaccaaggtgaagatcaaacg

SEQ ID NO: 100 - protein, 112 aa, Homo sapiens
DIVMTQTPLSSPVTLGQPASISCRSRQSLLHSDGNTYLSWLQQRPGQPPRLLIYKLSNRVSGVPDRFSGSGAGTDFTLKISRVEAEDVGVYYCMQSTQFPLTFGGGTKVKIK

SEQ ID NO: 101 - DNA, 365 bp, Homo sapiens
gaggtgcagctggtgcagtctggagcagaggtgaaaaagcccggggagtctctgaagatctcctgtaagggttctagaaacagctttaccaactactggatcggctgggtgcgccagatgcccgggaaaggcctggagtggatggggatcatctatcctggtgactctgatacgagatacagcccgtccttccaaggccaggtcaccatctcagccgacaagtccatcagtaccgcctacctgcagtggagcagcctgaaggcctcggacaccgccatgtattactgtgcgcgaactgggagctactcctactactacggtatggacgtctggggccaagggaccacggtcaccgtctcctcagc

SEQ ID NO: 102 - protein, 121 aa, Homo sapiens
EVQLVQSGAEVKKPGESLKISCKGSRNSFTNYWIGWVRQMPGKGLEWMGIIYPGDSDTRYSPSFQGQVTISADKSISTAYLQWSSLKASDTAMYYCARTGSYSYYYGMDVWGQGTTVTVSS

SEQ ID NO: 103 - DNA, 338 bp, Homo sapiens
gatattgtgatgacccagactccactctcctcacctgtcacccttggacagccggcctccatctcctgcaggtctagtcaaagcctcgtacacagtgatagaaatacctacttgagttggcttcagcagaggccaggccagcctccaagactcctaatttataaggtttctaaccggttctctggggtcccagaaagattcagtggcagtgggacagggacagatttcacactgaaaatcagcagggtggaagctgaggatgtcggggtttattactgcgtgcaagaaacactatttcccatcaccatcggccaggggacacgactggagattaaacg

SEQ ID NO: 104 - protein, 112 aa, Homo sapiens
DIVMTQTPLSSPVTLGQPASISCRSSQSLVHSDRNTYLSWLQQRPGQPPRLLIYKVSNRFSGVPERFSGSGTGTDFTLKISRVEAEDVGVYYCVQETLFPITIGQGTRLEIK

SEQ ID NO: 105 - DNA, 365 bp, Homo sapiens
ggggtgcagctggttcagtctggagcagaggtgaaaaagcccggggagtctctgaagatctcctgtaagggttctggatacagttttaccagctactggatcggctgggtgcgccagatgcccgggaaaggcctggagtggatggggatcatctatcctggtgactctgataccagatacagcccgtccttccaaggccaggtcatcatttcagccgacaagtccatcagtaccgcctacctgcagtggagcagcctgaaggcctcggacaccgccatgtattactgtgcgagaactggggattactactcctaccacggaatggacgtctggggccaagggaccacggtcaccgtctcctcagc

SEQ ID NO: 106 - protein, 121 aa, Homo sapiens
GVQLVQSGAEVKKPGESLKISCKGSGYSFTSYWIGWVRQMPGKGLEWMGIIYPGDSDTRYSPSFQGQVIISADKSISTAYLQWSSLKASDTAMYYCARTGDYYSYHGMDVWGQGTTVTVSS

SEQ ID NO: 107 - DNA, 338 bp, Homo sapiens
gatattgtgatgacccagactccactctcctcacctgtcacccttggacagccggcctccatctcctgcaggtctagtcaaagcctcgtacacagtgatggaaacacctttttgagttggcttcagcagaggccaggccagcctccaagactcctaatttataagatttctaatcgcttctctggggtcccagacagattcagtggcagtggggcagggacagatttcacactgaaaatcagcagggtggaagctgaggatgtcgggatttattactgcatgcaagctacacaatttccgctcactttcggcggagggaccaaggtggagatcaaacg

SEQ ID NO: 108 - protein, 112 aa, Homo sapiens
DIVMTQTPLSSPVTLGQPASISCRSSQSLVHSDGNTFLSWLQQRPGQPPRLLIYKISNRFSGVPDRFSGSGAGTDFTLKISRVEAEDVGIYYCMQATQFPLTFGGGTKVEIK

SEQ ID NO: 109 - DNA, 374 bp, Homo sapiens
gaggtgcagctggtgcagtctggagcagaggtgaaaaagtccggggagtctctgaagatctcctgtaagggttctggatacagctttaccaacttctggatcggctgggtgcgccagatgcccgggaaaggcctggagtggatggggatcatctatcctggtgactctgataccagatacagcccgtccttccaaggccaggtcaccatctcagccgacaagtccatcagcaccacctacctgcagtggagcagcctgaaggcctcggacaccgccatgtattactgtgcgagacatcctccttatagtgggagctactacgctgatgcttttgatatctggggccaagggacaatggtcaccgtctcttcagc

SEQ ID NO: 110 - protein, 124 aa, Homo sapiens
EVQLVQSGAEVKKSGESLKISCKGSGYSFTNFWIGWVRQMPGKGLEWMGIIYPGDSDTRYSPSFQGQVTISADKSISTTYLQWSSLKASDTAMYYCARHPPYSGSYYADAFDIWGQGTMVTVSS

SEQ ID NO: 111 - DNA, 338 bp, Homo sapiens
gatattgtgctgacccagactccactctcctcacctgtcacccttggacagccggcctccatctcctgcaggtctagtcaaagcctcgtacacagtgatggacacacctacttgagttggcttcagcagaggccaggccagcctccaagactcctaatttataagatttctaaccggttctctggggtcccagacagattcagtggcagtggggcagggacagatttcacactgaaaatcagcagggtgggagctgaggatgtcggggtttattactgcatgcaagctacacaatttccgctcactttcggcggagggaccaaggtggagatcaaacg

SEQ ID NO: 112 - protein, 112 aa, Homo sapiens
DIVLTQTPLSSPVTLGQPASISCRSSQSLVHSDGHTYLSWLQQRPGQPPRLLIYKISNRFSGVPDRFSGSGAGTDFTLKISRVGAEDVGVYYCMQATQFPLTFGGGTKVEIK

SEQ ID NO: 113 - DNA, 374 bp, Homo sapiens
gaggtgcagctggtgcagtctggagcagaggtgaaaaagcccggggagtctctgaagatctcctgtaagggttctggatacagttttaccagcttctggatcggctgggtgcgccagatgcccgggaaaggcctggagtggatggggatcatctatcctggtgactctgataccagatacagcccgtccttccgaggcctggtcaccatctcagccgacaagtccatcagcaccgcctacctgcactggagcagcctgaaggcctcggacaccgccatcttttactgtgcgagacatcctccttatagtgggagctactacgctgatgcttttgatatctggggccaagggacaatggtcaccgtctcttcagc

SEQ ID NO: 114 - protein, 124 aa, Homo sapiens
EVQLVQSGAEVKKPGESLKISCKGSGYSFTSFWIGWVRQMPGKGLEWMGIIYPGDSDTRYSPSFRGLVTISADKSISTAYLHWSSLKASDTAIFYCARHPPYSGSYYADAFDIWGQGTMVTVSS

SEQ ID NO: 115 - DNA, 338 bp, Homo sapiens
gatattgtgatgacccagactccactctcctcacctgtcacccttggacagccggcctccatctcctgcaggtctagtcaaagcctcgtaagcagtgatggaaacacctacttgagttggcttcagcagaggccaggccagcctccaagactcctaatttataagatttctaacctattctctggggtcccagacagattcagtggcagtggggcagggacagatttcacactgaagatcagcagggtggaagctgaggatgtcgggctttattactgcatgcaagctacacaatttccgctcactttcggcggagggaccaaggtggagatcaaacg

SEQ ID NO: 116 - protein, 112 aa, Homo sapiens
DIVMTQTPLSSPVTLGQPASISCRSSQSLVSSDGNTYLSWLQQRPGQPPRLLIYKISNLFSGVPDRFSGSGAGTDFTLKISRVEAEDVGLYYCMQATQFPLTFGGGTKVEIK

SEQ ID NO: 117 - DNA, 365 bp, Homo sapiens
gaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagaactggggattaccacaactactacggtatggacgtctggggccaagggaccacggtcaccgtctcctcagc

SEQ ID NO: 118 - protein, 121 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARTGDYHNYYGMDVWGQGTTVTVSS

SEQ ID NO: 119 - DNA, 338 bp, Homo sapiens
gatattgtgatgacccagagtccactctcctcacctgtcacccttggacagccggcctccatctcctgcaggtctagtcaaagcctcgtacacagtgatggaaacaccttcttgagttggcttcagcagaggccaggccagcctccaagactcctaatttataagatttctaaccggttctctggggtcccagacagattcagtggcagtggggcagggacagatttcacactgaaaatcagcagggtggaagctgaggatgtcggggtttattactgcattcaagctacacaatttccgctcactttcggcggagggaccaaggtggagatcaaacg

SEQ ID NO: 120 - protein, 112 aa, Homo sapiens
DIVMTQSPLSSPVTLGQPASISCRSSQSLVHSDGNTFLSWLQQRPGQPPRLLIYKISNRFSGVPDRFSGSGAGTDFTLKISRVEAEDVGVYYCIQATQFPLTFGGGTKVEIK

SEQ ID NO: 121 - DNA, 365 bp, Homo sapiens
gaggtgcagctggtgcagtctggagcagaggtgaaaaagcccggggagtctctgaagatctcctgtaagggttctggatacagctttaccagttactggatcggctgggtgcgccagatgcccgggaaaggcctggagtggatggggatcatctatcctgctgactctgataccagatacagcccgtccttccaaggccaggtcaccatctcagccgacaagtccctcaacaccgcctacctgcagtggcgcagcctgaaggcctcggacaccgccatgtactactgtgcgagaattggtgacttctactactattccggtatggacgtctggggccaagggaccacggtcaccgtctcctcagc

SEQ ID NO: 122 - protein, 121 aa, Homo sapiens
EVQLVQSGAEVKKPGESLKISCKGSGYSFTSYWIGWVRQMPGKGLEWMGIIYPADSDTRYSPSFQGQVTISADKSLNTAYLQWRSLKASDTAMYYCARIGDFYYYSGMDVWGQGTTVTVSS

SEQ ID NO: 123 - DNA, 341 bp, Homo sapiens
gatgttgtgatgactcagtctccactctccctgcccgtcacccttggacagccggcctccatctcctgcaggtctcgtcaaagcctcgtatacagtgatggaagcacctacttgaattggtttcagcagaggccaggccaatctccaaggcgcctcatttataaggtttctaactgggactctggggtcccagacagattcagcgccagtgggtcaggcactgatttcacactgaaaatcagcagggtggaggctgaggatgatggggtttatcactgcatgcaaggtacacactggcctttgctcgctttcggcggagggaccaaggtggagatcaaacg

SEQ ID NO: 124 - protein, 113 aa, Homo sapiens
DVVMTQSPLSLPVTLGQPASISCRSRQSLVYSDGSTYLNWFQQRPGQSPRRLIYKVSNWDSGVPDRFSASGSGTDFTLKISRVEAEDDGVYHCMQGTHWPLLAFGGGTKVEIK

SEQ ID NO: 125 - DNA, 365 bp, Homo sapiens
gaggtgcagctggtgcagtctggagcagaggtgaaaaagcccggggagtctctgaagatctcctgtaagggttctggatacatctttaccagctactggatcgcctgggtgcgccagatgcccgggaaaggcctggagtggatggggatcatctatcctggtgactctgataccagatacagcccgtccttccaaggccagatcaccatctcagccgacaagtccatcagcaccgcctacctgcagtggagcagcctgaaggcctcggacaccgccatgtattactgtgcgagagtgggaactactaactactactacggtatggacgtctggggccaagggacctcggtcaccgtctcctcagc

SEQ ID NO: 126 - protein, 121 aa, Homo sapiens
EVQLVQSGAEVKKPGESLKISCKGSGYIFTSYWIAWVRQMPGKGLEWMGIIYPGDSDTRYSPSFQGQITISADKSISTAYLQWSSLKASDTAMYYCARVGTTNYYYGMDVWGQGTSVTVSS

SEQ ID NO: 127 - DNA, 338 bp, Homo sapiens
gatattgtgatgacccagactccactctcctcacctgtcacccttggacagccggcctccatctcctgcaggtctagtcaaagcctcgtacacagtgatggaaacaccttcttgagttggcttcagcagaggccaggccagcctccaagactcctaatttataagatttctaaccggttctctggggtcccagacagattcagtggcagtggggcagggacagatttcacactgaaaatcagcagggtggaagctgaggatgtcggggtttattactgcatgcaagctacgcagtttccgctcactttcggcggaggcaccaaggtggagatcaaacg

SEQ ID NO: 128 - protein, 112 aa, Homo sapiens
DIVMTQTPLSSPVTLGQPASISCRSSQSLVHSDGNTFLSWLQQRPGQPPRLLIYKISNRFSGVPDRFSGSGAGTDFTLKISRVEAEDVGVYYCMQATQFPLTFGGGTKVEIK

SEQ ID NO: 129 - DNA, 365 bp, Homo sapiens
gaggtgcagctggtgcagtctggagcagaggtgaaaaagcccggggagtctctgaagatctcctgtaagggttctggatacagctttaccacctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagaattggggattactactcctattccggtttggacgtctggggccaagggaccacggtcaccgtctcctcagc

SEQ ID NO: 130 - protein, 121 aa, Homo sapiens
EVQLVQSGAEVKKPGESLKISCKGSGYSFTTYWIGWVRQMPGKGLEWMGIIYPGDSDTRYSPSFQGQVTISADKSISTAYLQWSSLKASDTAMYYCARIGDYYSYSGLDVWGQGTTVTVSS

SEQ ID NO: 131 - DNA, 338 bp, Homo sapiens
gatattgtgatgacccagactccactctcctcacctgtcacccttggacagccggcctccatctcctgcaggtctagtcaaagcctcgtacacagtgatggacacacctacttgagttggcttcagcagaggccaggccagcctccaagactcctattttataagatttctaaccggttctctggggtcccagacagattcagtggcagtggggcagggacagatttcacactgaaaatcagcagggtggaagctgaggatgtcgggatttattactgcatgcaagctacacagtttccgctcactttcggcggagggaccaaggtggacatcaaacg

SEQ ID NO: 132 - protein, 112 aa, Homo sapiens
DIVMTQTPLSSPVTLGQPASISCRSSQSLVHSDGHTYLSWLQQRPGQPPRLLFYKISNRFSGVPDRFSGSGAGTDFTLKISRVEAEDVGIYYCMQATQFPLTFGGGTKVDIK

SEQ ID NO: 133 - DNA, 368 bp, Homo sapiens
caggtacagctgcagcagtcaggtccaggactgatgaagccctcgcagaccctctcactcacctgtgccatctccggggacagtgtctcaagcaacagtgtttcttggaactggatcaggcagtccccatcgagaggccttgagtggctgggaaggacatactacaggtttaagtggtttaatgattatgcagtatctgtgaaaagtcgaataaccatcaacccagacacatccaagaaccagttctccctgcgactgaactctgtgactcccgaggacacggctctgtattactgtgcaagaatagatatctggaacgacgtctttgactactggggccagggaaccctggtcaccgtctcctcagc

SEQ ID NO: 134 - protein, 122 aa, Homo sapiens
QVQLQQSGPGLMKPSQTLSLTCAISGDSVSSNSVSWNWIRQSPSRGLEWLGRTYYRFKWFNDYAVSVKSRITINPDTSKNQFSLRLNSVTPEDTALYYCARIDIWNDVFDYWGQGTLVTVSS

SEQ ID NO: 135 - DNA, 347 bp, Homo sapiens
cagcctgtgctgactcagccaacttccctctcagcatctcctggagcatcagccagactcacctgcaccttgcgcagtggcatcaatcttggtagctacaggatattctggtaccagcagaagccagagagccctccccggtatctcctgagctattattcagactcaagaaagcatcagggctctggagtccccagccgcttctctggatccaaagatgcttcgagcaatgcagggattttagtcatctctgggctccagtctgaggatgaggctgactattactgtattttttggcacagcagtgcttgggtattcggcggagggaccaagttgaccgtcctagg

SEQ ID NO: 136 - protein, 115 aa, Homo sapiens
QPVLTQPTSLSASPGASARLTCTLRSGINLGSYRIFWYQQKPESPPRYLLSYYSDSRKHQGSGVPSRFSGSKDASSNAGILVISGLQSEDEADYYCIFWHSSAWVFGGGTKLTVL

SEQ ID NO: 137 - DNA, 23 bp, Homo sapiens
tcaggagttttgagagcaaaatg

SEQ ID NO: 138 - DNA, 23 bp, Homo sapiens
aacagaagaaatcacttaaggag

SEQ ID NO: 139 - protein, 114 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARTAMDYWGQGTLVTVSS

SEQ ID NO: 140 - protein, 115 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARSSWFDYWGQGTLVTVSS

SEQ ID NO: 141 - protein, 117 aa, Homo sapiens
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARYGGNYFDYWGQGTLVTVSS

SEQ ID NO: 142 - protein, 111 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAFDYWGQGTLVTVSS

SEQ ID NO: 143 - protein, 118 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARYYYYYGMDVWGQGTTVTVSS

SEQ ID NO: 144 - protein, 119 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARYSGSYYAFDIWGQGTMVTVSS

SEQ ID NO: 145 - protein, 121 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARVGATNYYYGMDVWGQGTTVTVSS

SEQ ID NO: 146 - protein, 119 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARTGTYYYGMDVWGQGTTVTVSS

SEQ ID NO: 147 - protein, 119 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARYYGSGSAFDIWGQGTMVTVSS

SEQ ID NO: 148 - protein, 117 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARYGSGSFDYWGQGTLVTVSS

SEQ ID NO: 149 - protein, 116 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARYYDSSDYWGQGTLVTVSS

SEQ ID NO: 150 - protein, 119 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARDFWSGYYFDYWGQGTLVTVSS

SEQ ID NO: 151 - protein, 121 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARDFWSGYYTGMDVWGQGTTVTVSS

SEQ ID NO: 152 - protein, 117 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARDYSNAFDIWGQGTMVTVSS

SEQ ID NO: 153 - protein, 117 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARYSSGAFDVWGQGTMVTVSS

SEQ ID NO: 154 - protein, 117 aa, Homo sapiens
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARWNFDYWGQGTLVTVSS

SEQ ID NO: 155 - protein, 108 aa, Homo sapiens
DVVMTQSPLSLPVTLGQPASISCRSSQSLVYSDGNTYLNWFQQRPGQSPRRLIYKVSNWDSGVPDRFSGSGSGTDFTLKISRVEAEDVGVYYCMQGTHFGPGTKVDIK

SEQ ID NO: 156 - protein, 112 aa, Homo sapiens
DVVMTQSPLSLPVTLGQPASISCRSSQSLVYSDGNTYLNWFQQRPGQSPRRLIYKVSNWDSGVPDRFSGSGSGTDFTLKISRVEAEDVGVYYCMQGTHWPLTFGGGTKVEIK

SEQ ID NO: 157 - protein, 112 aa, Homo sapiens
DVVMTQSPLSLPVTLGQPASISCRSSQSLVYSDGNTYLNWFQQRPGQSPRRLIYKVSNWDSGVPDRFSGSGSGTDFTLKISRVEAEDVGVYYCMQGTHWPITFGQGTRLEIK

SEQ ID NO: 158 - protein, 110 aa, Homo sapiens
DIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQATQFTFGGGTKVEIK

SEQ ID NO: 159 - protein, 112 aa, Homo sapiens
DIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQATQFPITFGQGTRLEIK

SEQ ID NO: 160 - protein, 105 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWTFGGGTKVEIK

SEQ ID NO: 161 - protein, 107 aa, Homo sapiens
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVFGGGTKLTVL

SEQ ID NO: 162 - protein, 114 aa, Homo sapiens
QPVLTQPTSLSASPGASARLTCTLRSGINLGSYRIFWYQQKPESPPRYLLSYYSDSSKHQGSGVPSRFSGSKDASSNAGILVISGLQSEDEADYYCMIWHSSAVFGGGTKLTVL

SEQ ID NO: 163 - protein, 12 aa, Homo sapiens
KISHFLKMESLN

SEQ ID NO: 164 - protein, 12 aa, Homo sapiens
ISHFLKMESLNF

SEQ ID NO: 165 - protein, 12 aa, Homo sapiens
SHFLKMESLNFI

SEQ ID NO: 166 - protein, 12 aa, Homo sapiens
HFLKMESLNFIR

SEQ ID NO: 167 - protein, 12 aa, Homo sapiens
FLKMESLNFIRA

SEQ ID NO: 168 - protein, 12 aa, Homo sapiens
LKMESLNFIRAH

SEQ ID NO: 169 - protein, 12 aa, Homo sapiens
KMESLNFIRAHT

SEQ ID NO: 170 - protein, 12 aa, Homo sapiens
MESLNFIRAHTP

SEQ ID NO: 171 - protein, 12 aa, Homo sapiens
ESLNFIRAHTPY

SEQ ID NO: 172 - protein, 12 aa, Homo sapiens
SLNFIRAHTPYI

SEQ ID NO: 173 - protein, 12 aa, Homo sapiens
LNFIRAHTPYIN

SEQ ID NO: 174 - protein, 12 aa, Homo sapiens
NFIRAHTPYINI

SEQ ID NO: 175 - protein, 12 aa, Homo sapiens
FIRAHTPYINIY

SEQ ID NO: 176 - protein, 12 aa, Homo sapiens
IRAHTPYINIYN

SEQ ID NO: 177 - protein, 12 aa, Homo sapiens
RAHTPYINIYNC

SEQ ID NO: 178 - protein, 12 aa, Homo sapiens
AHTPYINIYNCE

SEQ ID NO: 179 - protein, 12 aa, Homo sapiens
HTPYINIYNCEP

SEQ ID NO: 180 - protein, 12 aa, Homo sapiens
TPYINIYNCEPA

SEQ ID NO: 181 - protein, 12 aa, Homo sapiens
PYINIYNCEPAN

SEQ ID NO: 182 - protein, 12 aa, Homo sapiens
YINIYNCEPANP

SEQ ID NO: 183 - protein, 12 aa, Homo sapiens
INIYNCEPANPS

SEQ ID NO: 184 - protein, 12 aa, Homo sapiens
NIYNCEPANPSE

SEQ ID NO: 185 - protein, 12 aa, Homo sapiens
IYNCEPANPSEK

SEQ ID NO: 186 - protein, 12 aa, Homo sapiens
YNCEPANPSEKN

SEQ ID NO: 187 - protein, 12 aa, Homo sapiens
NCEPANPSEKNS

SEQ ID NO: 188 - protein, 12 aa, Homo sapiens
CEPANPSEKNSP

SEQ ID NO: 189 - protein, 12 aa, Homo sapiens
EPANPSEKNSPS

SEQ ID NO: 190 - protein, 12 aa, Homo sapiens
PANPSEKNSPST

SEQ ID NO: 191 - protein, 12 aa, Homo sapiens
ANPSEKNSPSTQ

SEQ ID NO: 192 - protein, 12 aa, Homo sapiens
NPSEKNSPSTQY

SEQ ID NO: 193 - protein, 12 aa, Homo sapiens
PSEKNSPSTQYC

SEQ ID NO: 194 - protein, 12 aa, Homo sapiens
SEKNSPSTQYCY

SEQ ID NO: 195 - protein, 12 aa, Homo sapiens
EKNSPSTQYCYS

SEQ ID NO: 196 - protein, 9 aa, Homo sapiens
NPSEKNSPS

SEQ ID NO: 197 - protein, 10 aa, Homo sapiens
ESLNFIRAHT

SEQ ID NO: 198 - protein, 43 aa, Homo sapiens
KISHFLKMESLNFIRAHTPYINIYNCEPANPSEKNSPSTQYCY

SEQ ID NO: 199 - protein, 43 aa, Mus musculus
TLSHFLKMRRLELIQTSKPYVDIYDCEPSNSSEKNSPSTQYCN

SEQ ID NO: 200 - protein, 10 aa, Homo sapiens
PANPSEKNSP

SEQ ID NO: 201 - protein, 10 aa, Homo sapiens
GYSFTSYWIG

SEQ ID NO: 202 - protein, 7 aa, Homo sapiens
KISNRFS

(19) United States
(12) Patent Application Publication
(10) Pub. No.: US 2007/0014720 A1
(43) Pub. Date: Jan. 18, 2007

(54) Antibodies directed to CD20 and uses thereof

(76) Inventors: Gadi Gazit-Bornstein, Mountain View, CA (US); Larry L. Green, San Francisco, CA (US); Xiao-Dong Yang, Palo Alto, CA (US); Christophe Queva, Waltham, MA (US); David Charles Blakey, Macclesfield (GB)

Correspondence Address: KNOBBE MARTENS OLSON & BEAR LLP, 2040 MAIN STREET, FOURTEENTH FLOOR, IRVINE, CA, 92614, US

(21) Appl. No.: 11/440,728

(22) Filed: May 25, 2006

Related U.S. Application Data
(60) Provisional application No. 60/686,992, filed on Jun. 2, 2005.

(51) Int. Cl. A61K 51/00; A61K 39/395; C07H 21/04; C12P 21/06; C07K 16/28

(52) U.S. Cl. 424/1.11; 424/144.1; 435/69.1; 435/320.1; 435/325; 530/388.22; 536/23.53

(57) ABSTRACT

Antibodies directed to the antigen CD20 and uses of such antibodies are disclosed herein. In particular, fully human monoclonal antibodies directed to the antigen CD20. Nucleotide sequences encoding, and amino acid sequences comprising, heavy and light chain immunoglobulin molecules, particularly sequences corresponding to contiguous heavy and light chain sequences spanning the framework regions and/or complementarity determining regions (CDR's), specifically from FR1 through FR4 or CDR1 through CDR3 are disclosed. Hybridomas or other cell lines expressing such immunoglobulin molecules and monoclonal antibodies are also disclosed.

### CROSS-REFERENCE TO RELATED APPLICATIONS

This application claims priority under 35 U.S.C. § 119 to U.S. Provisional Application Ser. No. 60/686,992, filed Jun. 2, 2005, which is incorporated herein by reference.

### REFERENCE TO SEQUENCE LISTING

The present application is being filed along with a CD-ROM containing a a file entitled ABXAZ.003A.TXT created on May 25, 2006 which is 137,380 bytes in size, containing a Sequence Listing in electronic format. The information on this CD-ROM is incorporated herein by reference in its entirety.

### BACKGROUND OF THE INVENTION

1. Field of the Invention

The invention relates to monoclonal antibodies against the target antigen CD20 and uses of such antibodies. More specifically, the invention relates to fully human monoclonal antibodies directed to CD20 and uses of these antibodies. Aspects of the invention also relate to hybridomas or other cell lines expressing such antibodies. The described antibodies are useful as diagnostics and for the treatment of diseases associated with the activity and/or overexpression of CD20, and/or the presence and/or activity of CD20⁺ cells.

2. Description of the Related Art

CD20 is a 33,000 MW glyco-phosphoprotein that is 298 amino acids in length. The human CD20 gene is 1653 base pairs in length. The 5′UTR is 147 base pairs in length. The coding sequence is 893 base pairs while the 3′UTR is 613 base pairs in length.

CD20 is expressed at high density only on the surface of normal and neoplastic cells of the B lymphocyte lineage and is thought to function as a receptor during B cell activation. Stem cells and B-cell progenitors apparently lack the CD20 antigen. The predicted amino acid sequence of CD20 reveals a highly hydrophobic protein with 4 membrane-spanning domains, with the amino and carboxy termini of the protein located within the cytoplasm. A short hydrophilic region is located between residues 142 and 185 and may be exposed on the cell surface.

Three isoforms of human CD20 having weights of 33, 35 and 37 kDa result from differential phophorylation of a single protein. CD20 does not share any significant homology with other known proteins. There is a 73% homology between human and mouse sequences with the greatest similarity in the transmembrane regions.

CD20 is closely associated with other proteins, in particular the C-terminal src kinase-binding protein (Cbp), CD40, and major histocompatibility complex Class II proteins (MHC II). Antibody binding to CD20 has been found in some cases to induce rapid translocation of the molecule to lipid rafts.

Several companies currently sell therapeutic agents that target the CD20 protein. Rituxan® (Rituximab) (Genentech, South San Francisco, Calif.), Tositumomab® (GlaxoSmithKline, Brentford, Middlesex, United Kingdom), and HuMax-CD20 (Genmab, Copenhagen, Denmark) are monoclonal antibody therapeutics that target the CD20 protein.

### SUMMARY OF THE INVENTION

Embodiments of the invention relate to targeted binding agents that specifically bind to CD20 and inhibit the growth of cells that express CD20. Mechanisms by which this can be achieved can include, and are not limited to, either inducing apoptosis of cells expressing CD20, inducing antibody dependent cellular cytotoxicity (ADCC) in cells expressing CD20, or inducing complement dependent cytotoxicity (CDC) in cells expressing CD20, thereby eradicating CD20 positive B-cells including CD20⁺ lymphoma cells, CD20+ leukemia cells and normal B-cells.

In one embodiment of the invention, the targeted binding agent is a fully human antibody that binds to CD20 and induces apoptosis of cells expressing CD20. Yet another embodiment of the invention is a fully human monoclonal antibody that binds to, CD20 and induces antibody dependent cellular cytotoxicity (ADCC) in cells expressing CD20. Another embodiment of the invention is a fully human monoclonal antibody that binds to CD20 and induces complement dependent cytotoxicity (CDC) in cells expressing CD20.

In some embodiments, the antibody binds to CD20 and induces apoptosis of cells expressing CD20 with an EC₅₀ of about 0.5 μg/ml or less in a standard CellTiterGlo viability assay of Ramos cells. In some embodiments, the antibody, or antigen-binding portion thereof, has an EC₅₀ of no more than about 0.4, 0.3, 0.2, 0.1, 0.09, 0.08, 0.07, 0.06, 0.05, 0.04, 0.03, or 0.02 μg/ml for inducing apoptosis of B-cells in a standard CellTiterGlo viability assay of Ramos cells. In some embodiments, the antibody, or antigen-binding portion thereof, binds to CD20 and induces apoptosis of cells expressing CD20 with an EC₅₀ of about 0.2 μg/ml or less in a standard Alamar Blue viability assay of Ramos cells. In some embodiments, the antibody, or binding portion thereof, has an EC₅₀ of no more than about 0.1, 0.09, 0.08, 0.07, 0.06, 0.05, or 0.04 μg/ml in a standard Alamar Blue viability assay of Ramos cells.

Another embodiment of the invention is an antibody that competes for binding with any of the targeted binding agents or antibodies described herein.

In one embodiment, the antibody binds CD20 with a K_{D} of less than 12 nanomolar (nM). In another embodiment, the antibody binds with a K_{D} less than 10 nM, 9 nM, 8 Nm, 7 nM, 6 nM, 5 nM, 4 nM, 3 nM, 2 nM or 1 nM. In one embodiment, the antibody binds with a K_{D} of 500, 100, 30, 20, 10, or 5 pM. Affinity and/or avidity measurements can be measured by FMAT, FACS, KinExA® and/or BIACORE®, as described herein.

In one embodiment, the antibody comprises a heavy chain amino acid sequence having a complementarity determining region (CDR) with one of the sequences shown in Table 8. It is noted that those of ordinary skill in the art can readily accomplish CDR determinations. See for example, Kabat et al., Sequences of Proteins of Immunological Interest, Fifth Edition, NIH Publication 91-3242, Bethesda Md. (1991), vols. 1-3. One embodiment is a targeted binding agent that binds CD20 and comprises a heavy chain complementarity determining region 1 (CDR1) having an amino acid sequence of GYSFTSYWIG (SEQ ID NO. 201).

Yet another embodiment is an antibody that binds to CD20 and comprises a light chain amino acid sequence having a CDR comprising one of the sequences shown in Table 9. In certain embodiments the antibody is a fully human monoclonal antibody. Accordingly, one embodiment is a targeted binding agent that binds CD20 and comprises a light chain complementarity determining region 2 (CDR2) having an amino acid sequence of KISNRFS (SEQ ID NO. 202).

A further embodiment is an antibody that binds to CD20 and comprises a heavy chain amino acid sequence having one of the CDR sequences shown in Table 8 and a light chain amino acid sequence having one of the CDR sequences shown in Table 9. In certain embodiments the antibody is a fully human monoclonal antibody. In some embodiments, the invention provides an antibody that binds the same epitope as any of the antibodies disclosed herein.

One embodiment provides a monoclonal antibody, or antigen-binding portion thereof, wherein the antibody, or binding portion, comprises a heavy chain polypeptide having the sequence of SEQ ID NO.:2. In one embodiment, the antibody, or binding portion thereof, further comprises a light chain polypeptide having the sequence of SEQ ID NO.:4. Another embodiment is a monoclonal antibody, or antigen-binding portion thereof, wherein the antibody, or binding portion, comprises a heavy chain polypeptide having the sequence of SEQ ID NO.:30. In one embodiment, the antibody, or binding portion thereof, further comprises a light chain polypeptide having the sequence of SEQ ID NO.:32. Still another embodiment is a monoclonal antibody, or antigen-binding portion thereof, wherein the antibody, or binding portion, comprises a heavy chain polypeptide having the sequence of SEQ ID NO.:46. In one embodiment, the antibody, or binding portion thereof, further comprises a light chain polypeptide having the sequence of SEQ ID NO.:48.

Further embodiments of the invention include human monoclonal antibodies that specifically bind to CD20, wherein the antibodies comprise a heavy chain complementarity determining region 1 (CDR1) corresponding to canonical class 1. The antibodies provided herein can also include a heavy chain complementarity determining region 2 (CDR2) corresponding to canonical class 2, a light chain complementarity determining region 1 (CDR1) corresponding to canonical class 4, a light chain complementarity determining region 2 (CDR2) corresponding to canonical class 1, and a light chain complementarity determining region 3 (CDR3) corresponding to canonical class 1.

Other embodiments of the invention include human monoclonal antibodies that bind CD20 and comprise a heavy chain polypeptide derived from a VH5-51 germ line sequence. Some embodiments of the invention include human monoclonal antibodies that bind CD20 and comprise a V_{κ} light chain. Still other embodiments of the invention include a monoclonal antibody that comprises a V_{κ} light chain paired with a heavy chain encoded by, or derived from, a VH5-51 heavy chain gene. In some embodiments, the V_{κ} light chain polypeptide is encoded by, or derived from, an A23 light chain gene.

Yet another embodiment is a targeted binding agent that binds to amino acid residues 171-179 of the human CD20 extracellular domain. In other embodiments, the invention provides a targeted binding agent that binds an epitope comprising the peptide NPSEKNSPS (SEQ ID NO. 196). In still other embodiments, the invention provides targeted binding agent that does not require Alanine 170 for binding to the extracellular domain of CD20.

One embodiment of the invention comprises fully human monoclonal antibodies 1.1.2 (ATCC Accession Number PTA-7329), 2.1.2 (ATCC Accession Number PTA-7328), and 1.5.3 (ATCC Accession Number PTA-7330) which specifically bind to CD20, as discussed in more detail below.

One embodiment of the invention is an antibody that binds to the same epitopes as monoclonal antibodies 1.1.2 (ATCC Accession Number PTA-7329), 2.1.2 (ATCC Accession Number PTA-7328), and 1.5.3 (ATCC Accession Number PTA-7330).

Other embodiments the invention provide compositions, including an antibody or functional fragment thereof, and a pharmaceutically acceptable carrier.

Still further embodiments of the invention include methods of effectively treating an animal suffering from a neoplastic disease, including selecting an animal in need of treatment for a neoplastic disease, and administering to the animal a therapeutically effective dose of a fully human monoclonal antibody that specifically binds to CD20.

Treatable neoplastic diseases, include, for example, lymphomas, including B-cell lymphomas, such as non-Hodgkin's lymphoma (NHL), including precursor B cell lymphoblastic leukemia/lymphoma and mature B cell neoplasms, such as B cell Chronic Lymphocytic Leukemia (CLL), small lymphocytic lymphoma (SLL), B cell prolymphocytic leukemia, lymphoplasmacytic lymphoma, mantle cell lymphoma (MCL), follicular lymphoma (FL), including low-grade, intermediate grade and high-grade FL, cutaneous follicle center lymphoma, marginal zone B lymphoma (MALT type, nodal and splenic type), hairy cell leukemia, diffuse large B cell lymphoma, Burkitt's lymphoma, plasmacytoma, plasma cell myeloma, post-transplant lymphoproliferative disorder, Waldenström's macroglobulinemia, and anaplastic large cell lymphoma (ALCL). In addition, examples include relapsed or refractory B-NHL following Rituxan® therapy.

Further embodiments of the invention include methods of effectively treating an animal suffering from an immune system disease, including selecting an animal in need of treatment for an immune system disease, and administering to the animal a therapeutically effective dose of a fully human monoclonal antibody that specifically binds to CD20.

Treatable immune system diseases include, for example, but not limited to, Crohn's disease, Wegener's Granulomatosis, psoriasis, psoriatic arthritis, dermatitis, systemic scleroderma and sclerosis, inflammatory bowel disease (IBD), ulcerative colitis, respiratory distress syndrome, meningitis encephalitis, uveitis, glomerulonephritis, eczema, asthma, atherosclerosis, leukocyte adhesion deficiency, multiple sclerosis, Raynaud's syndrome, Sjögren's syndrome, juvenile onset diabetes, Reiter's disease, Behcet's disease, immune complex nephritis, IgA nephropathy, IgM polyneuropathies, immune-mediated thrombocytopenias, such as acute idiopathic thrombocytopenic purpura and chronic idiopathic thrombocytopenic purpura, hemolytic anemia, myasthenia gravis, lupus nephritis, systemic lupus erythematosus, rheumatoid arthritis (RA), atopic dermatitis, pemphigus, Grave's disease, Hashimoto's thyroiditis, Omenn's syndrome, chronic renal failure, acute infectious mononucleosis, HIV, and herpes virus associated diseases. Additional disorders include severe acute respiratory distress syndrome and choreoretinitis. Other examples are diseases and disorders caused by infection of B-cells with virus, such as Epstein Barr virus (EBV).

Additional embodiments of the invention include methods of inhibiting B-cell tumor growth in an animal. These methods include selecting an animal in need of treatment for B-cell tumor growth, and administering to the animal a therapeutically effective dose of a fully human monoclonal antibody wherein said antibody specifically binds to CD20.

Further embodiments of the invention include the use of an antibody in the preparation of medicament for the treatment of diseases involving CD20 expression in an animal, wherein the monoclonal antibody specifically binds to CD20.

In other embodiments, the antibodies described herein can be used for the preparation of a medicament for the treatment of neoplastic diseases in an animal, wherein the antibody specifically binds to CD20. Treatable neoplastic diseases include lymphomas, including B-cell lymphomas, such as non-Hodgkin's lymphoma (NHL).

Further embodiments include the use of an antibody in the preparation of a medicament for the treatment of immune diseases in an animal, wherein the antibody specifically binds to CD20.

Treatable diseases involving expression of CD20 include, for example, neoplastic diseases, such as NHL, including precursor B cell lymphoblastic leukemia/lymphoma and mature B cell neoplasms, such as B cell Chronic Lymphocytic Leukemia (CLL), small lymphocytic lymphoma (SLL), B cell prolymphocytic leukemia, lymphoplasmacytic lymphoma, mantle cell lymphoma (MCL), follicular lymphoma (FL), including low-grade, intermediate grade and high-grade FL, cutaneous follicle center lymphoma, marginal zone B lymphoma (MALT type, nodal and splenic type), hairy cell leukemia, diffuse large B cell lymphoma, Burkitt's lymphoma, plasmacytoma, plasma cell myeloma, post-transplant lymphoproliferative disorder, Waldenstrom's macroglobulinemia, and anaplastic large cell lymphoma (ALCL). In addition, examples include relapsed or refractory B-NHL following Rituxan® therapy. Examples of immune diseases include Crohn's disease, Wegener's Granulomatosis, psoriasis, psoriatic arthritis, dermatitis, systemic scleroderma and sclerosis, inflammatory bowel disease (IBD), ulcerative colitis, respiratory distress syndrome, meningitis encephalitis, uveitis, glomerulonephritis, eczema, asthma, atherosclerosis, leukocyte adhesion deficiency, multiple sclerosis, Raynaud's syndrome, Sjögren's syndrome, juvenile onset diabetes, Reiter's disease, Behcet's disease, immune complex nephritis, IgA nephropathy, IgM polyneuropathies, immune-mediated thrombocytopenias, such as acute idiopathic thrombocytopenic purpura and chronic idiopathic thrombocytopenic purpura, hemolytic anemia, myasthenia gravis, lupus nephritis, systemic lupus erythematosus, rheumatoid arthritis (RA), atopic dermatitis, pemphigus, Grave's disease, Hashimoto's thyroiditis, Omenn's syndrome, chronic renal failure, acute infectious mononucleosis, HIV, and herpes virus associated diseases. Additional disorders include severe acute respiratory distress syndrome and choreoretinitis. Other examples are diseases and disorders caused by infection of B-cells with virus, such as Epstein Barr virus (EBV).

Embodiments of the invention described herein relate to monoclonal antibodies that bind CD20 and affect CD20 function. Other embodiments relate to fully human anti-CD20 antibodies and anti-CD20 antibody preparations with desirable properties from a therapeutic perspective, including high binding affinity for CD20, the ability to eradicate CD20 positive B-cells and B-lymphoma cells in vitro and in vivo, the ability to induce apoptosis in vitro and in vivo, the ability to elicit ADCC activity in vitro and in vivo, the ability to induce CDC in vitro and in vivo, and/or the ability to inhibit B-cell tumor growth. Still other embodiments relate to fully human anti-CD20 antibodies and anti-CD20 antibody preparations that do not result in a significant Human Anti-Chimeric Antibody (HACA) response, thereby allowing for repeated administration.

Accordingly, one embodiment described herein includes isolated antibodies, or fragments of those antibodies, that bind to CD20. As known in the art, the antibodies can advantageously be, for example, polyclonal, oligoclonal, monoclonal, chimeric, humanized, and/or fully human antibodies. Embodiments of the invention described herein also provide cells for producing these antibodies.

It will be appreciated that embodiments of the invention are not limited to any particular form of an antibody or method of generation or production. For example, the anti-CD20 antibody can be a full-length antibody (e.g., having an intact human Fc region) or an antibody fragment (e.g., a Fab, Fab′ or F(ab′)₂). In addition, the antibody can be manufactured from a hybridoma that secretes the antibody, or from a recombinantly engineered cell that has been transformed or transfected with a gene or genes encoding the antibody. In addition, the antibodies can be single-domain antibodies such as camelid or human single VH or VL domains that bind to CD20.

Other embodiments of the invention include isolated nucleic acid molecules encoding any of the antibodies described herein, vectors having isolated nucleic acid molecules encoding anti-CD20 antibodies or a host cell transformed with any of such nucleic acid molecules. In addition, one embodiment of the invention is a method of producing an anti-CD20 antibody by culturing host cells under conditions wherein a nucleic acid molecule is expressed to produce the antibody followed by recovering the antibody.

A further embodiment herein includes a method of producing high affinity antibodies to CD20 by immunizing a mammal with cells expressing human CD20, isolated cell membranes containing human CD20, purified human CD20, or a fragment thereof, and/or one or more orthologous sequences or fragments thereof.

Other embodiments are based upon the generation and identification of isolated antibodies that bind specifically to CD20. CD20 is expressed on over 90% of B-cell lymphomas. Antibodies that mediate killing of B cells expressing CD20 can prevent CD20 induced tumor growth and other desired effects. Antibodies that mediate killing of non-malignant B cells can be used to treat or prevent immune diseases.

Another embodiment of the invention includes a method of diagnosing diseases or conditions in which an antibody prepared as described herein is utilized to detect the level of CD20 in a patient. In further embodiments, methods for the identification of risk factors, diagnosis of disease, and staging of disease is presented which involves the identification of the expression and/or overexpression of CD20 using anti-CD20 antibodies. In some embodiments, the methods comprise administering to a patient a fully human antibody conjugate that selectively binds to a CD20 protein on a cell. The antibody conjugate comprises an antibody that selectively binds to CD20 and a label. The methods further comprise observing the presence of the label in the patient. A relatively high amount of the label will indicate a relatively high risk of the disease and a relatively low amount of the label will indicate a relatively low risk of the disease. In one embodiment, the label is a green fluorescent protein.

The invention further provides methods for assaying the level of CD20 in a patient sample, comprising contacting an anti-CD20 antibody with a biological sample from a patient, and detecting the level of binding between said antibody and CD20 in said sample. In more specific embodiments, the biological sample is blood or serum.

Another embodiment of the invention includes a method for diagnosing a condition associated with the expression of CD20 in a cell by contacting serum or a cell with an anti-CD20 antibody, and thereafter detecting the presence of CD20.

In another embodiment, the invention includes an assay kit for detecting CD20 in mammalian tissues, cells, or body fluids to screen for diseases involving cells that express CD20. The kit includes an antibody that binds to CD20 and a means for indicating the reaction of the antibody with CD20, if present. Preferably the antibody is a monoclonal antibody. In one embodiment, the antibody that binds CD20 is labeled. In another embodiment the antibody is an unlabeled primary antibody and the kit further includes a means for detecting the primary antibody. In one embodiment, the means includes a labeled second antibody that is an anti-immunoglobulin. Preferably the antibody is labeled with a marker selected from the group consisting of a fluorochrome, an enzyme, a radionuclide and a radiopaque material.

Yet another embodiment includes methods for treating diseases or conditions associated with the expression of CD20 in a patient, by administering to the patient an effective amount of an anti-CD20 antibody. The anti-CD20 antibody can be administered alone, or can be administered in combination with additional antibodies or chemotherapeutic drug or radiation therapy. For example, a monoclonal, oligoclonal or polyclonal mixture of CD20 antibodies that induce apoptosis of B-lymphoma cells, and/or elicit ADCC, and/or induce CDC can be administered in combination with a drug shown to inhibit tumor cell proliferation directly. The method can be performed in vivo and the patient is preferably a human patient.

In some embodiments, the anti-CD20 antibodies can be modified to enhance their capability of fixing complement and participating in complement-dependent cytotoxicity (CDC). In other embodiments, the anti-CD20 antibodies can be modified to enhance their capability of activating effector cells and participating in antibody-dependent cytotoxicity (ADCC). In yet other embodiments, the anti-CD20 antibodies can be modified both to enhance their capability of activating effector cells and participating in antibody-dependent cytotoxicity (ADCC) and to enhance their capability of fixing complement and participating in complement-dependent cytotoxicity (CDC).

In another embodiment, the invention provides an article of manufacture including a container. The container includes a composition containing an anti-CD20 antibody, and a package insert or label indicating that the composition can be used to treat diseases characterized by the expression or overexpression of CD20.

In other embodiments, the invention provides a kit for treating diseases involving the expression of CD20, comprising anti-CD20 monoclonal antibodies and instructions to administer the monoclonal antibodies to a subject in need of treatment.

In another aspect, a method of selectively killing a cancerous cell in a patient is provided. The method comprises administering a fully human antibody conjugate to a patient. The fully human antibody conjugate comprises an antibody that can bind to the extracellular domain of CD20 and an agent. The agent is either a toxin, a radioisotope, or another substance that will kill a cancer cell. The antibody conjugate thereby selectively kills the cancer cell. The agent can be saporin.

In one aspect, a conjugated fully human antibody that binds to CD20 is provided. Attached to the antibody is an agent, and the binding of the antibody to a cell results in the delivery of the agent to the cell. In one embodiment, the above conjugated fully human antibody binds to an extracellular domain of CD20. In another embodiment, the antibody and conjugated toxin are internalized by a cell that expresses CD20. In another embodiment, the agent is a cytotoxic agent. In another embodiment, the agent is saporin. In still another embodiment, the agent is a radioisotope.

In some embodiments of the invention, the glycosylation patterns of the antibodies provided herein are modified to enhance ADCC and CDC effector function. See Shields R L et al., (2002) JBC. 277:26733; Shinkawa T et al., (2003) JBC. 278:3466 and Okazaki A et al., (2004) J. Mol. Biol., 336: 1239.

### BRIEF DESCRIPTION OF THE DRAWINGS

FIGS. 1 and 2 are graphs showing the results of CellTiterGlo cell viability assays without cross-linking of Ramos cells incubated with mAbs 1.1.2, 1.2.1, 1.3.3, 1.4.3, 1.5.3, 1.6.2 (FIG. 1), 1.9.2, 1.12.3, 1.13.2, 2.1.2, 2.2.2, 2.4.1 (FIG. 2), and the Rituxan® (“Rituximab”) antibody control. Percent viability of Ramos cells is shown on the y-axis and antibody concentration is shown on the x-axis.

FIGS. 3A-3D are graphs showing the results of Alamar Blue cell viability assays without crosslinker of Ramos cells incubated with mAbs 1.6.2, 1.5.3, 1.4.3, (FIG. 3A) 1.3.3, 1.1.2, B1 (FIG. 3B), 2.4.1, 2.2.2, 2.1.2, (FIG. 3C), 1.13.2, 1.12.3, B1 (FIG. 3D) Rituximab, IgM and IgG1. % viability is shown on the y-axis and antibody concentration is shown on the x-axis.

FIGS. 4A-4D are graphs showing the results of WST-1 cell viability assays without cross-linking of Ramos cells incubated with mAbs 1.1.2, 1.3.3, 1.4.3 (FIG. 4A), 1.5.3, 1.6.2 (FIG. 4B), 1.12.3, 1.13.2 (FIG. 4C), 2.1.2, 2.2.2, 2.4.1 (FIG. 4D), B1, Rituximab, IgG1, and IgM. % viability is shown on the y-axis and antibody concentration is shown on the x-axis.

FIGS. 5 and 6 are graphs showing the results of Annexin V/PI apoptosis assays without cross-linker of Ramos cells incubated with mAbs 1.1.2, 1.3.3, 1.4.3, 1.5.3, 1.6.2 (FIG. 5), 1.12.3, 1.13.2, 2.1.2, 2.2.2, 2.4.1 (FIG. 6), Rituximab and B1. % viability is shown on the y-axis and antibody concentration is on the x-axis.

FIGS. 7A-7D, 8A-8D, and 9A-9D are graphs showing the results of CDC assays of Ramos (FIGS. 7A-7D), Raji (FIGS. 8A-8D), and Daudi (FIGS. 9A-9D) cell lines, respectively, incubated with mAbs 1.1.2, 1.2.1, 1.3.3 (A), 1.4.3, 1.5.3, 1.6.2 (B), 1.9.2, 1.12.3, 1.13.2 (C), 2.1.2, 2.2.2, 2.4.1 (D), Rituximab, and IgG1 control. Percent viability is shown on the y-axis and antibody concentration is shown on the x-axis.

FIG. 10 is a graph showing results of an ADCC assay of Ramos cell line incubated with mAbs 2.1.2, 1.1.2, 1.5.3, 1.10.3.1, and 1.11.3.1, Rituximab, and IgG1 control. % viability is shown on the y-axis and antibody concentration is shown on the x-axis.

FIGS. 11-12 are graphs showing results of ADCC assays of Raji (FIG. 11), and Daudi (FIG. 12) cell lines incubated with mAbs 2.1.2, 1.1.2, 1.3.3, 1.5.3, Rituximab, and IgG1. % viability is shown on the y-axis and antibody concentration is shown on the x-axis.

FIG. 13 is a bar graph showing the results of whole blood assays of Raji, Ramos, and Daudi cells incubated with mAbs 1.1.2, 2.1.2, Rituximab, and IgG1 control using Whole Blood assays. Percent lysis is shown on the y-axis and Raji, Ramos, and Daudi cell lines, respectively, are shown on the x-axis. The results demonstrate mAbs 1.1.2 and 2.1.2 mediate greater cell lysis as compared to Rituximab.

FIGS. 14A and 14B are graphs showing results of whole blood assays of Karpas-422 (FIG. 14A) and EHEB (FIG. 14B) cell lines incubated with mAbs 2.1.2, 1.1.2, 1.5.3, 1.10.3.1, 1.11.3.1, Rituximab, and IgG1 control. % lysis is shown on the y-axis and antibody concentration is shown on the x-axis. The results demonstrate that EHEB and Karpas-422 cell lines are resistant to Rituximab treatment while the above anti-CD20 antibodies mediated significantly higher levels of cell lysis.

FIG. 15 is a scatterplot showing the results of a whole blood assay comparison of lytic activity using mAbs 1.5.3, 1.1.2, and Rituximab in a panel of cell lines. Each symbol represents a human donor of whole blood. Percent lysis at 10 μg/ml is shown on the y-axis and the ARH-77, Daudi, EHEB, JMV2, MV3, Karpas422, Namalwa, Raji, Ramos, SC1, SU-DHL-4, and WSU-NHL cell lines, respectively, are shown on the x-axis.

FIG. 16 is a scatter plot showing the ratio of lysis in a whole blood assay in a panel of cell lines between mAb 1.1.2 and Rituximab and between mAb 1.5.3 and Rituximab. Each symbol represents a human donor of whole blood. The ratio between the percent lysis at 10 μg/ml for each anti-CD20 mAb and the percent lysis achieved by Rituximab at 10 μg/ml for the same blood donor is shown. The ARH-77, Daudi, EHEB, JMV2, JMV3, Karpas422, Namalwa, Raji, Ramos, SC1, SU-DHL-4 and WSU-NHL cell lines, respectively, are shown on the x-axis.

FIG. 17 is a bar graph showing lysis of Rituximab-resistant cells RR1-Raji in a whole blood assay by Rituximab, mAb 1.1.2, and mAb 1.5.3. The percentage lysis is shown on the y-axis and and Raji parental cells treated at a concentration of antibody of 1 μg/ml and 10 μg/ml, respectively and RR1-Raji cells treated at a concentration of antibody of 1 μg/ml and 10 μg/ml, respectively, are shown on the x-axis.

FIG. 18 is a bar graph showing lysis of Rituximab-resistant cells RR1-Ramos, RR6-Ramos, and RR8-Ramos in a whole blood assay by Rituximab and mAb 1.5.3. The percentage lysis is shown on the y-axis and Ramos parental cells treated at a concentration of antibody of 1 μg/ml and 10 μg/ml, respectively, RR1-Ramos cells treated at a concentration of antibody of 1 μg/ml and 10 μg/ml, respectively, RR6-Ramos cells treated at a concentration of antibody of 1 μg/ml and 10 μg/ml, respectively, RR8-Ramos cells treated at a concentration of antibody of 1 μg/ml and 10 μg/ml, respectively, are shown on the x-axis.

FIG. 19 is a line graph showing the effect of anti-CD20 antibodies, Rituximab, 2.1.2, 1.1.2, and 1.5.3 on mouse survival in a Ramos i.v. paralysis model (CB17 SCID). The results show that three anti-CD20 antibodies demonstrate potent anti-lymphoma activity when administered as a single dose monotherapy. The number of days of treatment post tumor cell implantation is shown on the x-axis and percent survival is shown on the y-axis.

FIG. 20 is a line graph showing the efficacy of anti-CD20 antibodies in the Daudi subcutaneous tumor model. The number of days of treatment after the time of tumor cell inoculation is shown on the x-axis and tumor volume in cubic millimeters is shown on the y-axis.

FIG. 21 is a line graph showing the efficacy of anti-CD20 antibodies in the Namalwa subcutaneous tumor model. The number of days of treatment after the time of tumor cell inoculation is shown on the x-axis and tumor volume in cubic millimeters is shown on the y-axis.

FIG. 22 is a line graph showing the efficacy of anti-CD20 antibodies in the RR1-Raji subcutaneous tumor model. The number of days of treatment after the time of tumor cell inoculation is shown on the x-axis and tumor volume in cubic millimeters is shown on the y-axis.

FIG. 23 is a line graph showing the efficacy of anti-CD20 antibodies in the RR6-Ramos subcutaneous tumor model. The number of days of treatment after the time of tumor cell inoculation is shown on the x-axis and tumor volume in cubic millimeters is shown on the y-axis.

FIG. 24 is a bar graph showing depletion of tissue B-cells in cynomolgus monkey following treatment with control vehicle (saline), Rituximab (10 mg/kg), and mAb 1.5.3 (10 mg/kg). The percentage tissue CD20+CD40+ is shown on the y-axis and auxiliary, mesenteric, and inguinal lymph node, bone marrow, and spleen samples are shown on the x-axis.

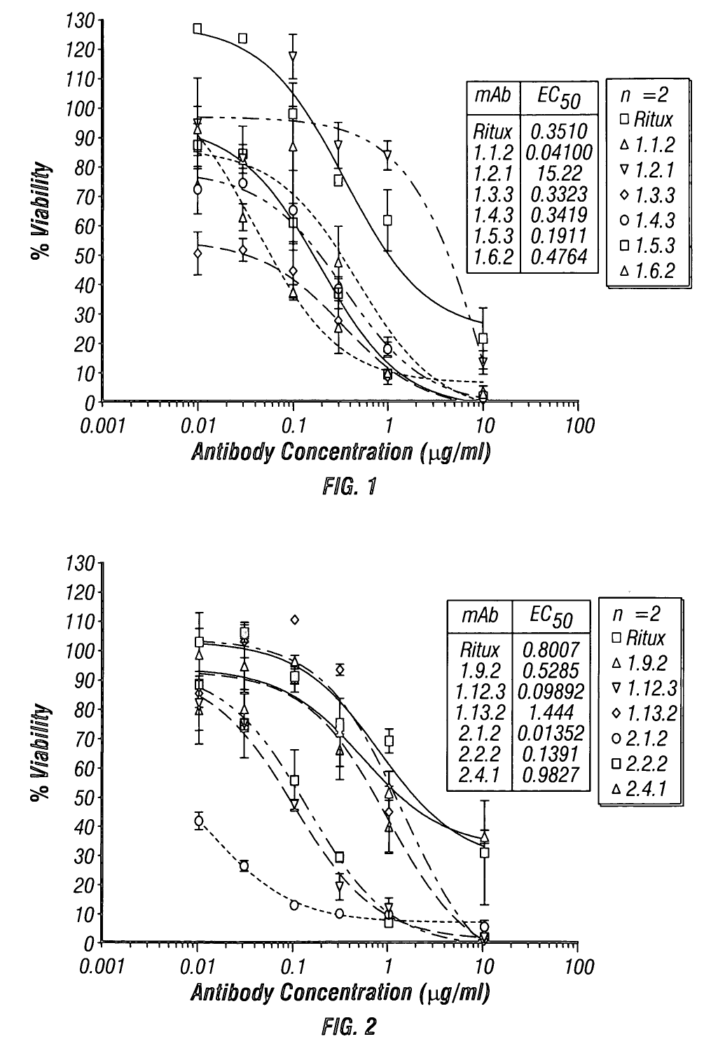

### DETAILED DESCRIPTION OF THE PREFERRED EMBODIMENT

Embodiments of the invention described herein relate to monoclonal antibodies that bind to CD20. In some embodiments, the antibodies bind to CD20 and induce apoptosis of B-lymphoma cells. Other embodiments of the invention include fully human anti-CD20 antibodies, and antibody preparations that are therapeutically useful. Such anti-CD20 antibody preparations preferably have desirable therapeutic properties, including strong binding affinity for CD20, the ability to induce apoptosis of B-lymphoma cells in vitro and in vivo, the ability to elicit ADCC activity in vitro and in vivo, and the ability to induce CDC activity in vitro and in vivo.

Embodiments of the invention also include isolated binding fragments of anti-CD20 antibodies. Preferably, the binding fragments are derived from fully human anti-CD20 antibodies. Exemplary fragments include Fv, Fab′ or other well known antibody fragments, as described in more detail below. Embodiments of the invention also include cells that express fully human antibodies against CD20. Examples of cells include hybridomas, or recombinantly created cells, such as Chinese hamster ovary (CHO) cells that produce antibodies against CD20.

In addition, embodiments of the invention include methods of using these antibodies for treating diseases. Anti-CD20 antibodies are useful for eradicating CD20 positive B cells and/or B-lymphoma cells. The mechanism of action can include inducing apoptosis of cells expressing CD20, inducing antibody dependent cellular cytotoxicity (ADCC) in cell expressing CD20, or inducing complement dependent cytotoxicity (CDC) in cells expressing CD20. Diseases that are treatable through this mechanism include, but are not limited to, neoplastic diseases, such as lymphomas, including B-cell lymphomas, such as Non Hodgkin's Lymphoma (NHL), including precursor B cell lymphoblastic leukemia/lymphoma and mature B cell neoplasms, such as B cell Chronic Lymphocytic Leukemia (CLL), small lymphocytic lymphoma (SLL), B cell prolymphocytic leukemia, lymphoplasmacytic lymphoma, mantle cell lymphoma (MCL), follicular lymphoma (FL), including low-grade, intermediate grade and high-grade FL, cutaneous follicle center lymphoma, marginal zone B lymphoma (Mucosa-Associated Lymphoid Tissue (MALT) type, nodal and splenic type), hairy cell leukemia, diffuse large B cell lymphoma, Burkitt's lymphoma, plasmacytoma, plasma cell myeloma, post-transplant lymphoproliferative disorder, Waldenström's macroglobulinemia, and anaplastic large cell lymphoma (ALCL). In addition, examples include relapsed or refractory B-NHL following Rituximab therapy. Immune diseases include Crohn's disease, Wegener's Granulomatosis, psoriasis, psoriatic arthritis, dermatitis, systemic scleroderma and sclerosis, inflammatory bowel disease (IBD), ulcerative colitis, respiratory distress syndrome, meningitis encephalitis, uveitis, glomerulonephritis, eczema, asthma, atherosclerosis, leukocyte adhesion deficiency, multiple sclerosis, Raynaud's syndrome, Sjögren's syndrome, juvenile onset diabetes, Reiter's disease, Behcet's disease, immune complex nephritis, IgA nephropathy, IgM polyneuropathies, immune-mediated thrombocytopenias, such as acute idiopathic thrombocytopenic purpura and chronic idiopathic thrombocytopenic purpura, hemolytic anemia, myasthenia gravis, lupus nephritis, systemic lupus erythematosus, rheumatoid arthritis (RA), atopic dermatitis, pemphigus, Grave's disease, Hashimoto's thyroiditis, Omenn's syndrome, chronic renal failure, acute infectious mononucleosis, Human Immunodeficiency Virus (HIV), and herpes virus associated diseases. Additional disorders include severe acute respiratory distress syndrome and choreoretinitis. Other examples are diseases and disorders caused by infection of B-cells with virus, such as Epstein Barr virus (EBV).

Other embodiments of the invention include diagnostic assays for specifically determining the presence and/or quantity of CD20 in a patient or biological sample. The assay kit can include anti-CD20 antibodies along with the necessary labels for detecting such antibodies. These diagnostic assays are useful to screen for CD20-related diseases including, but not limited to, neoplastic diseases, such as lymphomas, including B-cell lymphomas, such as Non Hodgkin's Lymphoma (NHL), including precursor B cell lymphoblastic leukemia/lymphoma and mature B cell neoplasms, such as B cell Chronic Lymphocytic Leukemia (CLL), small lymphocytic lymphoma (SLL), B cell prolymphocytic leukemia, lymphoplasmacytic lymphoma, mantle cell lymphoma (MCL), follicular lymphoma (FL), including low-grade, intermediate grade and high-grade FL, cutaneous follicle center lymphoma, marginal zone B lymphoma (MALT type, nodal and splenic type), hairy cell leukemia, diffuse large B cell lymphoma, Burkitt's lymphoma, plasmacytoma, plasma cell myeloma, post-transplant lymphoproliferative disorder, Waldenström's macroglobulinemia, and anaplastic large cell lymphoma (ALCL). In addition, examples include relapsed or refractory B-NHL following Rituximab therapy. Immune diseases include Crohn's disease, Wegener's Granulomatosis, psoriasis, psoriatic arthritis, dermatitis, systemic scleroderma and sclerosis, inflammatory bowel disease (IBD), ulcerative colitis, respiratory distress syndrome, meningitis encephalitis, uveitis, glomerulonephritis, eczema, asthma, atherosclerosis, leukocyte adhesion deficiency, multiple sclerosis, Raynaud's syndrome, Sjögren's syndrome, juvenile onset diabetes, Reiter's disease, Behcet's disease, immune complex nephritis, IgA nephropathy, IgM polyneuropathies, immune-mediated thrombocytopenias, such as acute idiopathic thrombocytopenic purpura and chronic idiopathic thrombocytopenic purpura, hemolytic anemia, myasthenia gravis, lupus nephritis, systemic lupus erythematosus, rheumatoid arthritis (RA), atopic dermatitis, pemphigus, Grave's disease, Hashimoto's thyroiditis, Omenn's syndrome, chronic renal failure, acute infectious mononucleosis, HIV, and herpes virus associated diseases. Additional disorders include severe acute respiratory distress syndrome and choreoretinitis. Other examples are diseases and disorders caused by infection of B-cells with virus, such as Epstein Barr virus (EBV).

In one embodiment there is provided a monoclonal antibody comprising a heavy chain polypeptide having the sequence of SEQ ID NO.:2. In one embodiment, the antibody further comprises a light chain polypeptide having the sequence of SEQ ID NO.:4. Another embodiment provides an antibody comprising a heavy chain polypeptide having the sequence of SEQ ID NO.:30. In one embodiment, the antibody further comprises a light chain polypeptide having the sequence of SEQ ID NO.:32. Still another embodiment provides an antibody comprising a heavy chain polypeptide having the sequence of SEQ ID NO.:46. In one embodiment, the antibody further comprises a light chain polypeptide having the sequence of SEQ ID NO.:48.

In one embodiment there is provided a hybridoma that produces the light chain and/or the heavy chain of antibody as described hereinabove. Preferably the hybridoma produces the light chain and/or the heavy chain of a fully human monoclonal antibody. More preferably the hybridoma produces the light chain and/or the heavy chain of the fully human monoclonal antibody 1.1.2 (ATCC Accession Number PTA-7329), 2.1.2 (ATCC Accession Number PTA-7328), and 1.5.3 (ATCC Accession Number PTA-7330). Alternatively the hybridoma produces an antibody that binds to the same epitope or epitopes as fully human monoclonal antibody 1.1.2 (ATCC Accession Number PTA-7329), 2.1.2 (ATCC Accession Number PTA-7328), and 1.5.3 (ATCC Accession Number PTA-7330).

In one embodiment there is provided a nucleic acid molecule encoding the light chain or the heavy chain of the antibody as described hereinabove.

Preferably there is provided a nucleic acid molecule encoding the light chain or the heavy chain of a fully human monoclonal antibody. More preferably there is provided a nucleic acid molecule encoding the light chain or the heavy chain of the fully human monoclonal antibody 1.1.2 (ATCC Accession Number PTA-7329), 2.1.2 (ATCC Accession Number PTA-7328), and 1.5.3 (ATCC Accession Number PTA-7330).

In one embodiment of the invention there is provided a vector comprising a nucleic acid molecule or molecules as described hereinabove, wherein the vector encodes a light chain and/or a heavy chain of an antibody as defined hereinabove.

In one embodiment of the invention there is provided a host cell comprising a vector as described hereinabove. Alternatively the host cell may comprise more than one vector.

In addition, one embodiment of the invention is a method of producing an antibody by culturing host cells under conditions wherein a nucleic acid molecule is expressed to produce the antibody, followed by recovery of the antibody.

In one embodiment of the invention there is provided a method of making an antibody comprising transfecting at least one host cell with at least one nucleic acid molecule encoding the antibody as described hereinabove, expressing the nucleic acid molecule in said host cell and isolating said antibody.

According to another aspect of the invention there is provided a method of inhibiting the growth of cells that express CD20 comprising administering a targeted binding agent as described hereinabove. The method may include selecting an animal in need of treatment for disease-related to CD20 expression, and administering to said animal a therapeutically effective dose of a targeted binding agent that specifically binds to CD20.

According to another aspect there is provided a method of treating an immune system disease in a mammal comprising administering a therapeutically effective amount of a targeted binding agent that specifically binds CD20. The method may include selecting an animal in need of treatment for an immune disease, and administering to the animal a therapeutically effective dose of a targeted binding agent that specifically binds CD20.

According to another aspect there is provided a method of treating a neoplastic disease in a mammal comprising administering a therapeutically effective amount of a targeted binding agent that specifically binds CD20. The method may include selecting an animal in need of treatment for a neoplastic disease, and administering to said animal a therapeutically effective dose of a targeted binding agent that specifically binds CD20. The agent can be administered alone, or can be administered in combination with a second anti-neoplastic agent selected from an antibody, a chemotherapeutic drug, or a radioactive drug.

According to another aspect there is provided a method of treating cancer in a mammal comprising administering a therapeutically effective amount of a targeted binding agent that specifically binds CD20. The method may include selecting an animal in need of treatment for cancer, and administering to said animal a therapeutically effective dose of a targeted binding agent that specifically binds CD20. The agent can be administered alone, or can be administered in combination with a second anti-neoplastic agent selected from an antibody, a chemotherapeutic drug, or a radioactive drug.

According to another aspect of the invention there is provided the use of a targeted binding agent that specifically binds CD20 for the manufacture of a medicament for the treatment of immune system diseases.

According to another aspect of the invention there is provided the use of a targeted binding agent that specifically binds CD20 for the manufacture of a medicament for the treatment of a neoplastic disease.

One embodiment the invention is particularly suitable for use in inhibiting B-cell tumor growth in patients with a tumor that is dependent alone, or in part, on CD20 expression.

Another embodiment of the invention includes an assay kit for detecting CD20 in mammalian tissues, cells, or body fluids to screen for neoplastic and/or immune system diseases. The kit includes a targeted binding agent that binds to CD20 and a means for indicating the reaction of the targeted binding agent with CD20, if present. The targeted binding agent may be a monoclonal antibody. In one embodiment, the antibody that binds CD20 is labeled. In another embodiment the antibody is an unlabeled primary antibody and the kit further includes a means for detecting the primary antibody. In one embodiment, the means includes a labeled second antibody that is an anti-immunoglobulin. Preferably the antibody is labeled with a marker selected from the group consisting of a fluorochrome, an enzyme, a radionuclide and a radio-opaque material.

Further embodiments, features, and the like regarding anti-CD20 antibodies are provided in additional detail below.

Definitions

Unless otherwise defined, scientific and technical terms used herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art.

Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (3rd ed., Cold Spring Harbor Laboratory Press, Cold Spring Harbor, N.Y. (2001)), which is incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

A compound refers to any small molecular weight compound with a molecular weight of less than about 2000 Daltons.

The term “CD20” refers to the 33,000 MW glyco-phosphoprotein CD20 that is 298 amino acids in length and encoded by the CD20 gene.

The term “isolated polynucleotide” as used herein shall mean a polynucleotide that has been isolated from its naturally occurring environment. Such polynucleotides may be genomic, cDNA, or synthetic. Isolated polynucleotides preferably are not associated with all or a portion of the polynucleotides they associate with in nature. The isolated polynucleotides may be operably linked to another polynucleotide that it is not linked to in nature. In addition, isolated polynucleotides preferably do not occur in nature as part of a larger sequence.

The term “isolated protein” referred to herein means a protein that has been isolated from its naturally occurring environment. Such proteins may be derived from genomic DNA, cDNA, recombinant DNA, recombinant RNA, or synthetic origin or some combination thereof, which by virtue of its origin, or source of derivation, the “isolated protein” (1) is not associated with proteins found in nature, (2) is free of other proteins from the same source, e.g. free of murine proteins, (3) is expressed by a cell from a different species, or (4) does not occur in nature.

The term “polypeptide” is used herein as a generic term to refer to native protein, fragments, or analogs of a polypeptide sequence. Hence, native protein, fragments, and analogs are species of the polypeptide genus. Preferred polypeptides in accordance with the invention comprise the human heavy chain immunoglobulin molecules and the human kappa light chain immunoglobulin molecules, as well as antibody molecules formed by combinations comprising the heavy chain immunoglobulin molecules with light chain immunoglobulin molecules, such as the kappa or lambda light chain immunoglobulin molecules, and vice versa, as well as fragments and analogs thereof. Preferred polypeptides in accordance with the invention may also comprise solely the human heavy chain immunoglobulin molecules or fragments thereof.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “operably linked” as used herein refers to positions of components so described that are in a relationship permitting them to function in their intended manner. For example, a control sequence “operably linked” to a coding sequence is connected in such a way that expression of the coding sequence is achieved under conditions compatible with the control sequences.

The term “control sequence” as used herein refers to polynucleotide sequences that are necessary either to effect or to affect the expression and processing of coding sequences to which they are connected. The nature of such control sequences differs depending upon the host organism; in prokaryotes, such control sequences generally include promoter, ribosomal binding site, and transcription termination sequence; in eukaryotes, generally, such control sequences may include promoters, enhancers, introns, transcription termination sequences, polyadenylation signal sequences, and 5′ and ′3 untranslated regions. The term “control sequences” is intended to include, at a minimum, all components whose presence is essential for expression and processing, and can also include additional components whose presence is advantageous, for example, leader sequences and fusion partner sequences.

The term “polynucleotide” as referred to herein means a polymeric form of nucleotides of at least 10 bases in length, either ribonucleotides or deoxynucleotides or a modified form of either type of nucleotide, or RNA-DNA hetero-duplexes. The term includes single and double stranded forms of DNA.

The term “oligonucleotide” referred to herein includes naturally occurring, and modified nucleotides linked together by naturally occurring, and non-naturally occurring linkages. Oligonucleotides are a polynucleotide subset generally comprising a length of 200 bases or fewer. Preferably, oligonucleotides are 10 to 60 bases in length and most preferably 12, 13, 14, 15, 16, 17, 18, 19, or 20 to 40 bases in length. Oligonucleotides are usually single stranded, e.g. for probes; although oligonucleotides may be double stranded, e.g. for use in the construction of a gene mutant. Oligonucleotides can be either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phosphoraniladate, phosphoroamidate, and the like. See e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990), the disclosures of which are hereby incorporated by reference. An oligonucleotide can include a label for detection, if desired.

The term “selectively hybridize” referred to herein means to detectably and specifically bind. Polynucleotides, oligonucleotides and fragments thereof selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, or antibody fragments and a nucleic acid sequence of interest will be at least 80%, and more typically with preferably increasing homologies of at least 85%, 90%, 95%, 99%, and 100%.

Two amino acid sequences are “homologous” if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least about 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program. It should be appreciated that there can be differing regions of homology within two orthologous sequences. For example, the functional sites of mouse and human orthologues may have a higher degree of homology than non-functional regions.

The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence.

In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or amino acid residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more preferably at least 99 percent sequence identity, as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2^{nd} Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Similarly, unless specified otherwise, the left-hand end of single-stranded polynucleotide sequences is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity. Preferably, residue positions that are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamic-aspartic, and asparagine-glutamine.

As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99% sequence identity to the antibodies or immunoglobulin molecules described herein. In particular, conservative amino acid replacements are contemplated. Conservative replacements are those that take place within a family of amino acids that have related side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are an aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding function or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the antibodies described herein.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991), which are each incorporated herein by reference.

The term “polypeptide fragment” as used herein refers to a polypeptide that has an amino-terminal and/or carboxy-terminal deletion, but where the remaining amino acid sequence is identical to the corresponding positions in the naturally-occurring sequence deduced, for example, from a full-length cDNA sequence. Fragments typically are at least 5, 6, 8 or 10 amino acids long, preferably at least 14 amino acids long, more preferably at least 20 amino acids long, usually at least 50 amino acids long, and even more preferably at least 70 amino acids long. The term “analog” as used herein refers to polypeptides which are comprised of a segment of at least 25 amino acids that has substantial identity to a portion of a deduced amino acid sequence and which has at least one of the following properties: (1) specific binding to a CD20, under suitable binding conditions, (2) ability to induce apoptosis of cells expressing CD20, (3) ability to elicit antibody dependent cellular cytotoxicity (ADCC), or (4) ability to induce complement dependent cytotoxicity (CDC). Typically, polypeptide analogs comprise a conservative amino acid substitution (or addition or deletion) with respect to the naturally-occurring sequence. Analogs typically are at least 20 amino acids long, preferably at least 50 amino acids long or longer, and can often be as long as a full-length naturally-occurring polypeptide.

Peptide analogs are commonly used in the pharmaceutical industry as non-peptide drugs with properties analogous to those of the template peptide. These types of non-peptide compound are termed “peptide mimetics” or “peptidomimetics”. Fauchere, J. Adv. Drug Res. 15:29 (1986); Veber and Freidinger TINS p. 392 (1985); and Evans et al. J. Med. Chem. 30:1229 (1987), which are incorporated herein by reference. Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from the group consisting of: —CH₂NH—, —CH₂S—, —CH₂—CH₂—, —CH═CH— (cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992), incorporated herein by reference); for example, by adding internal cysteine residues capable of forming intramolecular disulfide bridges which cyclize the peptide.

As used herein, the term “antibody” refers to a polypeptide or group of polypeptides that are comprised of at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site.

As used herein, a “targeted binding agent” is an antibody, or binding fragment thereof, that preferentially binds to a target site. In one embodiment, the targeted binding agent is specific for only one target site. In other embodiments, the targeted binding agent is specific for more than one target site. In one embodiment, the targeted binding agent may be a monoclonal antibody and the target site may be an epitope.

“Binding fragments” of an antibody are produced by recombinant DNA techniques, or by enzymatic or chemical cleavage of intact antibodies. Binding fragments include Fab, Fab′, F(ab′)₂, Fv, and single-chain antibodies. An antibody other than a “bispecific” or “bifunctional” antibody is understood to have each of its binding sites identical. An antibody substantially inhibits adhesion of a receptor to a counter-receptor when an excess of antibody reduces the quantity of receptor bound to counter-receptor by at least about 20%, 40%, 60% or 80%, and more usually greater than about 85% (as measured in an in vitro competitive binding assay).

An antibody may be oligoclonal, a polyclonal antibody, a monoclonal antibody, a chimeric antibody, a CDR-grafted antibody, a multi-specific antibody, a bispecific antibody, a catalytic antibody, a chimeric antibody, a humanized antibody, a fully human antibody, an anti-idiotypic antibody and antibodies that can be labeled in soluble or bound form as well as fragments, variants or derivatives thereof, either alone or in combination with other amino acid sequences provided by known techniques. An antibody may be from any species. The term antibody also includes binding fragments of the antibodies of the invention; exemplary fragments include Fv, Fab, Fab′, single stranded antibody (svFC), dimeric variable region (Diabody) and disulphide stabilized variable region (dsFv).

The term “epitope” includes any protein determinant capable of specific binding to an immunoglobulin or T-cell receptor. Epitopic determinants usually consist of chemically active surface groupings of molecules such as amino acids or sugar side chains and may, but not always, have specific three-dimensional structural characteristics, as well as specific charge characteristics. An antibody is said to specifically bind an antigen when the dissociation constant is ≦1 μM, preferably ≦100 nM and most preferably ≦10 nM.

The term “agent” is used herein to denote a chemical compound, a mixture of chemical compounds, a biological macromolecule, or an extract made from biological materials.

“Active” or “activity” in regard to a CD20 polypeptide refers to a portion of a CD20 polypeptide that has a biological or an immunological activity of a native CD20 polypeptide. “Biological” when used herein refers to a biological function that results from the activity of the native CD20 polypeptide. A preferred CD20 biological activity includes, for example, B-lymphocyte proliferation.

“Mammal” when used herein refers to any animal that is considered a mammal. Preferably, the mammal is human.

Digestion of antibodies with the enzyme, papain, results in two identical antigen-binding fragments, known also as “Fab” fragments, and a “Fc” fragment, having no antigen-binding activity but having the ability to crystallize. Digestion of antibodies with the enzyme, pepsin, results in the a F(ab′)₂ fragment in which the two arms of the antibody molecule remain linked and comprise two-antigen binding sites. The F(ab′)₂ fragment has the ability to crosslink antigen.

“Fv” when used herein refers to the minimum fragment of an antibody that retains both antigen-recognition and antigen-binding sites.

“Fab” when used herein refers to a fragment of an antibody that comprises the constant domain of the light chain and the CH1 domain of the heavy chain.

The term “mAb” refers to monoclonal antibody.

“Liposome” when used herein refers to a small vesicle that may be useful for delivery of drugs that may include the CD20polypeptide of the invention or antibodies to such a CD20 polypeptide to a mammal.

“Label” or “labeled” as used herein refers to the addition of a detectable moiety to a polypeptide, for example, a radiolabel, fluorescent label, enzymatic label chemiluminescent labeled or a biotinyl group. Radioisotopes or radionuclides may include ³H, ¹⁴C, ¹⁵N, ³⁵S, ⁹⁰Y, ⁹⁹Tc, ¹¹¹In, ¹²⁵I, ¹³¹I, fluorescent labels may include rhodamine, lanthanide phosphors or FITC and enzymatic labels may include horseradish peroxidase, βgalactosidase, luciferase, alkaline phosphatase.

The term “pharmaceutical agent or drug” as used herein refers to a chemical compound or composition capable of inducing a desired therapeutic effect when properly administered to a patient. Other chemistry terms herein are used according to conventional usage in the art, as exemplified by The McGraw-Hill Dictionary of Chemical Terms (Parker, S., Ed., McGraw-Hill, San Francisco (1985)), (incorporated herein by reference).

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

“Antibody-dependent cell-mediated cytotoxicity” and “ADCC” refer to a cell-mediated reaction in which non-specific cytotoxic cells that express Ig Fc receptors (FcRs) (e.g. Natural Killer (NK) cells, monocytes, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcRs expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362, or 5,821,337 can be performed. Useful effector cells for such assays include peripheral blood mononuclear cells (PBMC) and Natural Killer (NK) cells. Alternatively, or additionally, ADCC activity of the molecule of interest can be assessed in vivo, e.g., in an animal model such as that disclosed in Clynes et al. PNAS (USA) 95:652-656 (1988).

“Complement dependent cytotoxicity” and “CDC” refer to the mechanism by which antibodies carry out their cell-killing function. It is initiated by the binding of C1q, a constituent of the first component of complement, to the Fc domain of Igs, IgG or IgM, which are in complex with antigen (Hughs-Jones, N. C., and B. Gardner. 1979. Mol. Immunol. 16:697). C1q is a large, structurally complex glycoprotein of ˜410 kDa present in human serum at a concentration of 70 μg/ml (Cooper, N. R. 1985. Adv. Immunol. 37:151). Together with two serine proteases, C1r and C1s, C1q forms the complex C1, the first component of complement. At least two of the N-terminal globular heads of C1q must be bound to the Fc of Igs for C1 activation, hence for initiation of the complement cascade (Cooper, N. R. 1985. Adv. Immunol. 37:151).

“Whole blood assays” use unfractionated blood as a source of natural effectors. Blood contains complement in the plasma, together with FcR-expressing cellular effectors, such as polymorphonuclear cells (PMNs) and mononuclear cells (MNCs). Thus, whole blood assays allow simultaneous evaluation of the synergy of both ADCC and CDC effector mechanisms in vitro.

The term “patient” includes human and veterinary subjects.

Antibody Structure

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site.

Thus, an intact antibody has two binding sites. Except in bifunctional or bispecific antibodies, the two binding sites are the same.

The chains all exhibit the same general structure of relatively conserved framework regions (FR) joined by three hyper variable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR 1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann Clin. Exp. Immunol. 79: 315-321 (1990), Kostelny et al. J. Immunol. 148:1547-1553 (1992). Bispecific antibodies do not exist in the form of fragments having a single binding site (e.g., Fab, Fab′, and Fv).

Human Antibodies and Humanization of Antibodies

Human antibodies avoid some of the problems associated with antibodies that possess murine or rat variable and/or constant regions. The presence of such murine or rat derived proteins can lead to the rapid clearance of the antibodies or can lead to the generation of an immune response against the antibody by a patient. In order to avoid the utilization of murine or rat derived antibodies, fully human antibodies can be generated through the introduction of functional human antibody loci into a rodent, other mammal or animal so that the rodent, other mammal or animal produces fully human antibodies.

One method for generating fully human antibodies is through the use of XenoMouse® strains of mice that have been engineered to contain up to but less than 1000 kb-sized germline configured fragments of the human heavy chain locus and kappa light chain locus. The XenoMouse® strains are available from Abgenix, Inc. (Fremont, Calif.). Such mice, then, are capable of producing human immunoglobulin molecules and antibodies and are deficient in the production of murine immunoglobulin molecules and antibodies. Technologies utilized for achieving the same are disclosed in U.S. patent application Ser. No. 08/759,620, filed Dec. 3, 1996 and International Patent Application Nos. WO 98/24893, published Jun. 11, 1998 and WO 00/76310, published Dec. 21, 2000, the disclosures of which are hereby incorporated by reference. See also Mendez et al. Nature Genetics 15:146-156 (1997), the disclosure of which is hereby incorporated by reference.

In general, antibodies produced by the fused hybridomas were human IgG1 or IgG4 heavy chains with fully human kappa or lambda light chains. Antibodies can also be of other human isotypes, including IgG2 heavy chains. The antibodies possessed high affinities, typically possessing a Kd of from about 10⁻⁶ through about 10⁻¹² M or below, when measured against cells in FACS-based affinity measurement techniques. The affinity can also be measured by solid phase and solution phase techniques. In one embodiment, the antibodies described herein bind CD20 with a Kd of less than 12 nanomolar (nM) and induce apoptosis of B-lymphocytes. In some embodiments, the antibodies bind CD20 with a Kd of less than about 10, 9, 8, 7, 6, 5, or 4 nM.

As will be appreciated, anti-CD20 antibodies can be expressed in cell lines other than hybridoma cell lines. Sequences encoding particular antibodies can be used to transform a suitable mammalian host cell. Transformation can be by any known method for introducing polynucleotides into a host cell, including, for example packaging the polynucleotide in a virus (or into a viral vector) and transducing a host cell with the virus (or vector) or by transfection procedures known in the art, as exemplified by U.S. Pat. Nos. 4,399,216, 4,912,040, 4,740,461, and 4,959,455 (which patents are hereby incorporated herein by reference). The transformation procedure used depends upon the host to be transformed. Methods for introducing heterologous polynucleotides into mammalian cells are well known in the art and include dextran-mediated transfection, calcium phosphate precipitation, polybrene mediated transfection, protoplast fusion, electroporation, encapsulation of the polynucleotide(s) in liposomes, and direct microinjection of the DNA into nuclei.

Mammalian cell lines available as hosts for expression are well known in the art and include many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), human epithelial kidney 293 cells, and a number of other cell lines. Cell lines of particular preference are selected through determining which cell lines have high expression levels and produce antibodies with constitutive CD20 binding properties.

Anti-CD20 antibodies are useful in the detection of CD20 in patient samples and accordingly are useful as diagnostics for disease states as described herein. In addition, based on their ability to induce apoptosis, elicit ADCC, and/or induce CDC (as demonstrated in the Examples below), anti-CD20 antibodies have therapeutic effects in treating symptoms and conditions resulting from CD20 expression on B-cells. In specific embodiments, the antibodies and methods herein relate to the treatment of symptoms resulting from CD20 induced tumor growth. Further embodiments involve using the antibodies and methods described herein to treat neoplastic diseases, such as NHL, including precursor B cell lymphoblastic leukemia/lymphoma and mature B cell neoplasms, such as B cell Chronic Lymphocytic Leukemia (CLL), small lymphocytic lymphoma (SLL), B cell prolymphocytic leukemia, lymphoplasmacytic lymphoma, mantle cell lymphoma (MCL), follicular lymphoma (FL), including low-grade, intermediate grade and high-grade FL, cutaneous follicle center lymphoma, marginal zone B lymphoma (MALT type, nodal and splenic type), hairy cell leukemia, diffuse large B cell lymphoma, Burkitt's lymphoma, plasmacytoma, plasma cell myeloma, post-transplant lymphoproliferative disorder, Waldenström's macroglobulinemia, and anaplastic large cell lymphoma (ALCL). In addition, examples include relapsed or refractory B-NHL following Rituximab therapy. Immune diseases include Crohn's disease, Wegener's Granulomatosis, psoriasis, psoriatic arthritis, dermatitis, systemic scleroderma and sclerosis, inflammatory bowel disease (IBD), ulcerative colitis, respiratory distress syndrome, meningitis encephalitis, uveitis, glomerulonephritis, eczema, asthma, atherosclerosis, leukocyte adhesion deficiency, multiple sclerosis, Raynaud's syndrome, Sjögren's syndrome, juvenile onset diabetes, Reiter's disease, Behcet's disease, immune complex nephritis, IgA nephropathy, IgM polyneuropathies, immune-mediated thrombocytopenias, such as acute idiopathic thrombocytopenic purpura and chronic idiopathic thrombocytopenic purpura, hemolytic anemia, myasthenia gravis, lupus nephritis, systemic lupus erythematosus, rheumatoid arthritis (RA), atopic dermatitis, pemphigus, Grave's disease, Hashimoto's thyroiditis, Omenn's syndrome, chronic renal failure, acute infectious mononucleosis, HIV, and herpes virus associated diseases. Additional disorders include severe acute respiratory distress syndrome and choreoretinitis. Other examples are diseases and disorders caused by infection of B-cells with virus, such as Epstein Barr virus (EBV).

Antibody Sequences

Embodiments of the invention include the specific anti-CD20 antibodies listed below in Table 1. This table reports the identification number of each anti-CD20 antibody, along with the SEQ ID number of variable regions of the corresponding heavy chain and light chain genes.

Each antibody has been given an identification number that includes either two or three numbers separated by one or two decimal points. In some cases, only two identification numbers separated by one decimal point are listed. However, in some cases, several clones of one antibody were prepared. Although the clones have the identical nucleic acid and amino acid sequences as the parent sequence, they may also be listed separately, with the clone number indicated by the number to the right of a second decimal point. Thus, for example, the nucleic acid and amino acid sequences of antibody 1.2 are identical to the sequences of antibody 1.2.1, 1.2.2, and 1.2.3.

TABLE 1

| mAb ID | | SEQ ID |
|---|---|---|
| No.: | Sequence | NO: |
| 1.1.2 | Nucleotide sequence encoding the variable region of the heavy chain | 1 |
| | Amino acid sequence encoding the variable region of the heavy chain | 2 |
| | Nucleotide sequence encoding the variable region of the light chain | 3 |
| | Amino acid sequence encoding the variable region of the light chain | 4 |
| 1.10.3.1 | Nucleotide sequence encoding the variable region of the heavy chain | 5 |
| | Amino acid sequence encoding the variable region of the heavy chain | 6 |
| | Nucleotide sequence encoding the variable region of the light chain | 7 |
| | Amino acid sequence encoding the variable region of the light chain | 8 |
| 1.11.3.1 | Nucleotide sequence encoding the variable region of the heavy chain | 9 |
| | Amino acid sequence encoding the variable region of the heavy chain | 10 |
| | Nucleotide sequence encoding the variable region of the light chain | 11 |
| | Amino acid sequence encoding the variable region of the light chain | 12 |
| 1.12.1 | Nucleotide sequence encoding the variable region of the heavy chain | 13 |
| | Amino acid sequence encoding the variable region of the heavy chain | 14 |
| | Nucleotide sequence encoding the variable region of the light chain | 15 |
| | Amino acid sequence encoding the variable region of the light chain | 16 |
| 1.13.1 | Nucleotide sequence encoding the variable region of the heavy chain | 17 |
| | Amino acid sequence encoding the variable region of the heavy chain | 18 |
| | Nucleotide sequence encoding the variable region of the light chain | 19 |
| | Amino acid sequence encoding the variable region of the light chain | 20 |
| 1.2.1.1 | Nucleotide sequence encoding the variable region of the heavy chain | 21 |
| | Amino acid sequence encoding the variable region of the heavy chain | 22 |
| | Nucleotide sequence encoding the variable region of the light chain | 23 |
| | Amino acid sequence encoding the variable region of the light chain | 24 |
| 1.4.1 | Nucleotide sequence encoding the variable region of the heavy chain | 25 |
| | Amino acid sequence encoding the variable region of the heavy chain | 26 |
| | Nucleotide sequence encoding the variable region of the light chain | 27 |
| | Amino acid sequence encoding the variable region of the light chain | 28 |
| 1.5.3 | Nucleotide sequence encoding the variable region of the heavy chain | 29 |
| | Amino acid sequence encoding the variable region of the heavy chain | 30 |
| | Nucleotide sequence encoding the variable region of the light chain | 31 |
| | Amino acid sequence encoding the variable region of the light chain | 32 |
| 1.6.1 | Nucleotide sequence encoding the variable region of the heavy chain | 33 |
| | Amino acid sequence encoding the variable region of the heavy chain | 34 |
| | Nucleotide sequence encoding the variable region of the light chain | 35 |
| | Amino acid sequence encoding the variable region of the light chain | 36 |
| 1.7.1 | Nucleotide sequence encoding the variable region of the heavy chain | 37 |
| | Amino acid sequence encoding the variable region of the heavy chain | 38 |
| | Nucleotide sequence encoding the variable region of the light chain | 39 |
| | Amino acid sequence encoding the variable region of the light chain | 40 |
| 1.9.1 | Nucleotide sequence encoding the variable region of the heavy chain | 41 |
| | Amino acid sequence encoding the variable region of the heavy chain | 42 |
| | Nucleotide sequence encoding the variable region of the light chain | 43 |
| | Amino acid sequence encoding the variable region of the light chain | 44 |
| 2.1.1 | Nucleotide sequence encoding the variable region of the heavy chain | 45 |
| | Amino acid sequence encoding the variable region of the heavy chain | 46 |
| | Nucleotide sequence encoding the variable region of the light chain | 47 |
| | Amino acid sequence encoding the variable region of the light chain | 48 |
| 2.2.1 | Nucleotide sequence encoding the variable region of the heavy chain | 49 |
| | Amino acid sequence encoding the variable region of the heavy chain | 50 |
| | Nucleotide sequence encoding the variable region of the light chain | 51 |
| | Amino acid sequence encoding the variable region of the light chain | 52 |
| 2.4.1 | Nucleotide sequence encoding the variable region of the heavy chain | 53 |
| | Amino acid sequence encoding the variable region of the heavy chain | 54 |
| | Nucleotide sequence encoding the variable region of the light chain | 55 |
| | Amino acid sequence encoding the variable region of the light chain | 56 |
| 3.1.1 | Nucleotide sequence encoding the variable region of the heavy chain | 57 |
| | Amino acid sequence encoding the variable region of the heavy chain | 58 |
| | Nucleotide sequence encoding the variable region of the light chain | 59 |
| | Amino acid sequence encoding the variable region of the light chain | 60 |
| 3.2.1 | Nucleotide sequence encoding the variable region of the heavy chain | 61 |
| | Amino acid sequence encoding the variable region of the heavy chain | 62 |
| | Nucleotide sequence encoding the variable region of the light chain | 63 |
| | Amino acid sequence encoding the variable region of the light chain | 64 |
| 3.31 | Nucleotide sequence encoding the variable region of the heavy chain | 65 |
| | Amino acid sequence encoding the variable region of the heavy chain | 66 |
| | Nucleotide sequence encoding the variable region of the light chain | 67 |
| | Amino acid sequence encoding the variable region of the light chain | 68 |
| 3.4.1 | Nucleotide sequence encoding the variable region of the heavy chain | 69 |
| | Amino acid sequence encoding the variable region of the heavy chain | 70 |
| | Nucleotide sequence encoding the variable region of the light chain | 71 |
| | Amino acid sequence encoding the variable region of the light chain | 72 |
| 3.7.1 | Nucleotide sequence encoding the variable region of the heavy chain | 73 |
| | Amino acid sequence encoding the variable region of the heavy chain | 74 |
| | Nucleotide sequence encoding the variable region of the light chain | 75 |
| | Amino acid sequence encoding the variable region of the light chain | 76 |
| 4.2.1.1 | Nucleotide sequence encoding the variable region of the heavy chain | 77 |
| | Amino acid sequence encoding the variable region of the heavy chain | 78 |
| | Nucleotide sequence encoding the variable region of the light chain | 79 |
| | Amino acid sequence encoding the variable region of the light chain | 80 |
| 4.6.1 | Nucleotide sequence encoding the variable region of the heavy chain | 81 |
| | Amino acid sequence encoding the variable region of the heavy chain | 82 |
| | Nucleotide sequence encoding the variable region of the light chain | 83 |
| | Amino acid sequence encoding the variable region of the light chain | 84 |
| 6.3.1 | Nucleotide sequence encoding the variable region of the heavy chain | 85 |
| | Amino acid sequence encoding the variable region of the heavy chain | 86 |
| | Nucleotide sequence encoding the variable region of the light chain | 87 |
| | Amino acid sequence encoding the variable region of the light chain | 88 |
| 7.1.1 | Nucleotide sequence encoding the variable region of the heavy chain | 89 |
| | Amino acid sequence encoding the variable region of the heavy chain | 90 |
| | Nucleotide sequence encoding the variable region of the light chain | 91 |
| | Amino acid sequence encoding the variable region of the light chain | 92 |
| 7.17.1 | Nucleotide sequence encoding the variable region of the heavy chain | 93 |
| | Amino acid sequence encoding the variable region of the heavy chain | 94 |
| | Nucleotide sequence encoding the variable region of the light chain | 95 |
| | Amino acid sequence encoding the variable region of the light chain | 96 |
| 7.18.1 | Nucleotide sequence encoding the variable region of the heavy chain | 97 |
| | Amino acid sequence encoding the variable region of the heavy chain | 98 |
| | Nucleotide sequence encoding the variable region of the light chain | 99 |
| | Amino acid sequence encoding the variable region of the light chain | 100 |
| 7.21.1 | Nucleotide sequence encoding the variable region of the heavy chain | 101 |
| | Amino acid sequence encoding the variable region of the heavy chain | 102 |
| | Nucleotide sequence encoding the variable region of the light chain | 103 |
| | Amino acid sequence encoding the variable region of the light chain | 104 |
| 7.23.1 | Nucleotide sequence encoding the variable region of the heavy chain | 105 |
| | Amino acid sequence encoding the variable region of the heavy chain | 106 |
| 7.23.1.1 | Nucleotide sequence encoding the variable region of the light chain | 107 |
| k2 | Amino acid sequence encoding the variable region of the light chain | 108 |
| 7.24.1 | Nucleotide sequence encoding the variable region of the heavy chain | 109 |
| | Amino acid sequence encoding the variable region of the heavy chain | 110 |
| 7.24.1.1 | Nucleotide sequence encoding the variable region of the light chain | 111 |
| k2 | Amino acid sequence encoding the variable region of the light chain | 112 |
| 7.26.1 | Nucleotide sequence encoding the variable region of the heavy chain | 113 |
| | Amino acid sequence encoding the variable region of the heavy chain | 114 |
| | Nucleotide sequence encoding the variable region of the light chain | 115 |
| | Amino acid sequence encoding the variable region of the light chain | 116 |
| 7.28.1 | Nucleotide sequence encoding the variable region of the heavy chain | 117 |
| | Amino acid sequence encoding the variable region of the heavy chain | 118 |
| 7.28.1.1 | Nucleotide sequence encoding the variable region of the light chain | 119 |
| k2 | Amino acid sequence encoding the variable region of the light chain | 120 |
| 7.7.1 | Nucleotide sequence encoding the variable region of the heavy chain | 121 |
| | Amino acid sequence encoding the variable region of the heavy chain | 122 |
| | Nucleotide sequence encoding the variable region of the light chain | 123 |
| | Amino acid sequence encoding the variable region of the light chain | 124 |
| 7.8.1 | Nucleotide sequence encoding the variable region of the heavy chain | 125 |
| | Amino acid sequence encoding the variable region of the heavy chain | 126 |
| | Nucleotide sequence encoding the variable region of the light chain | 127 |
| | Amino acid sequence encoding the variable region of the light chain | 128 |
| 7.9.1 | Nucleotide sequence encoding the variable region of the heavy chain | 129 |
| | Amino acid sequence encoding the variable region of the heavy chain | 130 |
| | Nucleotide sequence encoding the variable region of the light chain | 131 |
| | Amino acid sequence encoding the variable region of the light chain | 132 |
| 8.2.1 | Nucleotide sequence encoding the variable region of the heavy chain | 133 |
| | Amino acid sequence encoding the variable region of the heavy chain | 134 |
| | Nucleotide sequence encoding the variable region of the light chain | 135 |
| | Amino acid sequence encoding the variable region of the light chain | 136 |

Therapeutic Administration and Formulations

Anti-CD20 antibodies can have therapeutic effects in treating symptoms and conditions related to CD20 expression. For example, the antibodies can induce apoptosis of cells expressing CD20, thereby inhibiting tumor growth, or the antibodies can be associated with an agent and deliver a lethal toxin to a targeted cell. In addition, the anti-CD20 antibodies are useful as diagnostics for the disease states, especially neoplastic and immune diseases.

If desired, the isotype of an anti-CD20 antibody can be switched, for example to take advantage of a biological property of a different isotype. For example, in some circumstances it can be desirable in connection with the generation of antibodies as therapeutic antibodies against CD20 that the antibodies be capable of fixing complement and participating in complement-dependent cytotoxicity (CDC). There are a number of isotypes of antibodies that are capable of the same, including, without limitation, the following: murine IgM, murine IgG2a, murine IgG2b, murine IgG3, human IgM, human IgA, human IgG1, and human IgG3. In other embodiments it can be desirable in connection with the generation of antibodies as therapeutic antibodies against CD20 that the antibodies be capable of binding Fc receptors on effector cells and participating in antibody-dependent cytotoxicity (ADCC). There are a number of isotypes of antibodies that are capable of the same, including, without limitation, the following: murine IgG2a, murine IgG2b, murine IgG3, human IgG1, and human IgG3. It will be appreciated that antibodies that are generated need not initially possess such an isotype but, rather, the antibody as generated can possess any isotype and the antibody can be isotype switched thereafter using conventional techniques that are well known in the art. Such techniques include the use of direct recombinant techniques (see e.g., U.S. Pat. No. 4,816,397), cell-cell fusion techniques (see e.g., U.S. Pat. Nos. 5,916,771 and 6,207,418), among others.

By way of example, the anti-CD20 antibodies discussed herein are fully human antibodies. If an antibody possessed desired binding to CD20, it could be readily isotype switched to generate a human IgM, human IgG1, or human IgG3 isotype, while still possessing the same variable region (which defines the antibody's specificity and some of its affinity). Such molecule would then be capable of fixing complement and participating in CDC and/or be capable of binding to Fc receptors on effector cells and participating in ADCC.

In the cell-cell fusion technique, a myeloma, CHO cell or other cell line is prepared that possesses a heavy chain with any desired isotype and another myeloma, CHO cell or other cell line is prepared that possesses the light chain. Such cells can, thereafter, be fused and a cell line expressing an intact antibody can be isolated.

Accordingly, as antibody candidates are generated that meet desired “structural” attributes as discussed above, they can generally be provided with at least certain of the desired “functional” attributes through isotype switching.

Embodiments of the invention include sterile pharmaceutical formulations of anti-CD20 antibodies that are useful as treatments for diseases. Such formulations would induce B-lymphoma cell apoptosis, thereby effectively treating pathological conditions where, for example, CD20 expression is abnormally elevated or CD20 expressing cells mediate disease states. Anti-CD20 antibodies preferably possess adequate affinity to specifically bind CD20, and preferably have an adequate duration of action to allow for infrequent dosing in humans. A prolonged duration of action will allow for less frequent and more convenient dosing schedules by alternate parenteral routes such as subcutaneous or intramuscular injection.

Sterile formulations can be created, for example, by filtration through sterile filtration membranes, prior to or following lyophilization and reconstitution of the antibody. The antibody ordinarily will be stored in lyophilized form or in solution. Therapeutic antibody compositions generally are placed into a container having a sterile access port, for example, an intravenous solution bag or vial having an adapter that allows retrieval of the formulation, such as a stopper pierceable by a hypodermic injection needle.

The route of antibody administration is in accord with known methods, e.g., injection or infusion by intravenous, intraperitoneal, intracerebral, intramuscular, intraocular, intraarterial, intrathecal, inhalation or intralesional routes, or by sustained release systems as noted below. The antibody is preferably administered continuously by infusion or by bolus injection.

An effective amount of antibody to be employed therapeutically will depend, for example, upon the therapeutic objectives, the route of administration, and the condition of the patient. Accordingly, it is preferred that the therapist titer the dosage and modify the route of administration as required to obtain the optimal therapeutic effect. Typically, the clinician will administer antibody until a dosage is reached that achieves the desired effect. The progress of this therapy is easily monitored by conventional assays or by the assays described herein.

Antibodies, as described herein, can be prepared in a mixture with a pharmaceutically acceptable carrier. This therapeutic composition can be administered intravenously or through the nose or lung, preferably as a liquid or powder aerosol (lyophilized). The composition can also be administered parenterally or subcutaneously as desired. When administered systemically, the therapeutic composition should be sterile, pyrogen-free and in a parenterally acceptable solution having due regard for pH, isotonicity, and stability. These conditions are known to those skilled in the art. Briefly, dosage formulations of the compounds described herein are prepared for storage or administration by mixing the compound having the desired degree of purity with physiologically acceptable carriers, excipients, or stabilizers. Such materials are non-toxic to the recipients at the dosages and concentrations employed, and include buffers such as TRIS HCl, phosphate, citrate, acetate and other organic acid salts; antioxidants such as ascorbic acid; low molecular weight (less than about ten residues) peptides such as polyarginine, proteins, such as serum albumin, gelatin, or immunoglobulins; hydrophilic polymers such as polyvinylpyrrolidinone; amino acids such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium and/or nonionic surfactants such as TWEEN, PLURONICS or polyethyleneglycol.

Sterile compositions for injection can be formulated according to conventional pharmaceutical practice as described in Remington: The Science and Practice of Pharmacy (20^{th} ed, Lippincott Williams & Wilkens Publishers (2003)). For example, dissolution or suspension of the active compound in a vehicle such as water or naturally occurring vegetable oil like sesame, peanut, or cottonseed oil or a synthetic fatty vehicle like ethyl oleate or the like can be desired. Buffers, preservatives, antioxidants and the like can be incorporated according to accepted pharmaceutical practice.

Suitable examples of sustained-release preparations include semipermeable matrices of solid hydrophobic polymers containing the polypeptide, which matrices are in the form of shaped articles, films or microcapsules. Examples of sustained-release matrices include polyesters, hydrogels (e.g., poly(2-hydroxyethyl-methacrylate) as described by Langer et al., J. Biomed Mater. Res., (1981) 15:167-277 and Langer, Chem. Tech., (1982) 12:98-105, or poly(vinylalcohol)), polylactides (U.S. Pat. No. 3,773,919, EP 58,481), copolymers of L-glutamic acid and gamma ethyl-L-glutamate (Sidman et al., Biopolymers, (1983) 22:547-556), non-degradable ethylene-vinyl acetate (Langer et al., supra), degradable lactic acid-glycolic acid copolymers such as the LUPRON Depot™ (injectable microspheres composed of lactic acid-glycolic acid copolymer and leuprolide acetate), and poly-D-(−)-3-hydroxybutyric acid (EP 133,988).

While polymers such as ethylene-vinyl acetate and lactic acid-glycolic acid enable release of molecules for over 100 days, certain hydrogels release proteins for shorter time periods. When encapsulated proteins remain in the body for a long time, they can denature or aggregate as a result of exposure to moisture at 37° C., resulting in a loss of biological activity and possible changes in immunogenicity. Rational strategies can be devised for protein stabilization depending on the mechanism involved. For example, if the aggregation mechanism is discovered to be intermolecular S—S bond formation through disulfide interchange, stabilization can be achieved by modifying sulfhydryl residues, lyophilizing from acidic solutions, controlling moisture content, using appropriate additives, and developing specific polymer matrix compositions.

Sustained-released compositions also include preparations of crystals of the antibody suspended in suitable formulations capable of maintaining crystals in suspension. These preparations when injected subcutaneously or intraperitonealy can produce a sustained release effect. Other compositions also include liposomally entrapped antibodies. Liposomes containing such antibodies are prepared by methods known per se: U.S. Pat. No. DE 3,218,121; Epstein et al., Proc. Natl. Acad. Sci. USA, (1985) 82:3688-3692; Hwang et al., Proc. Natl. Acad. Sci. USA, (1980) 77:4030-4034; EP 52,322; EP 36,676; EP 88,046; EP 143,949; 142,641; Japanese patent application 83-118008; U.S. Pat. Nos. 4,485,045 and 4,544,545; and EP 102,324.

The dosage of the antibody formulation for a given patient will be determined by the attending physician taking into consideration various factors known to modify the action of drugs including severity and type of disease, body weight, sex, diet, time and route of administration, other medications and other relevant clinical factors. Therapeutically effective dosages can be determined by either in vitro or in vivo methods.

An effective amount of the antibodies, described herein, to be employed therapeutically will depend, for example, upon the therapeutic objectives, the route of administration, and the condition of the patient. Accordingly, it is preferred for the therapist to titer the dosage and modify the route of administration as required to obtain the optimal therapeutic effect. A typical daily dosage might range from about 0.001 mg/kg to up to 100 mg/kg or more, depending on the factors mentioned above. Typically, the clinician will administer the therapeutic antibody until a dosage is reached that achieves the desired effect. The progress of this therapy is easily monitored by conventional assays or as described herein.

It will be appreciated that administration of therapeutic entities in accordance with the compositions and methods herein will be administered with suitable carriers, excipients, and other agents that are incorporated into formulations to provide improved transfer, delivery, tolerance, and the like. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures can be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol. Pharmacol. 32(2):210-8 (2000), Wang W. “Lyophilization and development of solid protein pharmaceuticals.” Int. J. Pharm. 203(1-2): 1-60 (2000), Charman W N “Lipids, lipophilic drugs, and oral drug delivery-some emerging concepts.” J Pharm Sci 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

Design and Generation of Other Therapeutics

In accordance with the present invention and based on the activity of the antibodies that are produced and characterized herein with respect to CD20, the design of other therapeutic modalities is facilitated and disclosed to one of skill in the art. Such modalities include, without limitation, advanced antibody therapeutics, such as bispecific antibodies, immunotoxins, radiolabeled therapeutics, and single antibody V domains, antibody-like binding agent based on other than V region scaffolds, generation of peptide therapeutics, gene therapies, particularly intrabodies, antisense therapeutics, and small molecules.

In connection with the generation of advanced antibody therapeutics, where complement fixation is a desirable attribute, it can be possible to sidestep the dependence on complement for cell killing through the use of bispecifics, immunotoxins, or radiolabels, for example.

For example, bispecific antibodies can be generated that comprise (i) two antibodies, one with a specificity to CD20 and another to a second molecule, that are conjugated together, (ii) a single antibody that has one chain specific to CD20 and a second chain specific to a second molecule, or (iii) a single chain antibody that has specificity to both CD20 and the other molecule. Such bispecific antibodies can be generated using techniques that are well known; for example, in connection with (i) and (ii) see e.g., Fanger et al. Immunol Methods 4:72-81 (1994) and Wright and Harris, supra and in connection with (iii) see e.g., Traunecker et al. Int. J. Cancer (Suppl.) 7:51-52 (1992). In each case, the second specificity can be made as desired. For example, the second specificity can be made to the heavy chain activation receptors, including, without limitation, CD16 or CD64 (see e.g., Deo et al. 18:127 (1997)) or CD89 (see e.g., Valerius et al. Blood 90:4485-4492 (1997)).

Antibodies can also be modified to act as immunotoxins utilizing techniques that are well known in the art. See e.g., Vitetta Immunol Today 14:252 (1993). See also U.S. Pat. No. 5,194,594. In connection with the preparation of radiolabeled antibodies, such modified antibodies can also be readily prepared utilizing techniques that are well known in the art. See e.g., Junghans et al. in Cancer Chemotherapy and Biotherapy 655-686 (2d edition, Chafner and Longo, eds., Lippincott Raven (1996)). See also U.S. Pat. Nos. 4,681,581, 4,735,210, 5,101,827, 5,102,990 (RE 35,500), 5,648,471, and 5,697,902. Each of immunotoxins and radiolabeled molecules would be likely to kill cells expressing the desired multimeric enzyme subunit oligomerization domain. In some embodiments, a pharmaceutical composition comprising an effective amount of the antibody in association with a pharmaceutically acceptable carrier or diluent is provided.

In some embodiments, an anti-CD20 antibody is linked to an agent (e.g., radioisotope, pharmaceutical composition, or a toxin). Preferably, such antibodies can be used for the treatment of diseases, such diseases can relate cells expressing CD20 or cells overexpressing CD20. For example, it is contemplated that the drug possesses the pharmaceutical property selected from the group of antimitotic, alkylating, antimetabolite, antiangiogenic, apoptotic, alkaloid, COX-2, and antibiotic agents and combinations thereof. The drug can be selected from the group of nitrogen mustards, ethylenimine derivatives, alkyl sulfonates, nitrosoureas, triazenes, folic acid analogs, anthracyclines, taxanes, COX-2 inhibitors, pyrimidine analogs, purine analogs, antimetabolites, antibiotics, enzymes, epipodophyllotoxins, platinum coordination complexes, vinca alkaloids, substituted ureas, methyl hydrazine derivatives, adrenocortical suppressants, antagonists, endostatin, taxols, camptothecins, oxaliplatin, doxorubicins and their analogs, and a combination thereof.

Examples of toxins further include gelonin, Pseudomonas exotoxin (PE), PE40, PE38, diphtheria toxin, ricin, ricin, abrin, alpha toxin, saporin, ribonuclease (RNase), DNase I, Staphylococcal enterotoxin-A, pokeweed antiviral protein, gelonin, Pseudomonas endotoxin, as well as derivatives, combinations and modifications thereof.

Examples of radioisotopes include gamma-emitters, positron-emitters, and x-ray emitters that can be used for localization and/or therapy, and beta-emitters and alpha-emitters that can be used for therapy. The radioisotopes described previously as useful for diagnostics, prognostics and staging are also useful for therapeutics. Non-limiting examples of anti-cancer or anti-leukemia agents include anthracyclines such as doxorubicin (adriamycin), daunorubicin (daunomycin), idarubicin, detorubicin, carminomycin, epirubicin, esorubicin, and morpholino and substituted derivatives, combinations and modifications thereof. Exemplary pharmaceutical agents include cis-platinum, taxol, calicheamicin, vincristine, cytarabine (Ara-C), cyclophosphamide, prednisone, daunorubicin, idarubicin, fludarabine, chlorambucil, interferon alpha, hydroxyurea, temozolomide, thalidomide, and bleomycin, and derivatives, combinations and modifications thereof. Preferably, the anti-cancer or anti-leukemia is doxorubicin, morpholinodoxorubicin, or morpholinodaunorubicin.

As will be appreciated by one of skill in the art, in the above embodiments, while affinity values can be important, other factors can be as important or more so, depending upon the particular function of the antibody. For example, for an immunotoxin (toxin associated with an antibody), the act of binding of the antibody to the target can be useful; however, in some embodiments, it is the internalization of the toxin into the cell that is the desired end result. As such, antibodies with a high percent internalization can be desirable in these situations. Thus, in one embodiment, antibodies with a high efficiency in internalization are contemplated. A high efficiency of internalization can be measured as a percent internalized antibody, and can be from a low value to 100%. For example, in varying embodiments, 0.1-5, 5-10, 10-20, 20-30, 30-40, 40-45, 45-50, 50-60, 60-70, 70-80, 80-90, 90-99, and 99-100% can be a high efficiency. As will be appreciated by one of skill in the art, the desirable efficiency can be different in different embodiments, depending upon, for example, the associated agent, the amount of antibody that can be administered to an area, the side effects of the antibody-agent complex, the type (e.g., cancer type) and severity of the problem to be treated.

In other embodiments, the antibodies disclosed herein provide an assay kit for the detection of CD20 expression in mammalian tissues or cells in order to screen for a disease or disorder associated with changes in expression of CD20. The kit comprises an antibody that binds CD20 and means for indicating the reaction of the antibody with the antigen, if present.

In some embodiments, an article of manufacture is provided comprising a container, comprising a composition containing an anti-CD20 antibody, and a package insert or label indicating that the composition can be used to treat disease mediated by CD20 expression. Preferably a mammal and, more preferably, a human, receives the anti-CD20 antibody.

Combinations

The anti-neoplastic treatment defined herein may be applied as a sole therapy or may involve, in addition to the compounds of the invention, conventional surgery, bone marrow and peripheral stem cell transplantations or radiotherapy or chemotherapy. Such chemotherapy may include one or more of the following categories of anti tumor agents:

(i) cytotoxic agents such as fludarabine, 2-chlorodeoxyadenosine, chlorambucil or doxorubicin and combination thereoff such as Fludarabine+cyclophosphamide, CVP: cyclophosphamide+vincristine+prednisone, ACVBP: doxorubicin+cyclophosphamide+vindesine+bleomycin+prednisone, CHOP: cyclophosphamide+doxorubicin+vincristine+prednisone, CNOP: cyclophosphamide+mitoxantrone+vincristine+prednisone, m-BACOD: methotrexate+bleomycin+doxorubicin+cyclophosphamide+vincristine+dexamethasone+leucovorin, MACOP-B: methotrexate+doxorubicin+cyclophosphamide+vincristine+prednisone fixed dose+bleomycin+leucovorin, or ProMACE CytaBOM: prednisone+doxorubicin+cyclophosphamide+etoposide+cytarabine+bleomycin+vincristine+methotrexate+leucovorin.

(ii) agents which inhibit cancer cell invasion (for example metalloproteinase inhibitors like marimastat and inhibitors of urokinase plasminogen activator receptor function);

(iii) inhibitors of growth factor or survival signaling function, for example such inhibitors include growth factor antibodies (for example antibodies directled against B-LyS), growth factor receptor antibodies (for example antibodies directled against CD40 or TRAIL receptors TRAILR1 and TRAILR2), farnesyl transferase inhibitors or tyrosine kinase inhibitors and serine/threonine kinase inhibitors, MEK inhibitors, inhibitors of survival signaling proteins such as Bcl-2, Bcl-XL for example ABT-737;

(iv) antiangiogenic agents such as those which inhibit the effects of vascular endothelial growth factor, (for example the anti vascular endothelial cell growth factor antibody bevacizumab [Avastin™], anti-vascular endothelial growth factor receptor antibodies such anti-KDR antibodies and anti-flt1 antibodies, compounds such as those disclosed in International Patent Applications WO 97/22596, WO 97/30035, WO 97/3285, WO 98/13354, WO00/47212 and WO01/32651) and compounds that work by other mechanisms (for example linomide, inhibitors of integrin avb3 function and angiostatin);

(v) vascular damaging agents such as Combretastatin A4 and compounds disclosed in International Patent Applications WO 99/02166, WO 00/40529, WO 00/41669, WO 01/92224, WO 02/04434 and WO 02/08213;

(vi) antisense therapies, for example those which are directed to the targets listed above, such as G-3139 (Genasense), an anti bcl2 antisense;

(vii) gene therapy approaches, including for example approaches to replace aberrant genes such as aberrant p53 or aberrant BRCA1 or BRCA2, GDEPT (gene directed enzyme pro drug therapy) approaches such as those using cytosine deaminase, thymidine kinase or a bacterial nitroreductase enzyme and approaches to increase patient tolerance to chemotherapy or radiotherapy such as multi drug resistance gene therapy; and

(viii) immunotherapy approaches, including for example treatment with Alemtuzumab (campath-1H™), a monoclonal antibody directed at CD52, or treatment with antibodies directed at CD22, ex vivo and in vivo approaches to increase the immunogenicity of patient tumour cells, transfection with cytokines such as interleukin 2, interleukin 4 or granulocyte macrophage colony stimulating factor, approaches to decrease T cell anergy such as treatment with monoclonal antibodies inhibiting CTLA-4 function, approaches using transfected immune cells such as cytokine transfected dendritic cells, approaches using cytokine transfected tumour cell lines and approaches using anti idiotypic antibodies.

(ix) inhibitor of protein degradation such as proteasome inhibitor such as Velcade (bortezomid).

(x) biotherapeutic therapeutic approaches for example those which use peptides or proteins (such as antibodies or soluble external receptor domain constructions) which either sequest receptor ligands, block ligand binding to receptor or decrease receptor signalling (e.g. due to enhanced receptor degradation or lowered expression levels)

In one embodiment of the invention the anti-neoplastic treatments of the invention are combined with agents which inhibit the effects of vascular endothelial growth factor (VEGF), (for example the anti-vascular endothelial cell growth factor antibody bevacizumab (Avastin®), anti-vascular endothelial growth factor receptor antibodies such anti-KDR antibodies and anti-flt1 antibodies, compounds such as those disclosed in International Patent Applications WO 97/22596, WO 97/30035, WO 97/3285, WO 98/13354, WO00/47212 and WO01/32651) and compounds that work by other mechanisms (for example linomide, inhibitors of integrin avb3 function and angiostatin); In another embodiment of the invention the anti-angiogenic treatments of the invention are combined agents which inhibit the tyrosine kinase activity of the vascular endothelial growth factor receptor, KDR (for example AZD2171 or AZD6474). Additional details on AZD2171 may be found in Wedge et al (2005) Cancer Research. 65(10):4389-400. Additional details on AZD6474 may be found in Ryan & Wedge (2005) British Journal of Cancer. 92 Suppl 1:S6-13. Both publications are herein incorporated by reference in their entireties. In another embodiment of the invention, the fully human antibodies 1.1.2, 1.5.3, 2.1.2 are combined alone or in combination with Avastin™, AZD2171 or AZD6474.

Such conjoint treatment may be achieved by way of the simultaneous, sequential or separate dosing of the individual components of the treatment. Such combination products employ the compounds of this invention, or pharmaceutically acceptable salts thereof, within the dosage range described hereinbefore and the other pharmaceutically active agent within its approved dosage range.

### EXAMPLES

The following examples, including the experiments conducted and results achieved are provided for illustrative purposes only and are not to be construed as limiting upon the teachings herein.

### Example 1

### Immunization and Titering

Cloning of human CD20 Plasmid

Total RNA was isolated from RAJI cells using RNAzol B RNA isolation solution (Tel-Test, INC, Friendswood, Tex.) according to the manufacturer's instructions and quantitated by ultraviolet absorption at 260 nm (Bio-RAD smartspec™ 3000). Two micrograms of total RNA was random primed with single stranded cDNA synthesis kit (GIBCO-BRL) according to the manufacturer's instructions. Single stranded cDNA was amplified using Taq DNA polymerase (QIAGEN, Valencia, Calif.) with oligonucleotide primers (Operon, Huntsville, Ala.) as follows:

| | |
|---|---|
| Forward primer: | |
| 5′-TCAGGAGTTTTGAGAGCAAAATG-3′ | (SEQ ID NO. 137) |
| and | |
| Reverse primer: | |
| 5′-AACAGAAGAAATCACTTAAGGAG-3′. | SEQ ID NO. 138) |

The PCR conditions were as follows: an initial denaturation at 94° C. for 5 minute, 30 cycles of 94° C. for 30 seconds, 55° C. for 45 seconds, 72° C. for 1 minute and extension one cycle for 10 minutes at 72° C.

PCR products were resolved by agarose gel electrophoresis, isolated using Qiaquick gel extraction kit (QIAGEN, Valencia, Calif.) and ligated with T4 ligase (New England Biolabs, Beverly, Mass.) into pCR 3.1 bidirectional eukaryotic TA expression vector (Invitrogen, Carlsbad, Calif.). Top10F′ Escherichia coli strain was used for transformation. Clones resistant to ampicillin were propagated in bacteria and evaluated for the presence of the 1 kb insert by digestion with EcoRI (New England Biolabs, Beverly, Mass.). All PCR amplification products were sequenced to assure correct DNA sequences using BigDye terminators method with 3100 Genetic Analyzer (PE Biosystems, Foster City, Calif.).

Cells and Transfection

HEK 293F and CHO K1 cells were grown in DMEM/F12 (50/50 mix) media supplemented with 10% FBS, 2 mM L-Glutamine, 50 μM BME, 100 units Penicillin-g/ml, 100 units MCG Streptomycin/ml. A human CD20/pCR3.1 plasmid was transfected into HEK 293F and CHO K1 cells using LipofectAMINE 2000 Reagent (Invitrogen, Carlsbad, Calif.), according to the manufacturer's instructions. Transfection proceeded for 48 hours followed by selection with 1 mg/ml G418 (Invitrogen, Carlsbad, Calif.) for two weeks. Stable G418 resistant clones were stained with primary mouse anti-human CD20 monoclonal antibody (BD) followed by PE conjugated goat anti-mouse IgG (CalTag Laboratories, Burlingame, Calif.) and analyzed by FACS with FACS Vantage (BD, Franklin Lakes, N.J.).

Immunization

CD20 expressed in human cancer cell lines Ramos, Daudi and CD20-CHO cells were used as an antigen. Monoclonal antibodies against CD20 were developed by sequentially immunizing XenoMouse® mice (XenoMouse strains: XM3B3:IgG1K, XM3B3L3:IgG1KL, XM3B3L:IgG1L, XM3C-1: IgG4K, XM3C-1L3:IgG4KL, and XM3C-1L: IgG4L, Abgenix, Inc. Fremont, Calif.). XenoMouse animals were immunized via footpad route for all injections by conventional means. The total volume of each injection was 50 μl per mouse, 25 μl per footpad.

### Example 2

### Recovery of Lymphocytes, B-Cell Isolations, Fusions and Generation of Hybridomas

Selected immunized mice were sacrificed by cervical dislocation and the draining lymph nodes were harvested and pooled from each cohort. The lymphoid cells were dissociated by grinding in DMEM to release the cells from the tissues, and the cells were suspended in DMEM. The cells were counted, and 0.9 ml DMEM per 100 million lymphocytes was added to the cell pellet to resuspend the cells gently but completely. Using 100 μl of CD90+ magnetic beads per 100 million cells, the cells were labeled by incubating the cells with the magnetic beads at 4° C. for 15 minutes. The magnetically-labeled cell suspension containing up to 10⁸ positive cells (or up to 2×10⁹ total cells) was loaded onto an LS+ column and the column washed with DMEM. The total effluent was collected as the CD90-negative fraction (most of these cells were expected to be B cells).

A fusion was performed by mixing washed enriched B cells from above and nonsecretory myeloma P3X63Ag8.653 cells purchased from ATCC (cat. no. CRL 1580) (Kearney et al, J. Immunol. 123, 1979, 1548-1550) at a ratio of 1:1. The cell mixture was gently pelleted by centrifugation at 800×g. After complete removal of the supernatant, the cells were treated with 2-4 mL of Pronase solution (CalBiochem, cat. # 53702; 0.5 mg/mL in PBS) for no more than 2 minutes. Then 3-5 ml of FBS was added to stop the enzyme activity and the suspension was adjusted to 40 mL total volume using electro cell fusion solution, ECFS (0.3 M Sucrose, Sigma, Cat# S7903, 0.1 mM Magnesium Acetate, Sigma, Cat# M2545, 0.1 mM Calcium Acetate, Sigma, Cat# C4705). The supernatant was removed after centrifugation and the cells were resuspended in 40 mL ECFS. This wash step was repeated and the cells again were resuspended in ECFS to a concentration of 2×10⁶ cells/mL.

Electro-cell fusion was performed using a fusion generator, model ECM2001, Genetronic, Inc., San Diego, Calif. The fusion chamber size used was 2.0 mL, using the following instrument settings: Alignment condition: voltage: 50 V, time: 50 seconds; membrane breaking at: voltage: 3000 V, time: 30 μseconds; post-fusion holding time: 3 seconds.

After ECF, the cell suspensions were carefully removed from the fusion chamber under sterile conditions and transferred into a sterile tube containing the same volume of Hybridoma Culture Medium (DMEM (JRH Biosciences), 15% FBS (Hyclone), supplemented with L-glutamine, pen/strep, OPI (oxaloacetate, pyruvate, bovine insulin) (all from Sigma) and IL-6 (Boehringer Mannheim). The cells were incubated for 15-30 minutes at 37° C., and then centrifuged at 400×g (1000 rpm) for five minutes. The cells were gently resuspended in a small volume of Hybridoma Selection Medium (Hybridoma Culture Medium supplemented with 0.5×HA (Sigma, cat. # A9666)), and the volume was adjusted appropriately with more Hybridoma Selection Medium, based on a final plating of 5×10⁶ B cells total per 96-well plate and 200 μL per well. The cells were mixed gently and pipetted into 96-well plates and allowed to grow. On day 7 or 10, one-half the medium was removed, and the cells were re-fed with Hybridoma Selection Medium.

### Example 3

### Selection of Candidate Antibodies by FMAT and FACS

After 14 days of culture, hybridoma supernatants were screened for CD20-specific monoclonal antibodies by Fluorometric Microvolume Assay Technology (FMAT) by screening against recombinant CHO-human CD20 transfectant cells and counter-screening against parental CHO cells.

The culture supernatants from the positive hybridoma cells growth wells based on primary screen were removed and the CD20 positive hybridoma cells were suspended with fresh hybridoma culture medium and were transferred to 24-well plates. After two days in culture, these supernatants were ready for a secondary confirmation screen. In the secondary confirmation screen, the positives in the first screening were screened by FACS with two sets or three sets of detection antibodies used separately: 1.25 ug/ml GAH-Gamma Cy5 (JIR#109-176-098) for human gamma chain detection; 1.25 ug/ml GAH-Kappa PE (S.B.#2063-09) for human kappa light chain detection and 1.25 ug/ml GAH-lambda PE (S.B.#2073-09) for human lambda light chain detection in order to confirm that the anti-CD20 antibodies were fully human.

78 fully human IgG/kappa or IgG/lambda CD20 specific monoclonal antibodies were generated.

TABLE 2
FULLY HUMAN CD20 SPECIFIC MONOCLONAL ANTIBODIES

| | | | | CD20 | |
|---|---|---|---|---|---|
| | | | | antigen specific | |
| | | | | antibody | immuni- |
| | | | | (FACS | zation |
| Cohort | Strain | Antigen | FMAT | confirmed) | time |
| 1 | G1k | Ramos | 77 | 13 | 27-38 days |
| 2 | G1KL | Ramos | 29 | 4 | |
| 3 | G4K | Ramos | 26 | 7 | |
| 4 | G4KL | Ramos | 18 | 7 | |
| 5 | G1K | CHO-CD20 | 94 | 0 | 27-38 days |
| 6 | G1L | CHO-CD20 | 25 | 3 | |
| 7 | G4K | CHO-CD20 | 85 | 28 | |
| 8 | G4L | CHO-CD20 | 50 | 2 | |
| 9 | G1K | CHO-CD20 | 32 | 0 | 67 days |
| 10 | G1K | Daudi | 165 | 14 | 59 days |

### Example 4

### Apoptotic Activity

Two experimental approaches were implemented to screen and identify antibody lines exhibiting pro-apoptotic activity in the Ramos human lymphoma cell line. More specifically, apoptotic activity was evaluated using the CellTiterGlo assay and by Propidium Iodide/Hoechst staining in conjunction with an automated fluorescent microscope.

In brief, for the CelITiterGlo assay (Promega, Madison, Wis.), Ramos cells were obtained from the ATCC and were maintained in RPMI medium supplemented with 10% FBS, 1% sodium pyruvate, and 1% HEPES buffer. Cells were seeded at a concentration of 100,000 cells/ml (100 μl/well) in 96-well plates. Cells were incubated at 37° C. and 5% CO₂ for 72 hours. The assay was terminated 72 hours post addition of the antibodies and performed per instructions provided in the kit. Cells were treated with antibody hybridoma supernatant at a concentration of either 1 or 10 μg/ml in the presence or absence of secondary cross-linking antibody. Isotype (IgG1 and IgG4) as well as Rituxan® (Rituximab—Genentech Inc.) and B1 (Beckman Coulter, Miami, Fla.) antibodies were used as controls. (Note: Dialysis was performed on the B1 antibody to remove sodium azide from the stock buffer solution (0.10% sodium azide).) The determination of percent survival for the treatment samples was based on normalizing the control sample (i.e. no treatment) to 100% viable.

For the Propidium Iodide/Hoechst staining study, cells were seeded at a concentration of 100,000 cells/ml (50 ul/well) in a 96-well plate. Cells were incubated at 37° C. and 5% CO₂ for 48 hours. Cells were treated with antibody hybridoma supernatant (purified over a Protein A Sepharose column followed by dialysis in PBS) in a titration with concentrations ranging from 5000 ng/ml to 8 ng/ml. All experiments were run in duplicate in the presence (N=2) or absence (N=1) of secondary cross-linking antibody. Isotype (IgG1 and diluent controls) as well as Rituximab and B1 antibodies were used as controls. After the 48-hour incubation, cells were stained with PI/Hoechst and visualized using an automated fluorescent microscope. Percent apoptosis was determined by taking a ratio of the number of PI positive cells (apoptotic) versus the number of Hoechst positive cells (total).

For the CellTiterGlo analysis, cells were plated in duplicate and standard error of mean was determined using Excel software. For the Propidium Iodide/Hoechst staining analysis, average values were generated from duplicate points. These averages were used to generate a dose response curve and these curves were compared to isotype and diluent controls to assess apoptotic activity.

In summary, this study interrogated a net total of 25 hybridoma supernatants. Analysis revealed that a majority of the anti-CD20 antibody hybridoma lines exhibited apoptotic activity in the aforementioned assays. A total of 22 lines were selected for cloning.

TABLE 3
SUMMARY OF ANTI-CD20 HYBRIDOMA LINE SUPERNATANTS
EVALUATED IN APOPTOSIS ASSAYS
Table lists the negative lines from each assay.
Lines indicated in bold were consistently negative and not carried forward.

| | Study #1/Hoechst apoptosis assay on | | | Study #2/CellTiterGlo apoptosis | | |
|---|---|---|---|---|---|---|
| | Ramos cells | | | assay on Ramos cells | | |
| | with | with | without | with | with | without |
| Lines | CrossLinker | CrossLinker | CrossLinker | CrossLinker | CrossLinker | CrossLinker |
| Evaluated: | (duplicates) | (duplicates) | (duplicates) | (singlicates) | (duplicates) | (duplicates) |
| 1.1 (G1) | | | | | | |
| 1.2 (G1) | | | | | 1.2 | 1.2 |
| 1.3 (G1) | | | | | | |
| 1.4 (G1) | | | | | | 1.4 |
| 1.5 (G1) | | | | | | |
| 1.6 (G1) | | | | | | 1.6 |
| 1.9 (G1) | | | | | | 1.9 |
| 1.10 (G1) | | | | | | |
| 1.11 (G1) | | | | | | |
| 1.12 (G1) | | | | | | |
| 1.13 (G1) | 1.13 | | | | | |
| 2.1 (G1) | | | | | | |
| 2.2 (G1) | | | 2.2 | | | |
| 2.4 (G1) | | | | | | |
| 3.1 (G4) | | | 3.1 | | | |
| 3.2 (G4) | 3.2 | 3.2 | 3.2 | | | |
| 3.3 (G4) | 3.3 | 3.3 | 3.3 | | | |
| 3.4 (G4) | | | 3.4 | | | |
| 3.6 (G4) | 3.6 | 3.6 | 3.6 | 3.6 | 3.6 | |
| 3.7 (G4) | | | | | | |
| 4.1 (G4) | 4.1 | 4.1 | 4.1 | 4.1 | 4.1 | |
| 4.2 (G4) | | | 4.2 | | | 4.2 |
| 4.5 (G4) | | | 4.5 | | | |
| 4.6 (G4) | 4.6 | 4.6 | 4.6 | | | |
| 4.7 (G4) | 4.7 | 4.7 | 4.7 | 4.7 | 4.7 | 4.7 |

TABLE 4
SUMMARY OF ANTI-CD20 HYBRIDOMA LINE SUPERNATANTS
TESTED NEGATIVE IN APOPTOSIS ASSAYS

| | | Dual Negative |
|---|---|---|
| Study #1 Apoptosis | Study #2 Apoptosis | (These clones were not carried |
| Assay Negatives | Assay Negatives | forward to cloning) |
| 3.2 | 1.2 | |
| 3.3 | | |
| 3.6 | 3.6 | 3.6 |
| 4.1 | 4.1 | 4.1 |
| 4.6 | | |
| 4.7 | 4.7 | 4.7 |

It should be realized that the above-described process produces an oligoclonal mixture where the number of hybridoma lineages present in each sample varies from one to several to many. There is likely one CD20-specific antibody lineage per mixture of hybridomas in each well. Additionally, the actual antigen-specific cells in the oligoclonal mix can vary widely in their productivity (the amount of antibody they produce and secrete). A strong signal in an assay can be the result of a high concentration of antibody, an antibody with a high affinity for the target, or a combination of these factors. Thus, while quantitative results are presented from these assays, these results are interpreted in a “positive” versus “negative” way, looking at the several data points obtained and comparing them to controls.

Cloning was initiated on all lines recovered from fusions 1 and 2 (where the antibody was IgG1). Lines from fusions 3 and 4 were also cloned with the exception of lines 3.6, 4.1, and 4.7.

### Example 5

### Apoptosis Assay: CellTiterGlo Viability Assay without Cross-Linker

To determine the amount of ATP present in the cell, which correlates with the number of viable cells present, CellTiterGlo assays were performed. Briefly, lymphoma (Ramos) cells were plated into a Costar 96-well flat bottom plate (Catalog # 3603) at 10,000 cells per well in a volume of 50 μl. Primary antibodies were added as 25 μl/well in tissue culture media and allowed to incubate with cells for 10 minutes at room temperature. Following incubation, CellTiter Glo reagent (Promega Catalog #G7571) was added to cells and allowed to incubate for 10 minutes at room temperature in the dark. Plates were read per protocol instructions. Results are shown in FIG. 1 (plate 1 of 2 at 72 hours) and 2 (plate 2 of 2 at 72 hours), and summarized in Tables 5 and 6 below. Bolded values indicate EC₅₀ values that were superior to the Rituximab control.

### Example 6

### Apoptosis Assay: Alamar Blue Viability Assay without Cross-Linker

To measure apoptosis in Ramos cells, Alamar Blue (Biosource, Camarillo, Calif.) viability assays were performed. Alamar Blue is a redox indicator that changes color in response to metabolic activity. The internal environment of proliferating cells is more reduced than that of non-proliferating cells; Alamar Blue is reduced in proliferating cells and is accompanied by a measurable shift in color.

Briefly, Ramos cells were plated into Costar 96-well flat bottom plates (cat. no. 3603) at 10,000 cells/501. Primary antibody samples were added as 50 μl/well in tissue culture media and allowed to incubate at 37° C. for 48 hours. 10 μl per well of Alamar Blue dye was added and allowed to incubate overnight at 37° C. Following incubation, fluorescence was measured using Victor (Perkin Elmer, Wellesley, Mass.).

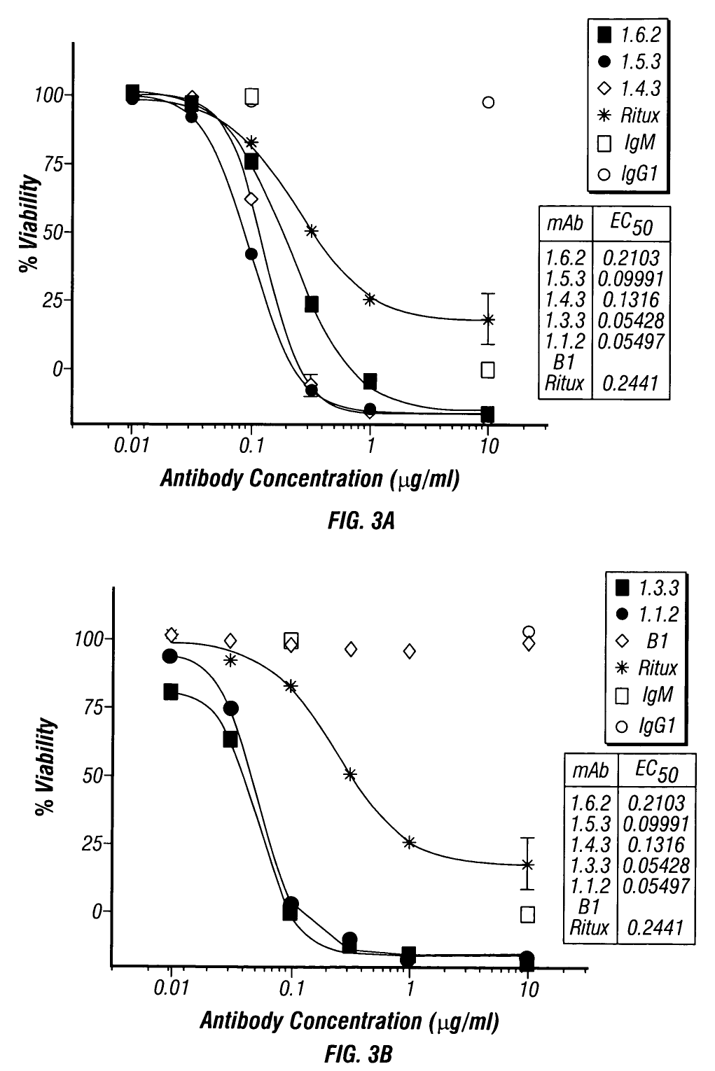

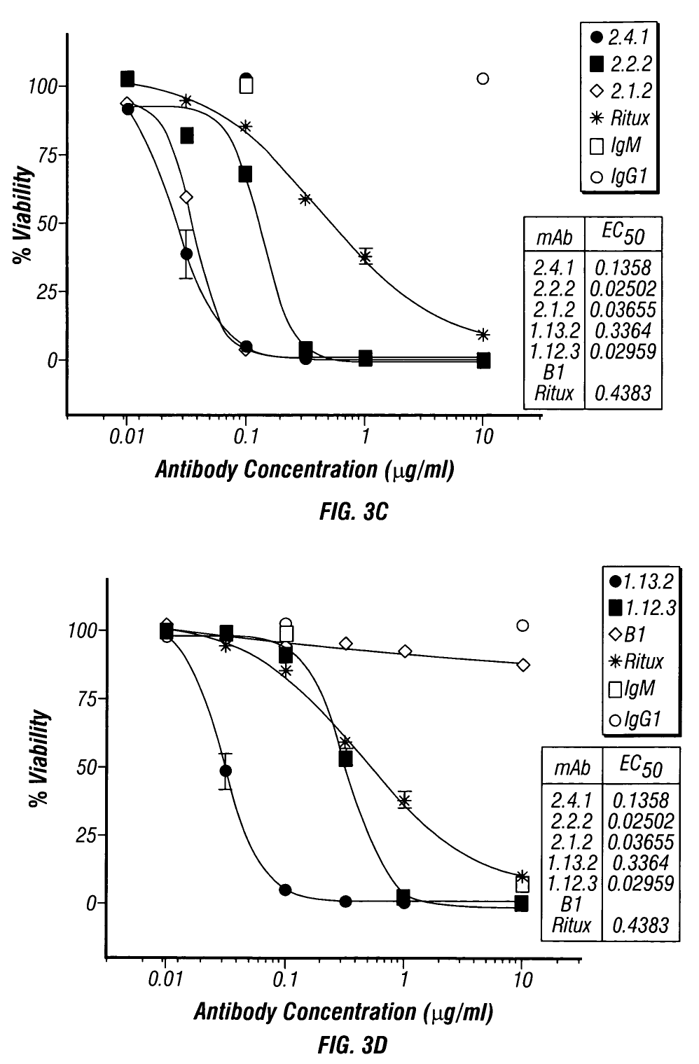

Results are shown in FIG. 3A through 3D (at 72 hours), and summarized in Tables 5 and 6 below. Bolded values indicate EC₅₀ values that were superior to the Rituximab control.

### Example 7

### Apoptosis Assay: WST-1 Viability Assay without Cross-Linker

To measure apoptosis in Ramos cells, WST-1 (Roche Molecular Biochemicals, Indianapolis, Ind.) viability assays were performed. WST-1 reduction assay is a colorimetric assay for quantification of cytotoxicity, based on cleavage of the WST-1 tetrazolium salt by mitochondrial deyhdrogenases in viable cells.

Briefly, Ramos cells were harvested, counted and resuspended in complete RPMI growth medium at a concentration of 66,667 cells/ml. Cells were plated in 50 μl (10,000 cells/well) in flat bottom plates (Costar, cat. no. 3595). Antibody was added to target cells at appropriate concentration as 50 μl/well and allowed to incubate for 68 hours at 37° C. 10 μl WST-1 (Roche 1 644 807) per well was added and incubated for 4 hours at 37° C. Plates were placed on a shaker for 1 minute and read at 450 nm.

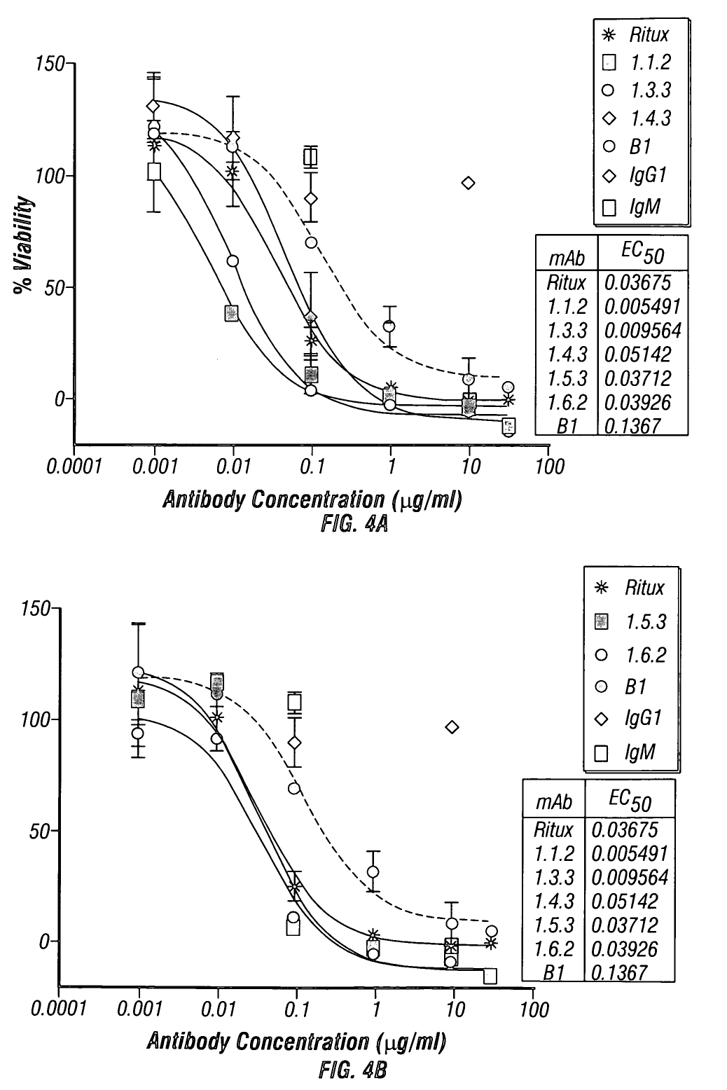

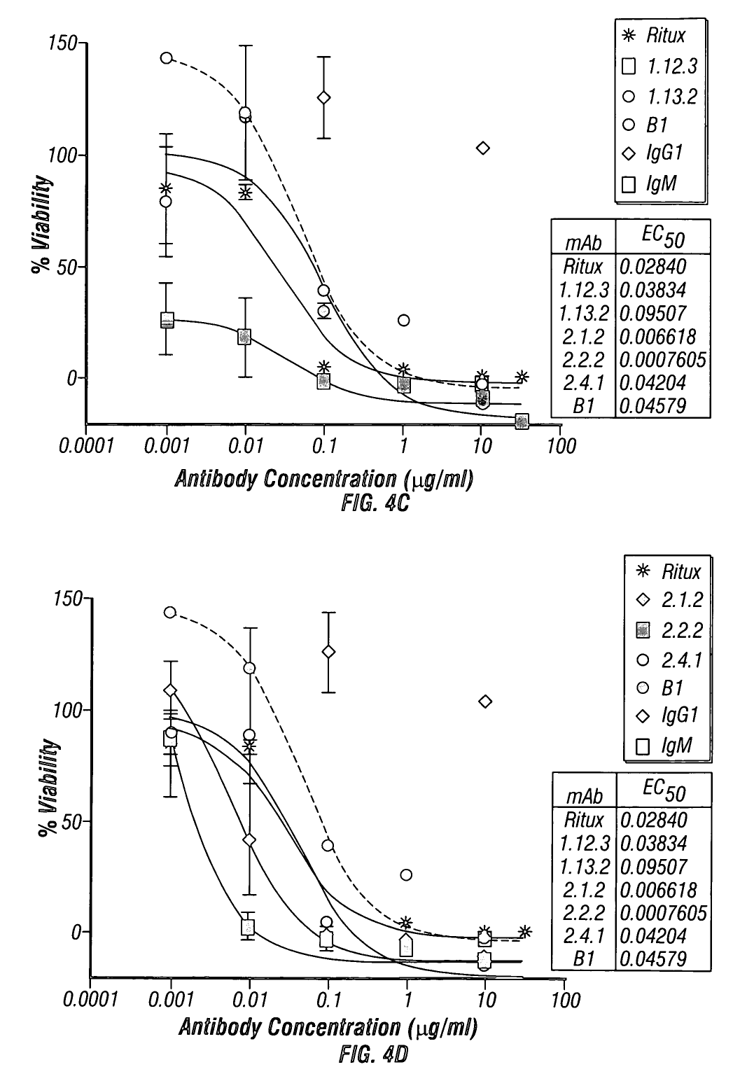

Results are shown in FIGS. 4A through 4D, and summarized in Tables 5 and 6 below. Bolded values indicate EC₅₀ values that were superior to the Rituximab control.

### Example 8

### Apoptosis Assay: Annexin V/PI Apoptosis Assay without Cross-Linker

Lymphoma cells were plated into Costar 96-well flat bottom plates (Catalog # 3603) at 200,000 cells per well in a volume of 50 μl. Primary antibodies were added as 25 μl/well in tissue culture media and allowed to incubate with cells for 10 minutes at room temperature. Following incubation, plates were centrifuged for 5 minutes at 1,200 rpm and the supernatant aspirated. Cell pellets were resuspended in 100 μl FACS buffer (2% FBS in 1×PBS) and plates were centrifuged for 5 minutes at 1,200 rpm. Pellets were resuspended in 100 μl of incubation buffer (95% 1× binding buffer, 2.5% Annexin V, 2.5% PI) and incubated for 10 to 15 minutes at room temperature in the dark. Following incubation, the volume in titer tubes was raised to 300 μl by adding 200 μl of 1× Buffer (w/o Annexin V and PI). Analysis was performed using channels FL-1 (Annexin V) and FL-3 (PI) with FACS Calibur.

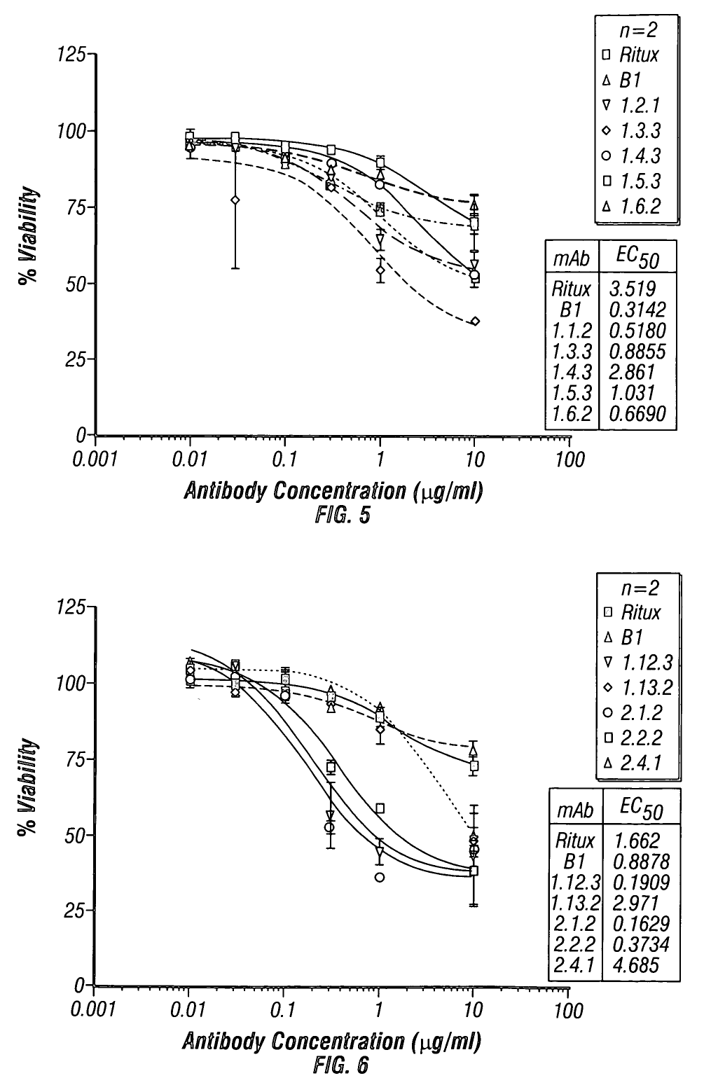

Results are shown in FIGS. 5 (plate 1 of 2 at 24 hours) and 6 (plate 2 of 2 at 24 hours), and summarized in Tables 5 and 6 below. Bolded values indicate EC₅₀ values that were superior to the Rituximab control.

### Example 9

### CDC Assay

Lymphoma cells (Ramos, Raji, or Daudi) were plated into Costar 96-well flat bottom plates (Catalog # 3603) at 100,000 cells per well in a volume of 25 μl. Primary antibody samples were added as 25 μl/well in tissue culture media and allowed to incubate at room temp for 10 minutes. Normal human serum was added at a concentration between 10 to 50% and diluted with growth media (serum concentration was titrated) (serum obtained from Advanced Research Technologies, San Diego, Calif.) and allowed to incubate at 37° C. for 1 hour. CellTiter Glo reagent (Promega Catalog # G7571) was added to cells and allowed to incubate for 10 minutes at room temperature in the dark. Plates were read per protocol instructions.

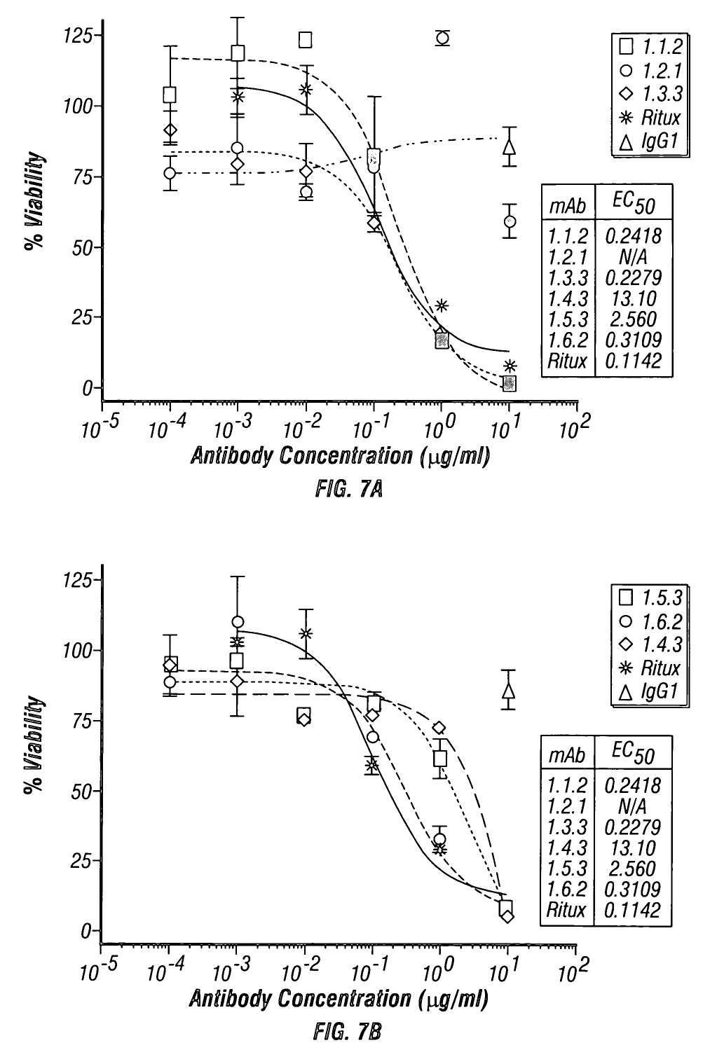

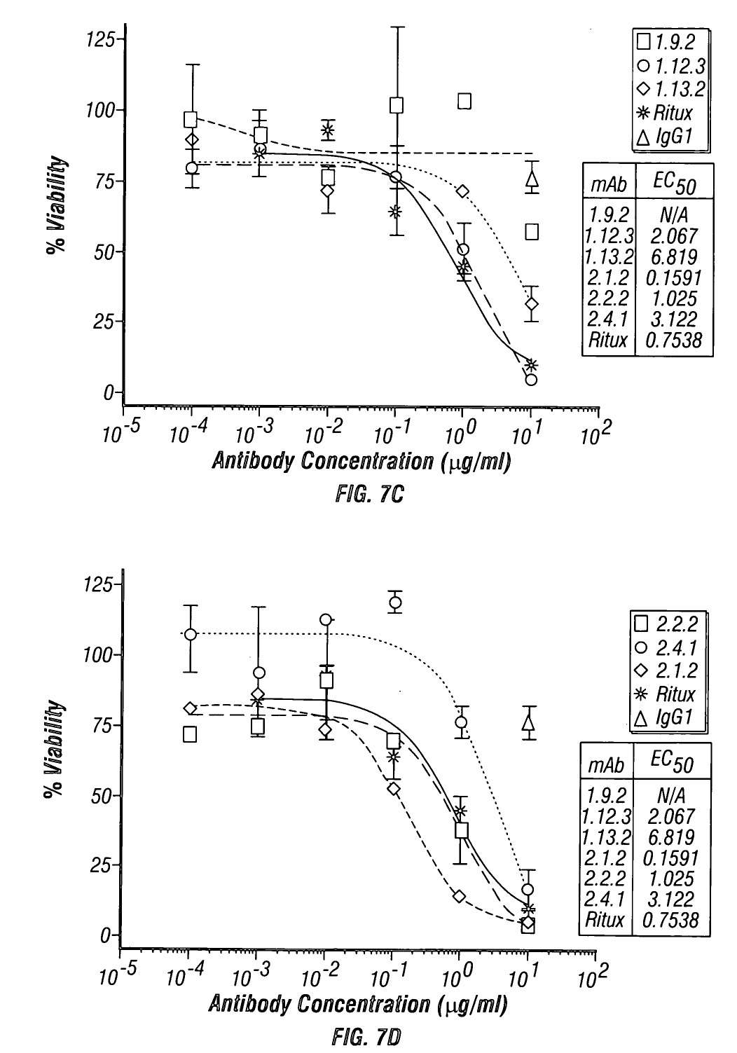

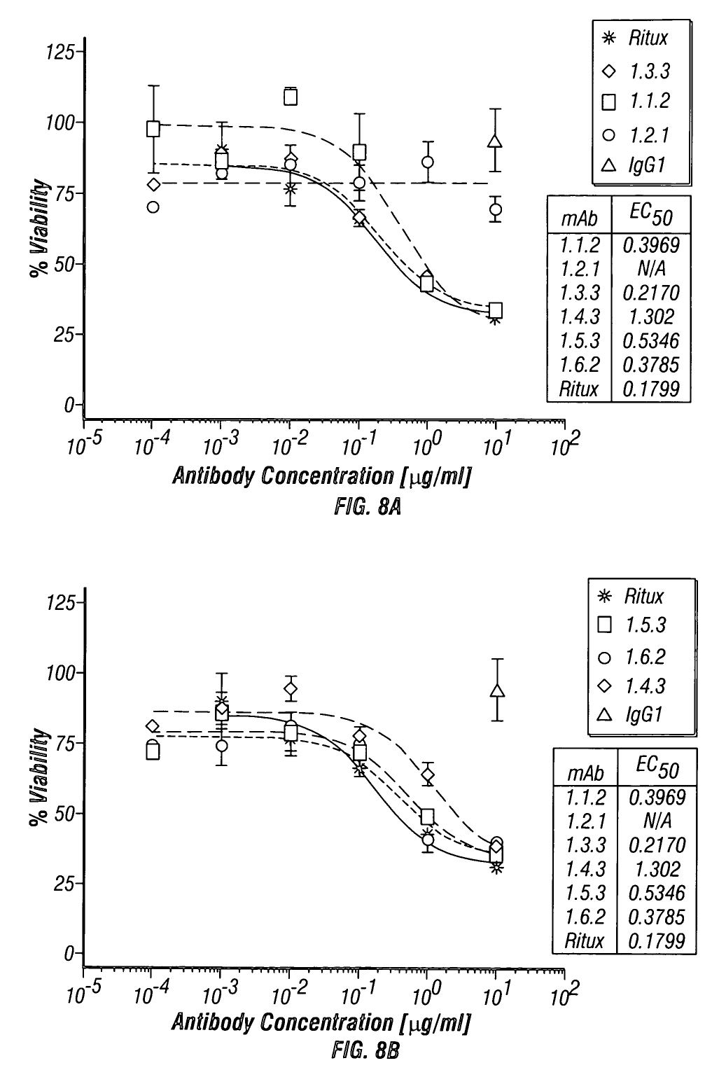

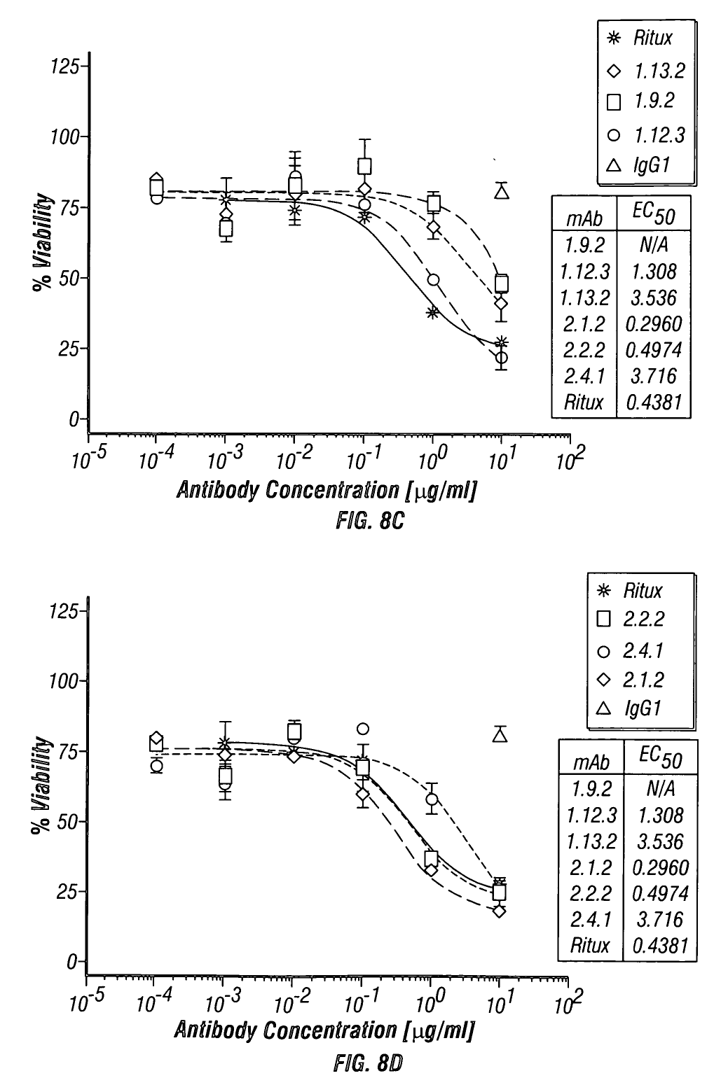

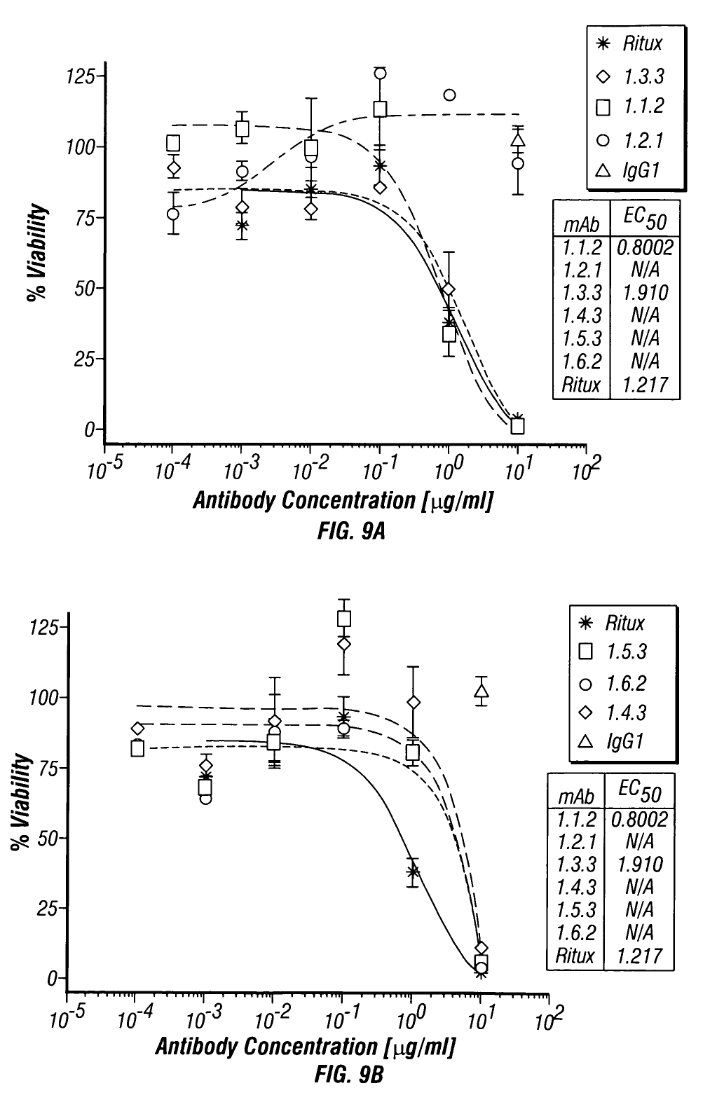

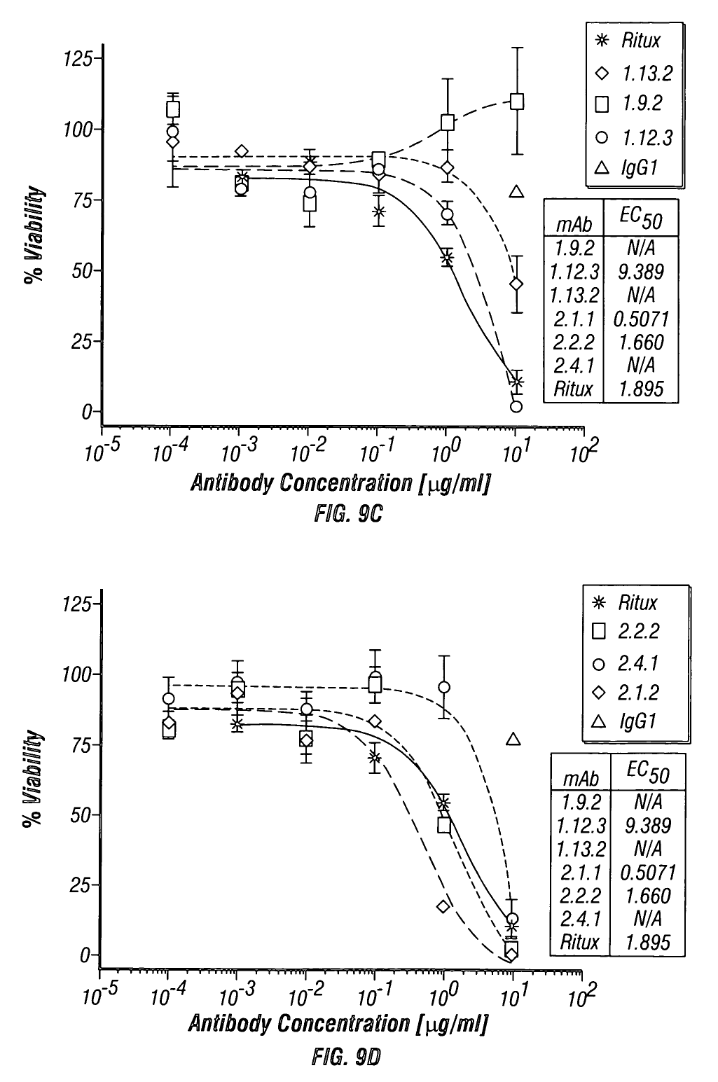

Results are shown in FIGS. 7A-7D (Ramos cell line at 1 hour), 8A-8D (Raji cell line at 1 hour), and 9A-9D (Daudi cell line at 1 hour). For all assays n=2, except Rituximab and IgG1 n=3. Average EC₅₀ values are shown in Tables 5 and 6 below. Bolded values indicate EC₅₀ values that were superior to the Rituximab control.

### Example 10

### ADCC of Human Anti-CD20 Antibodies

Isolation of PBMCs from Whole Blood (35-45 ml)

NK enrichment from PMBCs was performed using RosetteSep® Human NK Cell Enrichment cocktail and protocol (Cat. no. 15065). The RosetteSep® antibody cocktail crosslinks unwanted cells in human whole blood to multiple (RBCs), forming Immunorosettes. This increases the density of unwanted (rosetted) cells, such that they pellet along with the free RBCs when centrifuged over a buoyant density medium such as Ficol-Paque®. Desired cells are never between the plasma and the buoyant density medium.

Briefly, whole blood from donors was collected in heparinized or EDTA coated tubes and incubated with 2.25 ml RosetteSep® Human NK Cell Enrichment cocktail (Cat. no. 15065) for 20 minutes at room temperature per RosetteSep® protocol. Samples were then diluted with equal volume of PBS+2% FBS and 30 mL blood mixture was layered over 15 mL Ficoll (Amersham 17-1440-02) in 50 mL conical tubes. Tubes were centrifuged at 2150 RPM (tabletop centrifuge) with brake OFF for 30 minutes at room temperature. The interface layer was transferred to 2 clean 50 ml conical tubes. PBS+2% FBS was added to 50 ml and centrifuged for 10 minutes at 1200 RPM in a tabletop centrifuge (Beckman Allegra 6) with brake ON. Supernatants were discarded and pellets were resuspended in 1 ml PBS and stored on ice. Cells were counted using a hemacytometer and NK cells/ml in solution was determined [(total cells/#quadrants)*10e4*dilution factor].

Labeling of Tumor Target Cells with Calcein-AM

Calcein-AM is the cell-permeable version of calcein. It readily passes through the cell membrane of viable cells because of the enhanced hydrophobicity as compared to calcein. When Calcein-AM permeates into the cytoplasm, it is hydrolyzed by esterases in cells to calcein that is well retained inside of the cell. Thus, Calcein-AM is a suitable probe for staining viable cells. Viability assays using calcein are reliable and correlate well with the standard 51Cr-release assay.

Briefly, tumor target cells (Ramos, Raji, and Daudi) were harvested and resuspended in media at 1×10⁶ cells/ml. Calcein-AM (Sigma C1359) was added to final concentration of 10 μM (5 μl in 2 mL cells). Cells were incubated for 45 minutes at 37° C. Cells were then spun at 1200 RPM for 10 minutes, supernatants discarded, and pellets resuspended in fresh growth media (2×). Pellets were resuspended to 10,000 cells/75 μl. Target cells were plated in 75 μl (10,000 cells/well) in round bottom plates (Costar, cat. no. 3799). Antibodies were then added to target cells at appropriate concentrations as 50 μl/well diluted in media and allowed to incubate for 30 minutes at room temperature. Following incubation, 75 μl of effector cells were added at 100,000 cells/well and allowed to incubate for 4 hours at 37° C. Following incubation, plates were spun at 1200 RPM for 5 minutes. 100 μL supernatants were transferred to flat, black, clear bottom plates (Costar, cat. no. 3603) and fluorescence measured.

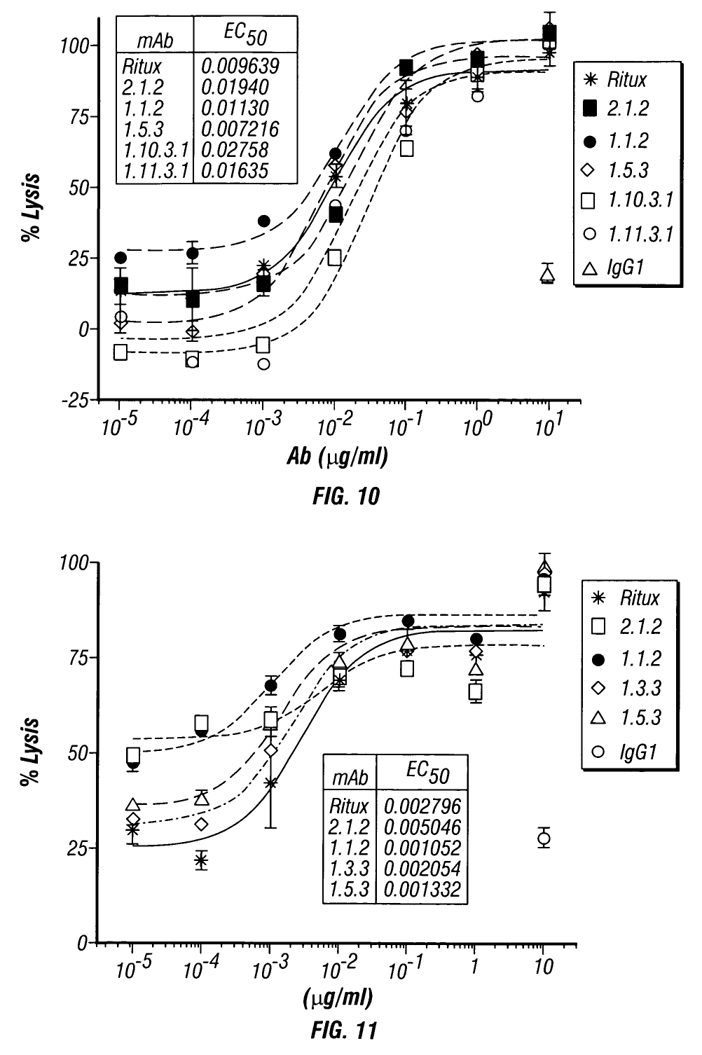

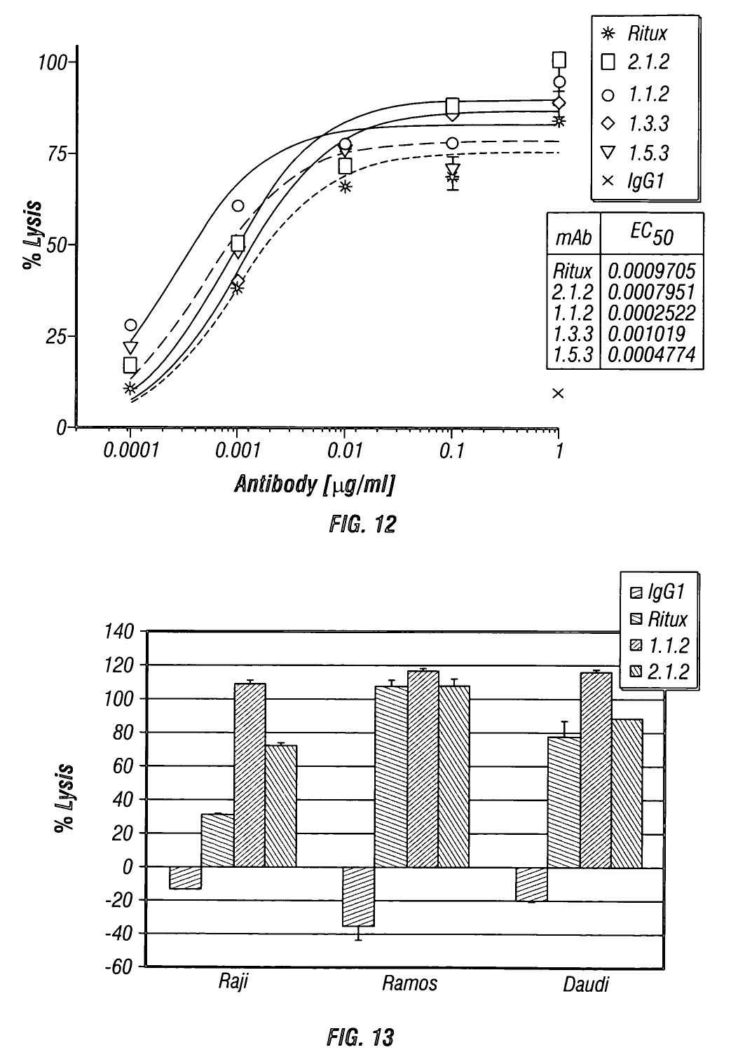

Results are shown in FIGS. 10-12, and summarized in Tables 5 and 6 below. Bolded values indicate EC₅₀ values that were superior to the Rituximab control.

Lead candidates were selected based on the number of instances where the test antibody exhibited superior potency as compared to controls in apoptosis, CDC, and ADCC. Anti-CD20 mAbs 2.1.2, 1.1.2, and 1.5.3 (note: mAbs 1.5.3 and 1.3.3 were found to have identical amino acid sequences) were identified as the top three candidates.

TABLE 5
ASSAY SUMMARY

| | | EC₅₀ | EC₅₀ | | EC₅₀ | | | | | | | | | |
|---|---|---|---|---|---|---|---|---|---|---|---|---|---|---|
| | | μg/ml | μg/ml | EC₅₀ | μg/ml | | | | | % Lysis | | % Lysis | | % Lysis |
| | # times | Ramos | Ramos | μg/ml | Ramos | EC₅₀ | EC₅₀ | EC₅₀ | % Lysis | Raji | % Lysis | Ramos | % Lysis | Daudi |
| | better | Apoptosis | Apoptosis | Ramos | Apoptosis | μg/ml | μg/ml | μg/ml | Raji | 0.001 | Ramos | 0.001 | Daudi | 0.001 |
| | than | Cell Titer | Alamar | Apoptosis | Annexin | Raji | Ramos | Daudi | 1 μg/ml | μg/ml | 1 μg/ml | μg/ml | 1 μg/ml | μg/ml |
| Ab | Ritux | Glo | Blue | WST-1 | V | CDC | CDC | CDC | ADCC | ADCC | ADCC | ADCC | ADCC | ADCC |
| Ritux | | 0.5755 | 0.2220 | 0.0326 | 2.59 | 0.31 | 0.43 | 1.56 | 62 | 16 | 78 | 5 | 95 | 33 |
| 1.1.2 | 10 | 0.041 | 0.0481 | 0.0055 | 0.518 | 0.40 | 0.24 | 0.80 | 53 | 12 | 84 | 15 | 96 | 77 |
| 1.2.1 | 2 | 15.22 | | | | >10 | >10 | >10 | 45 | 8 | 67 | 8 | 86 | 56 |
| 1.3.3 | 8 | 0.3323 | 0.0485 | 0.0096 | 0.886 | 0.22 | 0.23 | 1.91 | 33 | 9 | 58 | 10 | 88 | 63 |
| 1.4.3 | 3 | 0.3419 | 0.1099 | 0.0514 | 2.861 | 1.30 | 13.10 | ˜10 | 37 | 7 | 52 | 4 | 72 | 62 |
| 1.5.3 | 5 | 0.1911 | 0.08 | 0.037 | 1.031 | 0.53 | 2.56 | ˜10 | 53 | 10 | 60 | 8 | 79 | 66 |
| 1.6.2 | 5 | 0.4764 | 0.1596 | 0.0393 | 0.67 | 0.38 | 0.31 | ˜10 | 36 | 5 | 43 | 3 | 79 | 77 |
| 1.9.2 | 1 | 0.5285 | | | | 52.90 | >10 | >10 | 17 | 5 | 40 | 3 | 74 | 19 |
| 1.12.3 | 3 | 0.09892 | 0.026 | 0.0383 | 0.191 | 1.31 | 2.07 | 9.39 | 10 | 3 | 29 | 2 | 63 | 17 |
| 1.13.2 | 0 | 1.444 | 0.2911 | 0.096 | 2.971 | 3.54 | 6.82 | ˜10 | 21 | 5 | 33 | 4 | 74 | 24 |
| 2.1.2 | 9 | 0.01352 | 0.0329 | 0.0067 | 0.163 | 0.30 | 0.16 | 0.51 | 17 | 7 | 23 | 8 | 70 | 52 |
| 2.2.2 | 6 | 0.1391 | 0.0207 | 0.00076 | 0.373 | 0.50 | 1.03 | 1.66 | 44 | 7 | 65 | 12 | 79 | 60 |
| 2.4.1 | 2 | 0.9827 | 0.1119 | 0.042 | 4.685 | 3.72 | 3.12 | ˜10 | 24 | 9 | 46 | −3 | 76 | 36 |

TABLE 6
RANK ORDER

| | EC50 | EC50 | EC50 | | | | | | | | | | % Lysis |
|---|---|---|---|---|---|---|---|---|---|---|---|---|---|
| | Ramos | Ramos | Ramos | EC50 | | | | % Lysis | % Lysis | % Lysis | % Lysis | % Lysis | Daudi |
| CD20 | Apoptosis | Apoptosis | Apop- | Ramos | EC50 | EC50 | EC50 | Raji | Raji | Ramos | Ramos | Daudi | 0.001 |
| Rank | CellTiter | Alamar | tosis | Apoptosis | Raji | Ramos | Daudi | 1 ug/ml | 0.001 ug/ml | 1 ug/ml | 0.001 ug/ml | 1 ug/ml | ug/ml |
| Order | Glo | Blue | WST-1 | Annexin V | CDC | CDC | CDC | ADCC | ADCC | ADCC | ADCC | ADCC | ADCC |
| 1 | 2.1.2 | 2.2.2 | 2.2.2 | 2.1.2 | 1.3.3 | 2.1.2 | 2.1.2 | 1.1.2 | 1.1.2 | 1.1.2 | 1.1.2 | 1.1.2 | 1.1.2 |
| 2 | 1.1.2 | 1.12.3 | 1.1.2 | 1.12.3 | 2.1.2 | 1.3.3 | 1.1.2 | 1.5.3 | 1.5.3 | 1.2.1 | 2.2.2 | 1.3.3 | 1.6.2 |
| 3 | 1.12.3 | 2.1.2 | 2.1.2 | 2.2.2 | 1.6.2 | 1.1.2 | 2.2.2 | 1.2.1 | 1.3.3 | 2.2.2 | 1.3.3 | 1.2.1 | 1.5.3 |
| 4 | 2.2.2 | 1.1.2 | 1.3.3 | 1.1.2 | 1.1.2 | 1.6.2 | 1.3.3 | 2.2.2 | 2.4.1 | 1.5.3 | 1.5.3 | 1.6.2 | 1.3.3 |
| 5 | 1.5.3 | 1.3.3 | 1.5.3 | 1.6.2 | 2.2.2 | 2.2.2 | 1.12.3 | 1.4.3 | 1.2.1 | 1.3.3 | 1.2.1 | 2.2.2 | 1.4.3 |
| 6 | 1.3.3 | 1.5.3 | 1.12.3 | 1.3.3 | 1.5.3 | 1.12.3 | 1.4.3 | 1.6.2 | 2.1.2 | 1.4.3 | 2.1.2 | 1.5.3 | 2.2.2 |
| 7 | 1.4.3 | 1.4.3 | 1.6.2 | 1.5.3 | 1.4.3 | 1.5.3 | 1.5.3 | 1.3.3 | 1.4.3 | 2.4.1 | 1.13.2 | 2.4.1 | 1.2.1 |
| 8 | 1.6.2 | 2.4.1 | 2.4.1 | 1.4.3 | 1.12.3 | 2.4.1 | 1.6.2 | 2.4.1 | 2.2.2 | 1.6.2 | 1.4.3 | 1.9.2 | 2.1.2 |
| 9 | 1.9.2 | 1.6.2 | 1.4.3 | 1.13.2 | 1.13.2 | 1.13.2 | 1.13.2 | 1.13.2 | 1.6.2 | 1.9.2 | 1.9.2 | 1.13.2 | 2.4.1 |
| 10 | 2.4.1 | 1.13.2 | 1.13.2 | 2.4.1 | 2.4.1 | 1.4.3 | 2.4.1 | 1.9.2 | 1.9.2 | 1.13.2 | 1.6.2 | 1.4.3 | 1.13.2 |
| 11 | 1.13.2 | 1.2.1 | 1.2.1 | 1.2.1 | 1.9.2 | 1.2.1 | 1.2.1 | 2.1.2 | 1.13.2 | 1.12.3 | 1.12.3 | 2.1.2 | 1.9.2 |
| 12 | 1.2.1 | 1.9.2 | 1.9.2 | 1.9.2 | 1.2.1 | 1.9.2 | 1.9.2 | 1.12.3 | 1.12.3 | 2.1.2 | 2.4.1 | 1.12.3 | 1.12.3 |

### Example 11

### Whole Blood Assay

Labeling Target Cells with Calcein-AM

Tumor target cells (Ramos, Raji, Daudi) were harvested and resuspended in media at 1×10⁶ cells/ml. Calcein-AM (Sigma, cat. no. C1359) was added to final concentration of 10 μM (5 μl in 2 mL cells) and allowed to incubate for 45 minutes at 37° C. Following incubation, cells were spun at 1200 RPM for 10 minutes, supernatants discarded, and pellets resuspended in fresh growth media (2×). Pellets were resuspended to 10,000 cells/75 μl. Target cells were then plated in 75 μl (10,000 cells/well) in round bottom plates (Costar, cat. no. 3799). Antibodies were added to target cells at appropriate concentration as 50 μl/well diluted in media and allowed to incubate for 30 minutes at room temperature. Following incubation, 5011 of whole blood was added per well and allowed to incubate for 4 hours at 37° C. (Note: Whole blood was collected in tubes containing heparin.) Following incubation, plates were spun at 1200 RPM for 5 minutes. 100 ΔL supernatants were transferred to flat, black, clear bottom plates (Costar, cat. no. 3603) and fluorescence measured.

The results of cytotoxicity in whole blood assay at starting antibody concentration of 10 μg/ml shown in FIG. 13 demonstrate that the 1.1.2 and 2.1.2 antibodies mediate greater cell lysis as compared to the Rituximab control antibody, especially as demonstrated in the Raji cell line.

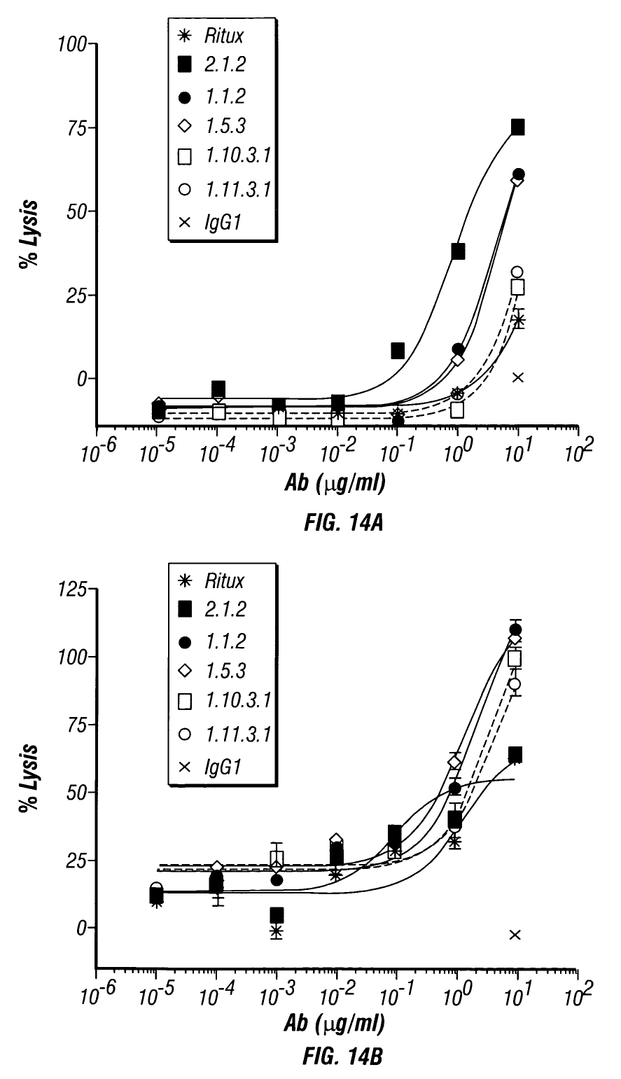

The cytotoxic activity of a panel of anti-CD20 mAbs was assessed against EHEB and Karpas-422 cell lines, using un-fractionated blood as a source of natural effectors. EHEB is a human chronic B-cell leukemia line (CLL) while Karpas-422 is a Non-Hodgkin's Lymphoma cell line. The Karpas-422 line has been previously reported to be resistant to Rituximab and complement (Br J Haematol. 2001; 114:800-9). These cell lines were evaluated in the whole blood assay in order to compare their relative sensitivity to the above antibodies and Rituximab. The data shown in FIG. 14 indicate that both EHEB and Karpas-422 cell lines are resistant to Rituximab treatment; however, anti-CD20 antibodies 2.1.2, 1.1.2, and 1.5.3 mediated significantly higher levels of cell lysis in Karpas-422 cell lines and anti-CD20 antibodies 1.1.2, 1.5.3, 1.10.3.1, and 1.11.3.1 mediated significantly higher levels of cell lysis in EHEB cell lines.

Comparison of Lytic Activity

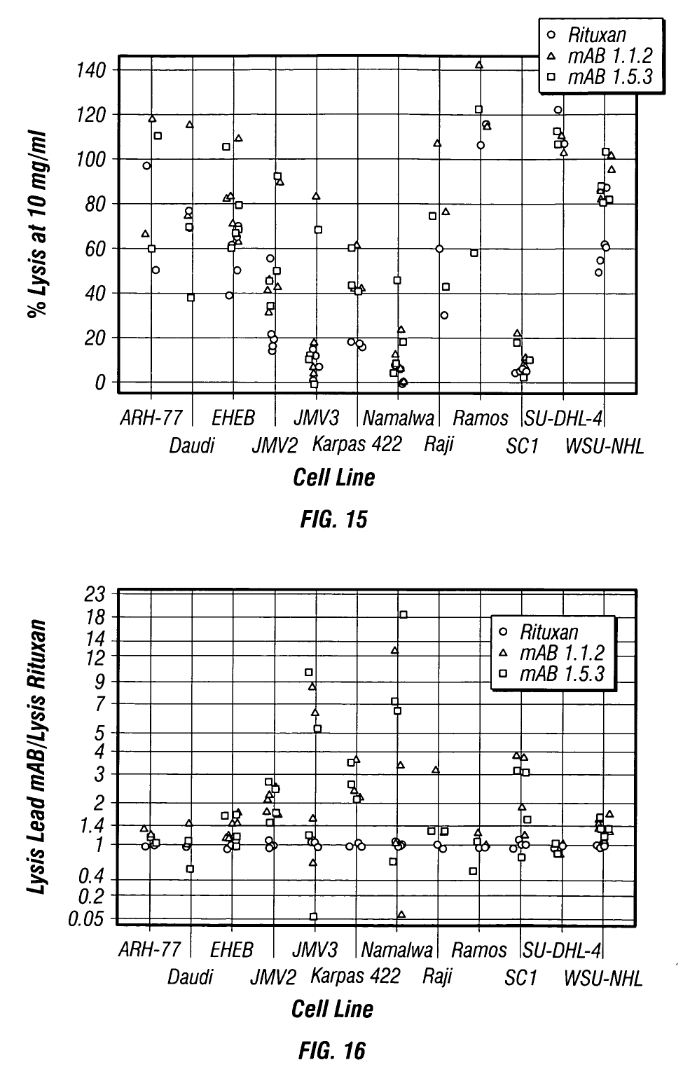

These findings were further extended to a number of non-Hodgkin lymphomas (Daudi, Ramos, ARH-77, Namalwa, Raji, SC1, WSU-NHL, SU-DHL-4 and Karpas 422) and chronic lymphocytic leukemia (EHEB, JMV-2 and JMV-3) derived cell lines. The cytolytic activity of a panel of anti-CD20 antibodies was assessed using un-fractionated blood from different human donors as variation in the activity of anti-CD20 antibodies varies as a function of a polymorphism in the FcgRIIIa receptor (Blood 2002; 99:754-758). The data shown in FIGS. 15 and 16 indicates that across donors and cell lines, the anti-CD20 antibodies 1.1.2 and 1.5.3 mediated a higher level of cell lysis at an antibody concentration of 10 μg/ml.

To further assess the cytotoxic activity of anti-CD20 antibodies, cell lines resistant to Rituximab-mediated complement mediated cytotoxicity were generated and the activity of monoclonal antibodies 1.5.3 and 1.1.2 in whole blood assay was tested. Rituximab-resistant Ramos and Raji cell lines were generated by 3 rounds of repetitive exposure to Rituximab in the presence of increasing concentration of human serum (Research Blood Components, LLC). Raji and Ramos (Burkitt's lymphoma) cells were obtained from the ATCC and were maintained in RPMI medium supplemented with 10% FBS, 1% sodium pyruvate and 1% HEPES buffer. For the first round of selection, cells were exposed to 1 μg/ml of Rituximab in presence of 20% human serum for 16 h at 37° C. and in 5% CO2. For the second and third rounds, the concentration of Rituximab was increased to 10 and 100 μg/ml, respectively in presence to up to 50% human serum. After each round, cell viability was assessed with the Guava ViaCount kit (Guava Technologies, Inc., Hayward, Calif., USA). Rituximab-resistant cells (RR-Raji or RR-Ramos) were cloned by limited dilution and clones were expanded.

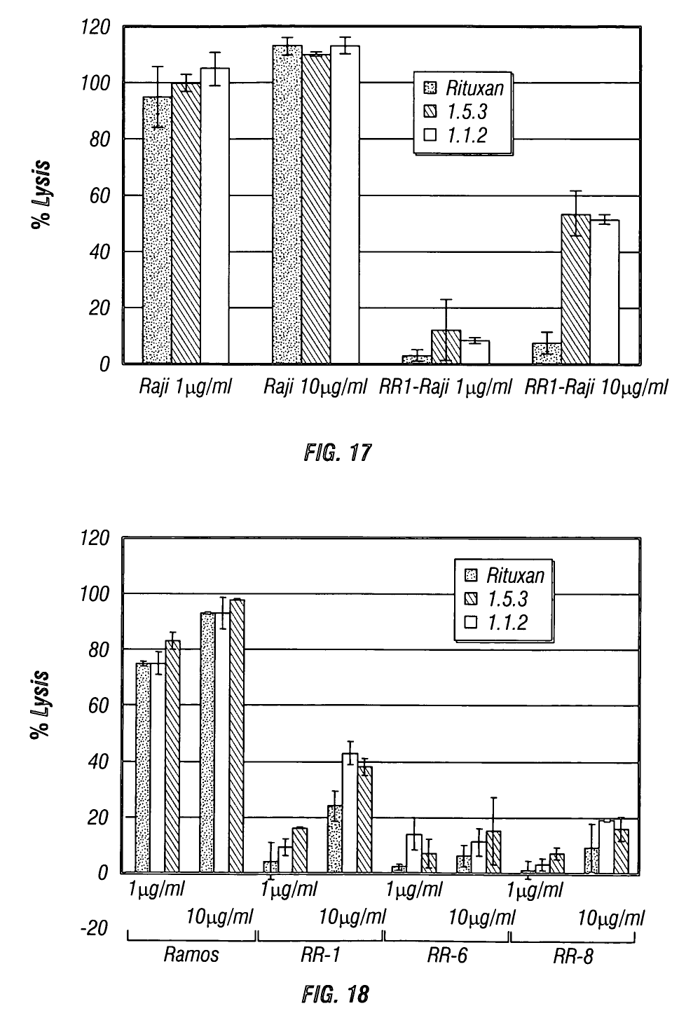

Resistance to CDC-mediated activity by Rituximab was confirmed for each clone. In the 3 clones generated for the Raji and for the Ramos cell lines, the expression of CD20 was preserved and shown to be equivalent to the parental cell lines by FACS analysis. FIG. 17 illustrates that RR1-Raji cells were not killed in the presence of 1 or 10 μg/ml of Rituximab, whereas the parental Raji cell line remained sensitive. By contrast 10 μg/ml of 1.5.3 or 1.1.2 was able to lyse about 50% of the RR1-Raji cells in this 4 h assay. Increased lytic activity was also observed in the RR1-Ramos, RR6-Ramos and RR8-Ramos cell lines with monoclonal antibodies 1.5.3 and 1.1.2 compared to Rituximab as illustrated in FIG. 18.

### Example 12

### FACS Kd Determination for Seven Purified Monoclonal Antibodies Binding to SB Cells Expressing CD20

The affinity of purified antibodies against CD20 (mAbs 1.1.2, 1.2.1, 2.1.2, 1.3.3, 1.5.3, 1.10.3.1, 1.13.2), Rituximab (positive control), and B1 was determined by FACS. Briefly, SB cells expressing CD20 were resuspended in FACS buffer (2% FBS, 0.05% NaN₃) at a concentration of approximately 5 million cells/mL. HSB cells were also resuspended in FACS buffer at a concentration of approximately 9 million cells/mL. Cells were kept on ice. Purified antibodies were serially diluted in filtered 1×PBS (2×) across 11 wells in 96-well plates. The twelfth well in each row contained buffer only. 1×PBS and cells were added to each mAb well such that the final volume was 30 μL/well and each well contained approximately 375,000 cells. The final molecular concentration ranges for the mAbs were as follows:

| mAb | Concentration |
|---|---|
| 1.1.2 = | 156-0.304 nM |
| 1.2.1 = | 202-0.098 nM |
| 2.1.2 = | 396-0.387 nM |
| 1.3.3 = | 289-0.283 nM |
| 1.5.3 = | 107-0.104 nM |
| 1.10.3.1 = | 265-0.258 nM |
| 1.13.2 = | 367-0.358 nM |
| Rituximab = | 365-0.356 nM |
| B1 = | 351-0.343 nM |

Plates were placed on a plate shaker for 3 hours at 4° C., then spun and washed 3× with PBS. 200 μL of 145 nM Cy5 goat α-human polyclonal antibody was added to each well, and 200 μL of 192 nM Cy5 goat α-mouse polyclonal antibody was added to the cells complexed with B1 antibody. Plates were then incubated for 40 minutes at 4° C., then spun and washed 3× with PBS.

The Geometric Mean Fluorescence (GMF) of 10,000 cells for each mAb concentration was determined using a FACSCalibur instrument. No significant nonspecific binding (no significant signal) was apparent from the HSB cells. In each row containing the SB cells, the signal from the twelfth well (containing buffer only) was subtracted from the signal from the first 11 wells. A nonlinear plot of GMF as a function of molecular mAb concentration was fit using Scientist software using the equation:

$$F = {P}^{′} \cdot \frac{( {K}_{D} + {L}_{T} + 1 ) - \sqrt{{( ( {K}_{D} + {L}_{T} + 1 ) )}^{2} - 4 ( {L}_{T} )}}{2}$$

In the above equation, F=Geometric mean fluorescence, L_{T}=total molecular mAb concentration, P′=proportionality constant that relates arbitrary fluorescence units to bound mAb, and K_{D}=equilibrium dissociation constant. For each mAb an estimate for K_{D} was obtained as P′ and K_{D} were allowed to float freely in the nonlinear analysis. The table below lists the resulting K_{D}s for each mAb along with the 95% confidence interval of the fit. MAbs are listed in order of decreasing affinity. Based on several previous independent measurements of Rituximab using this FACS K_{D} analysis method, the expected precision is 12% based on standard deviation (coefficient of variation) and 30% precision based on the 95% confidence intervals. However, the precision can vary with each mAb.

TABLE 7

| Sample | K_{D} (nM) | 95% CI (nM) |
|---|---|---|
| 2.1.2 | 3.8 | 0.5 |
| Rituximab | 8.1 | 1.2 |
| 1.3.3 | 8.9 | 1.1 |
| 1.5.3 | 9.1 | 1.1 |
| 1.1.2 | 11.1 | 0.5 |
| 1.10.3.1 | 11.2 | 0.8 |
| 1.2.1 | 11.9 | 1.0 |
| 1.13.2 | 23.6 | 4.4 |
| B1 | 34.1 | 2.9 |

### Example 13

### Structural Analysis of anti-CD20 Antibodies

The variable heavy chains and the variable light chains of the antibodies were sequenced to determine their DNA sequences. The complete sequence information for the anti-CD20 antibodies is provided in the sequence listing with nucleotide and amino acid sequences for each gamma and kappa chain combination. The variable heavy sequences were analyzed to determine the VH family, the D-region sequence and the J-region sequence. The sequences were then translated to determine the primary amino acid sequence and compared to the germline VH, D and J-region sequences to assess somatic hypermutations. “-” indicates identity with the germline sequence. “#” indicates an additional amino acid in the antibody sequences that is not found in the germline.

Table 8 is a table comparing the antibody heavy chain regions to their cognate germ line heavy chain region. Table 9 is a table comparing the antibody kappa light chain regions to their cognate germ line light chain region.

The variable (V) regions of immunoglobulin chains are encoded by multiple germ line DNA segments, which are joined into functional variable regions (V_{H}DJ_{H} or V_{K}J_{K}) during B-cell ontogeny. The molecular and genetic diversity of the antibody response to CD20 was studied in detail. These assays revealed several points specific to anti-CD20 antibodies.

Analysis of 36 individual antibodies specific to CD20, which were isolated from 8 hybridoma fusions, resulted in the determination that the majority of hybridoma use the same heavy and light chain pairs to form a CD20-specific paratope. The selected paratope consists of the V_{κ} A23 light chain paired with VH5-51 heavy chains. Only two mAbs were isolated in which the V_{κ} A23 light chain was paired with a VH1-18 heavy chain. A single mAb used Vλ V4-3 light chain paired with a VH6-1 heavy chain and a single mAb utilized Vλ V2-13 paired with VH1-18.

VH5 predominance was confirmed with 31 of 36 unique hybridoma-derived mAbs. The VH5-51-derived heavy chains were extensively mutated throughout CDR 1 and FR3. Sequences of VH5-51 mAbs represent multi-rearrangement events, as indicated by different patterns of substitutes and variations in CDR3 and JH usage. Identical substitutions were seen in VH5-51 heavy chains from different rearrangements (Ser³¹ to Asn³¹ in CDR1, and Met⁹³ to Ile⁹³ in FR3, for example) implying antigen-driven selection.

28 analyzed mAbs utilized V_{κ} A23-derived light chains in forming CD20-specific binding domains. All V_{κ} A23 chains were rearranged with a 9-amino-acid CDR3 and were mutated as compared to the germ line in the CDRs. In 21 mAbs, the isolated A23 kappa chains appeared to derive from a single rearrangement event, as indicated by the pattern of mutation and shared JK4 segment usage. Identical substitutions were found in different mAbs (Ser³² to Arg³² in CDR1, Ile⁵⁶ to Val⁵⁶ in CDR2, and Met⁸⁹ to Val⁸⁹ in CDR3), suggesting antigen-driven selection for these particular residues at these locations during affinity maturation.

The three lead mAbs as determined by pro-apoptotic activity as well as ability to elicit CDC and ADCC (described above), mAbs 1.1.2, 1.5.3, and 2.1.2, all utilize the Vkappa A23 light chain paired with the VH5-51 heavy chain. Both heavy and light chains were mutated, primarily in the CDRs. The lead antibodies represent a single paratope family recurring in the hybridoma.

TABLE 8
ANTI-CD-20 ANTIBODY HEAVY CHAIN SEQUENCES

| SEQ | | | | | | | |
|---|---|---|---|---|---|---|---|
| ID | | | | | | | |
| NO | ChainName | V | D | J | FR1 | CDR1 | FR2 |
| 139 | | Germline | | | QVQLVQSGAE | GYTFTSYGIS | WVRQAP |
| | | | | | VKKPGASVKV | | GQGLEW |
| | | | | | SCKAS | | MG |
| 66 | 50-001H3_3_1N1G4 | VH1- | D5- | JH4B | ---------- | ---------- | ------ |
| | | 18 | 5 | | ---------- | | ------ |
| | | | | | ----- | | -- |
| 140 | | Germline | | | QVQLVQSGAE | GYTFTSYGIS | WVRQAP |
| | | | | | VKKPGASVKV | | GQGLEW |
| | | | | | SCKAS | | MG |
| 62 | 50-001H3_2_1N1G4 | VH1- | D6- | JH4B | ---------- | --S-S----- | ------ |
| | | 18 | 13 | | ---------- | | ------ |
| | | | | | --T-- | | -- |
| 141 | | Germline | | | QVQLVESGGG | GFTFSDYYMS | WIRQAP |
| | | | | | LVKPGGSLRL | | GKGLEW |
| | | | | | SCAAS | | VS |
| 86 | 50-001H6_3_1N1G1 | VH3- | D4- | JH4B | ---------- | ---------- | ------ |
| | | 11 | 23 | | ---------- | | ------ |
| | | | | | ----- | | -- |
| 142 | | Germline | | | QVQLVESGGG | GFTFSSYGMH | WVRQAP |
| | | | | | VVQPGRSLRL | | GKGLEW |
| | | | | | SCAAS | | VA |
| 82 | 50-001H4_6_1N1G4 | VH3- | N.A. | JH4B | -------A-- | ---------- | ------ |
| | | 33 | | | ---------- | | ------ |
| | | | | | ----- | | -- |
| 143 | | Germline | | | EVQLVQSGAE | GYSFTSYWIG | WVRQMP |
| | | | | | VKKPGESLKI | | GKGLEW |
| | | | | | SCKGS | | MG |
| 10 | 50-001H1_11_3_1N1G1 | VH5-51 | N.A. | JH6B | ---------- | -----N---V | ------ |
| | | | | | ---------- | | ------ |
| | | | | | ----- | | -- |
| 18 | 50-001H1_13_1N1G1 | ″ | ″ | ″ | ---------- | -----N---- | ------ |
| | | | | | ---------- | | ------ |
| | | | | | ----- | | -- |
| 22 | 50-001H1_2_1_1N1G1 | ″ | ″ | ″ | ---------- | ---------- | ------ |
| | | | | | ---------- | | ------ |
| | | | | | ----- | | -- |
| 58 | 50-001H3_1_1N1G4 | ″ | ″ | ″ | ---------- | ---------D | ------ |
| | | | | | ---------- | | ------ |
| | | | | | ----- | | -- |
| 74 | 50-001H3_7_1N1G4 | ″ | ″ | ″ | ---------- | ---------A | ------ |
| | | | | | ---------- | | ------ |
| | | | | | ----- | | -- |
| 94 | 50-001H7_17_1N1G4 | ″ | ″ | ″ | ---------- | ----S----N | ------ |
| | | | | | ---------- | | ------ |
| | | | | | ----- | | -- |
| 98 | 50-001H7_18_1N1G4 | ″ | ″ | ″ | ---------- | -----N---N | ------ |
| | | | | | ---------- | | ------ |
| | | | | | ----- | | -- |
| 90 | 50-001H7_1_1N1G4 | ″ | ″ | ″ | ---------- | ---------- | ------ |
| | | | | | ---------- | | ------ |
| | | | | | ----- | | -- |
| 102 | 50-001H7_21_1N1G4 | ″ | ″ | ″ | ---------- | RN---N---- | ------ |
| | | | | | ---------- | | ------ |
| | | | | | ----- | | -- |
| 106 | 50-001H7_23_1_1N1G4 | ″ | ″ | ″ | G--------- | ---------- | ------ |
| | | | | | ---------- | | ------ |
| | | | | | ----- | | -- |
| 118 | 50-001H7_28_1_1N1G4 | ″ | ″ | ″ | ---------- | ---------- | ------ |
| | | | | | ---------- | | ------ |
| | | | | | ----- | | -- |
| 122 | 50-001H7_7_1N1G4 | ″ | ″ | ″ | ---------- | ---------- | ------ |
| | | | | | ---------- | | ------ |
| | | | | | ----- | | -- |
| 130 | 50-001H7_9_1N1G4 | ″ | ″ | ″ | ---------- | -----T---- | ------ |
| | | | | | ---------- | | ------ |
| | | | | | ----- | | -- |
| 144 | | Germline | | | EVQLVQSGAE | GYSFTSYWIG | WVRQMP |
| | | | | | VKKPGESLKI | | GKGLEW |
| | | | | | SCKGS | | MG |
| 110 | 50-001H7_24_1_1N1G4 | VH5-51 | D1-26 | JH3B | ---------- | -----NF--- | ------ |
| | | | | | ---S------ | | ------ |
| | | | | | ----- | | -- |
| 114 | 50-001H7_26_1N1G4 | ″ | ″ | ″ | ---------- | ------F--- | ------ |
| | | | | | ---------- | | ------ |
| | | | | | ----- | | -- |
| 145 | | Germline | | | EVQLVQSGAE | GYSFTSYWIG | WVRQMP |
| | | | | | VKKPGESLKI | | GKGLEW |
| | | | | | SCKGS | | MG |
| 126 | 50-001H7_8_1N1G4 | VH5- | D1- | JH6B | ---------- | --I------A | ------ |
| | | 51 | 26 | | ---------- | | ------ |
| | | | | | ----- | | -- |
| 146 | | Germline | | | EVQLVQSGAE | GYSFTSYWIG | WVRQMP |
| | | | | | VKKPGESLKI | | GKGLEW |
| | | | | | SCKGS | | MG |
| 38 | 50-001H1_7_1N1G1 | VH5- | D1-7 | JH6B | ---------- | ---------- | ------ |
| | | 51 | | | ---------- | | ------ |
| | | | | | ----- | | -- |
| 147 | | Germline | | | EVQLVQSGAE | GYSFTSYWIG | WVRQMP |
| | | | | | VKKPGESLKI | | GKGLEW |
| | | | | | SCKGS | | MG |
| 54 | 50-001H2_4_1N1G1 | VH5- | D3- | JH3B | ---------- | -----N---- | ------ |
| | | 51 | 10 | | ---------- | | ------ |
| | | | | | ----- | | -- |
| 148 | | Germline | | | EVQLVQSGAE | GYSFTSYWIG | WVRQMP |
| | | | | | VKKPGESLKI | | GKGLEW |
| | | | | | SCKGS | | MG |
| 14 | 50-001H1_12_1N1G1 | VH5-51 | D3-10 | JH4B | ---------- | ---------- | ----- |
| | | | | | ---------- | | S----- |
| | | | | | ----- | | --- |
| 2 | 50-001H1_1_2N1G1 | ″ | ″ | ″ | ---------- | ---------- | ------ |
| | | | | | ---------- | | ------ |
| | | | | | ----- | | -- |
| 30 | 50-001H1_5_3N1G1 | ″ | ″ | ″ | ---------- | ---------- | ------ |
| | | | | | ---------- | | ------ |
| | | | | | ----- | | -- |
| 26 | 50-001H1_4_1N1G1 | ″ | ″ | ″ | ---------- | ---------- | ------ |
| | | | | | ---------- | | ------ |
| | | | | | ----- | | -- |
| 149 | | Germline | | | EVQLVQSGAE | GYSFTSYWIG | WVRQMP |
| | | | | | VKKPGESLKI | | GKGLEW |
| | | | | | SCKGS | | MG |
| 78 | 50-001H4_2_1_1N1G4 | VH5- | D3- | JH4B | ---------- | -----N---A | ------ |
| | | 51 | 22 | | ---------- | | ------ |
| | | | | | ----- | | -- |
| 150 | | Germline | | | EVQLVQSGAE | GYSFTSYWIG | WVRQMP |
| | | | | | VKKPGESLKI | | GKGLEW |
| | | | | | SCKGS | | MG |
| 42 | 50-001H1_9_1N1G1 | VH5- | D3-3 | JH4B | ---------- | ---------- | ------ |
| | | 51 | | | ---------- | | ------ |
| | | | | | ----- | | -- |
| 151 | | Germline | | | EVQLVQSGAE | GYSFTSYWIG | WVRQMP |
| | | | | | VKKPGESLKI | | GKGLEW |
| | | | | | SCKGS | | MG |
| 6 | 50-001H1_10_3_1N1G1 | VH5-51 | D3-3 | JH6B | ---------- | ---------- | ------ |
| | | | | | ---------- | | ------ |
| | | | | | ----- | | -- |
| 46 | 50-001H2_1_1N1G1 | ″ | ″ | ″ | ---------- | ---------- | ------ |
| | | | | | ---------- | | ------ |
| | | | | | ----- | | -- |
| 50 | 50-001H2_2_1N1G1 | ″ | ″ | ″ | ---------- | ---------- | ------ |
| | | | | | ---------- | | ------ |
| | | | | | ----- | | -- |
| 152 | | Germline | | | EVQLVQSGAE | GYSFTSYWIG | WVRQMP |
| | | | | | VKKPGESLKI | | GKGLEW |
| | | | | | SCKGS | | MG |
| 34 | 50-001H1_6_1N1G1 | VH5- | D4- | JH3B | ---------- | ---------- | ------ |
| | | 51 | 11 | | ---------- | | ------ |
| | | | | | ----- | | -- |
| 153 | | Germline | | | EVQLVQSGAE | GYSFTSYWIG | WVRQMP |
| | | | | | VKKPGESLKI | | GKGLEW |
| | | | | | SCKGS | | MG |
| 70 | 50-001H3_4_1N1G4 | VH5- | D6- | JH3A | ---------- | -----N---A | ------ |
| | | 51 | 19 | | ---------- | | ------ |
| | | | | | ----- | | -- |
| 154 | | Germline | | | QVQLQQSGPG | GDSVSSNSA | WIRQSP |
| | | | | | LVKPSQTLSL | AWN | SRGLEW |
| | | | | | TCAIS | | LG |
| 134 | 50-001H8_2_1N1G4 | VH6-1 | D1- | JH4B | ---------- | -------- | ------ |
| | | | 20 | | -M-------- | VS-- | ------ |
| | | | | | ----- | | -- |

| SEQ | | | | |
|---|---|---|---|---|
| ID | | | | |
| NO | CDR2 | FR3 | CDR3 | FR4 |
| 139 | WISAYNGNTNYAQK | RVTMTTDTSTST | ##TAM#DY | WGQGT |
| | LQG | AYMELRSLRSDD | | LVTVSS |
| | | TAVYYCAR | | |
| 66 | --N----------- | --------F--- | AS---G-- | ----- |
| | -R- | -D---------- | | ------ |
| | | -------- | | |
| 140 | WISAYNGNTNYAQK | RVTMTTDTSTST | #SSW#FDY | WGQGT |
| | LQG | AYMELRSLRSDD | | LVTVSS |
| | | TAVYYCAR | | |
| 62 | -------H-R---- | -----S------ | A---Y--C | ----- |
| | --- | ------------ | | ------ |
| | | -------- | | |
| 141 | YISSSGSTIYYADS | RFTISRDNAKNS | ###YGGN# | WGQGT |
| | VKG | LYLQMNSLRAED | #YFDY | LVTVSS |
| | | TAVYYCAR | | |
| 86 | ------T------- | ------------ | DLY---- | ----- |
| | --- | ------------ | SY---- | ------ |
| | | -------- | | |
| 142 | VIWYDGSNKYYADS | RFTISRDNSKNT | ###FDY | WGQGT |
| | VKG | LYLQMNSLRAED | | LVTVSS |
| | | TAVYYCA# | | |
| 82 | ---H---K---E-- | ------------ | NWF--- | ----- |
| | --- | ------------ | | ------ |
| | | -------R | | |
| 143 | IIYPGDSDTRYSPS | QVTISADKSIST | ###YYYYY | WGQGT |
| | FQG | AYLQWSSLKASD | GMDV | TVTVSS |
| | | TAMYYCAR | | |
| 10 | -------------- | ------------ | HGD----- | ----- |
| | --- | ------------ | ---- | ------ |
| | | -------- | | |
| 18 | -------------- | ------------ | QGD--H- | ----- |
| | --- | ------------ | S---- | ------ |
| | | -------- | | |
| 22 | -------------- | ------------ | LGD--N-- | ----- |
| | --- | ------------ | ---- | ------ |
| | | -------- | | |
| 58 | S------------- | ------------ | QGASG--- | ----- |
| | --- | ------------ | ---- | ------ |
| | | -------- | | |
| 74 | -------------- | ------------ | TGS--N- | ----- |
| | --- | ------N----- | C---- | ------ |
| | | -------- | | |
| 94 | -------------- | ------------ | VGD--S-- | ----- |
| | --- | -----R------ | ---- | ------ |
| | | --I----- | | |
| 98 | -------------- | ----------N- | QGGH--- | ----- |
| | --- | ------------ | S---- | ------ |
| | | -------- | | |
| 90 | -------------- | ----------R- | IGDH-H- | ----- |
| | --- | ------------ | N---- | ------ |
| | | -------- | | |
| 102 | -------------- | ------------ | TGS-S--- | ----- |
| | --- | ------------ | ---- | ------ |
| | | -------- | | |
| 106 | -------------- | --I--------- | TGD--S- | ----- |
| | --- | ------------ | H---- | ------ |
| | | -------- | | |
| 118 | -------------- | ------------ | TGD-HN-- | ----- |
| | --- | ------------ | ---- | ------ |
| | | -------- | | |
| 122 | ----A--------- | ---------LN- | IGDF--- | ----- |
| | --- | -----R------ | S---- | ------ |
| | | -------- | | |
| 130 | -------------- | ------------ | IGD--S- | ----- |
| | --- | ------------ | S-L-- | ------ |
| | | -------- | | |
| 144 | IIYPGDSDTRYSPS | QVTISADKSIST | ###YSGSY | WGQGT |
| | FQG | AYLQWSSLKASD | Y##AFDI | MVTVSS |
| | | TAMYYCAR | | |
| 110 | -------------- | ------------ | HPP----- | ----- |
| | --- | T----------- | -AD---- | ------ |
| | | -------- | | |
| 114 | -------------- | L----------- | HPP----- | ----- |
| | -R- | ---H-------- | -AD---- | ------ |
| | | --IF---- | | |
| 145 | IIYPGDSDTRYSPS | QVTISADKSIST | VGATNYYY | WGQGT |
| | FQG | AYLQWSSLKASD | GMDV | TVTVSS |
| | | TAMYYCAR | | |
| 126 | -------------- | -I---------- | --T----- | ----- |
| | --- | ------------ | ---- | S----- |
| | | -------- | | |
| 146 | IIYPGDSDTRYSPS | QVTISADKSIST | TGT##YYY | WGQGT |
| | FQG | AYLQWSSLKASD | GMDV | TVTVSS |
| | | TAMYYCAR | | |
| 38 | -------------- | ------------ | ---TD-- | ----- |
| | --- | ------------ | S---- | ------ |
| | | -------- | | |
| 147 | IIYPGDSDTRYSPS | QVTISADKSIST | ###YYGSG | WGQGT |
| | FQG | AYLQWSSLKASD | S#AFDI | MVTVSS |
| | | TAMYYCAR | | |
| 54 | -------------- | ------------ | HGD--A- | ----- |
| | --- | ------------ | E-S---- | ------ |
| | | -------- | | |
| 148 | IIYPGDSDTRYSPS | QVTISADKSIST | ###YGSGS | WGQGT |
| | FQG | AYLQWSSLKASD | ##FDY | LVTVSS |
| | | TAMYYCAR | | |
| 14 | -------------- | ------------ | HPS----- | ----- |
| | --- | ------------ | PN--- | ------ |
| | | --I----- | | |
| 2 | -----------R-- | ----------T- | HPS----- | ----- |
| | --- | ------------ | PN--- | ------ |
| | | -------- | | |
| 30 | -------------- | ----------T- | HPS----- | ----- |
| | --- | ------------ | PN--- | ------ |
| | | -------- | | |
| 26 | -------------- | ----------T- | HPS----- | ----- |
| | --- | ------------ | PN--- | ------ |
| | | -------- | | |
| 149 | IIYPGDSDTRYSPS | QVTISADKSIST | ###YYDSS | WGQGT |
| | FQG | AYLQWSSLKASD | ##DY | LVTVSS |
| | | TAMYYCAR | | |
| 78 | -------------- | ------------ | QGD----- | ----- |
| | --- | ------------ | GP-- | ------ |
| | | ------T- | | |
| 150 | IIYPGDSDTRYSPS | QVTISADKSIST | ##DFWSGY | WGQGT |
| | FQG | AYLQWSSLKASD | Y#FDY | LVTVSS |
| | | TAMYYCAR | | |
| 42 | -------------- | ------------ | HG------ | ----- |
| | --- | ------------ | -S--- | ------ |
| | | -------- | | |
| 151 | IIYPGDSDTRYSPS | QVTISADKSIST | ##DFWSGY | WGQGT |
| | FQG | AYLQWSSLKASD | YT#GMDV | TVTVSS |
| | | TAMYYCAR | | |
| 6 | -------------- | ------------ | MG------ | ----- |
| | --- | ------------ | --R---- | ------ |
| | | -------- | | |
| 46 | -------------- | ------------ | QG------ | ----- |
| | --- | ------------ | NGGMDV | ------ |
| | | -------- | | |
| 50 | ---------K---- | ------------ | QG----- | ----- |
| | --- | ------------ | NGGMDV | ------ |
| | | -------- | | |
| 152 | IIYPGDSDTRYSPS | QVTISADKSIST | ##DYSN## | WGQGT |
| | FQG | AYLQWSSLKASD | AFDI | MVTVSS |
| | | TAMYYCAR | | |
| 34 | -------------- | ------------ | HG---- | ----- |
| | --- | ------------ | ID---- | ------ |
| | | -------- | | |
| 153 | IIYPGDSDTRYSPS | QVTISADKSIST | ###YSSG# | WGQGT |
| | FQG | AYLQWSSLKASD | ##AFDV | MVTVSS |
| | | TAMYYCAR | | |
| 70 | --F---------- | ------------ | HRD-T-- | ----- |
| | A--- | ---H-------- | GPD---- | ------ |
| | | -------- | | |
| 154 | RTYYRSKWYNDYAV | RITINPDTSKNQ | ###WN##F | WGQGT |
| | SVKS | FSLQLNSVTPED | DY | LVTVSS |
| | | TAVYYCAR | | |
| 134 | -----F--F----- | ------------ | IDI--DV- | ----- |
| | ---- | ---R-------- | -- | ------ |
| | | --L----- | | |

TABLE 9
ANTI-CD20 ANTIBODY LIGHT CHAIN SEQUENCES

| SEQ | | | | | | |
|---|---|---|---|---|---|---|
| ID | Chain | | | | | |
| NO | Name | V | J | FR1 | CDR1 | FR2 |
| 155 | | Germline | | DVVMTQSP | RSSQSLVYSDGNTYLN | WFQQRPGQSP |
| | | | | LSLPVTLG | | RRLIY |
| | | | | QPASISC | | |
| 36 | 50-001H1_6_1N1K | A1 | JK3 | -------- | ------------A--- | ---------- |
| | | | | -------- | | ----- |
| | | | | ------- | | |
| 156 | | Germline | | DVVMTQSP | RSSQSLVYSDGNTYLN | WFQQRPGQSP |
| | | | | LSLPVTLG | | RRLIY |
| | | | | QPASISC | | |
| 20 | 50-001H1_13_1N1K | A1 | JK4 | -------- | --------N--D---- | ---------- |
| | | | | ---S---- | | -L--- |
| | | | | ------- | | |
| 124 | 50-001H7_7_1N1K | ″ | ″ | -------- | --R--------S---- | ---------- |
| | | | | -------- | | ----- |
| | | | | ------- | | |
| 157 | | Germline | | DVVMTQSP | RSSQSLVYSDGNTYLN | WFQQRPGQSP |
| | | | | LSLPVTLG | | RRLIY |
| | | | | QPASISC | | |
| 44 | 50-001H1_9_1N1K | A1 | JK5 | -------- | ---------------- | ---------- |
| | | | | -------- | | ----- |
| | | | | ------- | | |
| 158 | | Germline | | DIVMTQTP | RSSQSLVHSDGNTYLS | WLQQRPGQPP |
| | | | | LSSPVTLG | | RLLIY |
| | | | | QPASISC | | |
| 12 | 50-001H1_11_3_1N1K | A23 | JK4 | -------- | ----R------H---- | ---------- |
| | | | | -------- | | ----S |
| | | | | ------- | | |
| 4 | 50-001H1_1_2N1K | ″ | ″ | -------- | -------Y-------- | ---------- |
| | | | | -------- | | ----- |
| | | | | ------- | | |
| 16 | 50-001H1_12_1N1K | ″ | ″ | -------- | -------Y-------- | ---------- |
| | | | | -------- | | ---F- |
| | | | | ------- | | |
| 32 | 50-001H1_5_3N1K | ″ | ″ | -------- | -------Y-------- | ---------- |
| | | | | -------- | | ----- |
| | | | | ------- | | |
| 40 | 50-001H1_7_1N5K | ″ | ″ | -------- | -------------F-- | ---------- |
| | | | | -------- | | ----- |
| | | | | ------- | | |
| 56 | 50-001H2_4_1N1K | ″ | ″ | -------- | -------------F-- | ---------- |
| | | | | -------- | | ----- |
| | | | | ------- | | |
| 60 | 50-001H3_1_1N1K | ″ | ″ | ------S- | -Y------R------- | ---------- |
| | | | | -------- | | --V-H |
| | | | | ------- | | |
| 64 | 50-001H3_2_1N1K | ″ | ″ | -------- | ---------------- | ---------- |
| | | | | -------- | | ----- |
| | | | | ------- | | |
| 68 | 50-001H3_3_1N1K | ″ | ″ | E------- | K--------------- | ---------- |
| | | | | -------- | | ----- |
| | | | | ------- | | |
| 72 | 50-001H3_4_1N1K | ″ | ″ | -------- | -------Y-------- | --H------- |
| | | | | -------- | | ----- |
| | | | | ------- | | |
| 76 | 50-001H3_7_1N1K | ″ | ″ | -------- | ---------------- | ---------- |
| | | | | -------- | | ----- |
| | | | | ------- | | |
| 80 | 50-001H4_2_1_1N1K | ″ | ″ | -------- | -------YR------- | ---------- |
| | | | | -------- | | ----- |
| | | | | ------- | | |
| 84 | 50-001H4_6_1N1K | ″ | ″ | K------- | ---------------- | -F-------- |
| | | | | -------- | | ----N |
| | | | | ------- | | |
| 96 | 50-001H7_17_1N1K | ″ | ″ | -------- | -----------K---- | ---------- |
| | | | | P-----R- | | ----- |
| | | | | ------- | | |
| 100 | 50-001H7_18_1N1K | ″ | ″ | -------- | --R---L--------- | ---------- |
| | | | | -------- | | ----- |
| | | | | ------- | | |
| 92 | 50-001H7_1_1N1K | ″ | ″ | -------- | ---------------- | ---------- |
| | | | | -------- | | ---L- |
| | | | | ------- | | |
| 108 | 50-001H | ″ | ″ | -------- | -------------F-- | ---------- |
| | 7_23_1_1N1K | | | -------- | | ----- |
| | | | | ------- | | |
| 112 | 50-001H | ″ | ″ | ---L---- | -----------H---- | ---------- |
| | 7_24_1_1N1K | | | -------- | | ----- |
| | | | | ------- | | |
| 116 | 50-001H | ″ | ″ | -------- | -------S-------- | ---------- |
| | 7_26_1N1K | | | -------- | | ----- |
| | | | | ------- | | |
| 120 | 50-001H | ″ | ″ | ------S- | -------------F-- | ---------- |
| | 7_28_1_1N1K | | | -------- | | ----- |
| | | | | ------- | | |
| 128 | 50-001H | ″ | ″ | -------- | -------------F-- | ---------- |
| | 7_8_1N1K | | | -------- | | ----- |
| | | | | ------- | | |
| 132 | 50-001H | ″ | ″ | -------- | -----------H---- | ---------- |
| | 7_9_1N1K | | | -------- | | ---F- |
| | | | | ------- | | |
| 159 | | Germline | | DIVMTQTP | RSSQSLVHSDGNTYLS | WLQQRPGQPP |
| | | | | LSSPVTLG | | RLLIY |
| | | | | QPASISC | | |
| 24 | 50-001H | A23 | JK5 | -------- | -----------K---- | ---------- |
| | 1_2_1_1N1K | | | -------- | | ----- |
| | | | | ------- | | |
| 48 | 50-001H | ″ | ″ | -------- | -------SR------- | ---------- |
| | 2_1_1N1K | | | H------- | | ----- |
| | | | | ------- | | |
| 52 | 50-001H | ″ | ″ | -------- | -------SR------- | ---------- |
| | 2_2_1N1K | | | -------- | | ----- |
| | | | | ------- | | |
| 104 | 50-001H | ″ | ″ | -------- | ----------R----- | ---------- |
| | 7_21_1N1K | | | -------- | | ----- |
| | | | | ------- | | |
| 28 | 50-001H | ″ | ″ | -------- | ---------------- | ---------- |
| | 1_4_1N1K | | | -------- | | ----- |
| | | | | ------- | | |
| 160 | | Germline | | EIVMTQSP | RASQSVSSNLA | WYQQKPGQAP |
| | | | | ATLSVSPG | | RLLIY |
| | | | | ERATLSC | | |
| 8 | 50-001H | L2 | JK4 | -------- | ----------- | ---------- |
| | 1_10_3_1N1K | | | -------- | | ----S |
| | | | | ------- | | |
| 161 | | Germline | | SSELTQDP | QGDSLRSYYAS | WYQQKPGQAP |
| | | | | AVSVALGQ | | VLVIY |
| | | | | TVRITC | | |
| 88 | 50-001H | V2- | JL2 | -------- | ----------- | ---------- |
| | 6_3_1N1L | 13 | 2 | -------- | | ----- |
| | | | | ------ | | |
| 162 | | Germline | | QPVLTQPT | TLRSGINLGSYRIF | WYQQKPESPP |
| | | | | SLSASPGA | | RYLLS |
| | | | | SARLTC | | |
| 136 | 50-001H | V4-3 | JL2 | -------- | -------------- | ---------- |
| | 8_2_1N1L | | | -------- | | ----- |
| | | | | ------ | | |

| SEQ | | | | |
|---|---|---|---|---|
| ID | | | | |
| NO | CDR2 | FR3 | CDR3 | FR4 |
| 155 | KVSNWDS | GVPDRFSGSGS | MQGTH## | FGPGTK |
| | | GTDFTLKISRV | | VDIK |
| | | EAEDVGVYYC | | |
| 36 | ------- | ----------- | ---I-CT | ------ |
| | | --------R-- | | -H-- |
| | | ------A--- | | |
| 156 | KVSNWDS | GVPDRFSGSGS | MQGTHWPLT | FGGGTK |
| | | GTDFTLKISRV | | VEIK |
| | | EAEDVGVYYC | | |
| 20 | ------- | ----------- | --------- | ------ |
| | | -------V--- | | ---- |
| | | ---------- | | |
| 124 | ------- | -------A--- | --------LA | ------ |
| | | ----------- | | ---- |
| | | ----D---H- | | |
| 157 | KVSNWDS | GVPDRFSGSGS | MQGTHWP#IT | FGQGTR |
| | | GTDFTLKISRV | | LEIK |
| | | EAEDVGVYYC | | |
| 44 | ------- | ----------- | -------S-- | ------ |
| | | ----------- | | ---- |
| | | ---------- | | |
| 158 | KISNRFS | GVPDRFSGSGA | MQATQF##T | FGGGTK |
| | | GTDFTLKISRV | | VEIK |
| | | EAEDVGVYYC | | |
| 12 | -V----- | ----------- | ------PL- | ------ |
| | | ----------- | | ---- |
| | | ------I--- | | |
| 4 | ------- | ----------- | V-----PL- | ------ |
| | | ----------- | | ---- |
| | | ---------- | | |
| 16 | ------- | ----------- | V-----PL- | ------ |
| | | ----------- | | ---- |
| | | ---------- | | |
| 32 | ------- | ----------- | V-----PL- | ------ |
| | | ----------- | | ---- |
| | | ---------- | | |
| 40 | ------- | ----------- | --L---PL- | ------ |
| | | ----------- | | ---R |
| | | ---------- | | |
| 56 | ------- | ----------- | --G---PL- | ------ |
| | | ----------- | | ---- |
| | | ---------- | | |
| 60 | ------- | ----------- | --T---PL- | ------ |
| | | ----------- | | ---- |
| | | ------F--- | | |
| 64 | ------- | ----------- | ------PL- | ------ |
| | | ----------- | | ---- |
| | | ---------- | | |
| 68 | ------- | ----------- | --T-Y-PL- | ------ |
| | | ----------- | | ---- |
| | | ---------- | | |
| 72 | ------- | ----------- | --T---PL- | ------ |
| | | ----------- | | -K-- |
| | | ------L--- | | |
| 76 | -L----- | -I--------- | ------PL- | ------ |
| | | ----------- | | ---- |
| | | ---------- | | |
| 80 | ------- | ----------- | ------PL- | ------ |
| | | ----------- | | ---- |
| | | ---------- | | |
| 84 | ------- | ----------- | ------PL- | ------ |
| | | ----------- | | ---- |
| | | ---------- | | |
| 96 | ------- | ---G------- | ------PL- | ------ |
| | | ----------- | | ---- |
| | | ---------- | | |
| 100 | -L---V- | ----------- | --S---PL- | ------ |
| | | ----------- | | -K-- |
| | | ---------- | | |
| 92 | -N----- | ----------- | ------PL- | ------ |
| | | ----------- | | ---- |
| | | ---------- | | |
| 108 | ------- | ----------- | ------PL- | ------ |
| | | ----------- | | ---- |
| | | ------I--- | | |
| 112 | ------- | ----------- | ------PL- | ------ |
| | | ----------- | | ---- |
| | | G--------- | | |
| 116 | ----L-- | ----------- | ------PL- | ------ |
| | | ----------- | | ---- |
| | | ------L--- | | |
| 120 | ------- | ----------- | I-----PL- | ------ |
| | | ----------- | | ---- |
| | | ---------- | | |
| 128 | ------- | ----------- | ------PL- | ------ |
| | | ----------- | | ---- |
| | | ---------- | | |
| 132 | ------- | ----------- | ------PL- | ------ |
| | | ----------- | | -D-- |
| | | ------I--- | | |
| 159 | KISNRFS | GVPDRFSGSGA | MQATQFPIT | FGQGTR |
| | | GTDFTLKISRV | | LEIK |
| | | EAEDVGVYYC | | |
| 24 | -S----- | ----------- | V-------- | ------ |
| | | ----------- | | ---- |
| | | ---------- | | |
| 48 | ------- | ---N------- | -------L- | ------ |
| | | ----------- | | ---- |
| | | K--------- | | |
| 52 | ------- | ----------- | --------- | ------ |
| | | ----------- | | ---- |
| | | -V-------- | | |
| 104 | -V----- | ---E------ | V-E-L---- | I----- |
| | | T---------- | | ---- |
| | | ----------- | | |
| 28 | ------- | ----------- | ---I----- | ------ |
| | | ----------- | | ---- |
| | | ---------- | | |
| 160 | GASTRAT | GIPARFSGSGS | QQYNNW##T | FGGGTK |
| | | GTEFTLTISSL | | VEIK |
| | | QSEDFAVYYC | | |
| 8 | ------- | ----------- | H---D-SL- | ------ |
| | | ----------- | | ---- |
| | | ---------- | | |
| 161 | GKNNRPS | GIPDRFSGSSS | NSRDSSGNH#V | FGGGTK |
| | | GNTASLTITGA | | LTVL |
| | | QAEDEADYYC | | |
| 88 | ------- | ---A-----D- | ---------V- | ------ |
| | | ----------- | | ---- |
| | | ---------- | | |
| 162 | YYSDSSK | HQGSGVPSRFS | EADYYCMIWHSSA#V | FGGGTK |
| | | GSKDASSNAGI | | LTVL |
| | | LVISGLQSED | | |
| 136 | -----R- | ----------- | ------IF----- | ------ |
| | | ----------- | W- | ---- |
| | | ---------- | | |

### Example 14

### Determination of Canonical Class Antibodies

Chothia, et al. have described antibody structure in terms of “canonical classes” for the hypervariable regions of each immunoglobulin chain (J Mol. Biol. 1987 Aug. 20; 196(4):901-17). The atomic structures of the Fab and VL fragments of a variety of immunoglobulins were analyzed to determine the relationship between their amino acid sequences and the three-dimensional structures of their antigen binding sites. Chothia, et al. found that there were relatively few residues that, through their packing, hydrogen bonding or the ability to assume unusual phi, psi or omega conformations, were primarily responsible for the main-chain conformations of the hypervariable regions. These residues were found to occur at sites within the hypervariable regions and in the conserved β-sheet framework. By examining sequences of immunoglobulins having unknown structure, Chothia, et al. show that many immunogloblins have hypervariable regions that are similar in size to one of the known structures and additionally contained identical residues at the sites responsible for the observed conformation.

Their discovery implied that these hypervariable regions have conformations close to those in the known structures. For five of the hypervariable regions, the repertoire of conformations appeared to be limited to a relatively small number of discrete structural classes. These commonly occurring main-chain conformations of the hypervariable regions were termed “canonical structures.” Further work by Chothia, et al. (Nature 1989 Dec. 21-28; 342(6252):877-83) and others (Martin, et al. J Mol. Biol. 1996 Nov. 15; 263(5):800-15) confirmed that there is a small repertoire of main-chain conformations for at least five of the six hypervariable regions of antibodies.

The CDRs of each antibody described above were analyzed to determine their canonical class. As is known, canonical classes have only been assigned for CDR1 and CDR2 of the antibody heavy chain, along with CDR1, CDR2 and CDR3 of the antibody light chain. The tables below summarize the results of the analysis. The Canonical Class data is in the form of HCDR1-HCDR2-LCDR1-LCDR2-LCDR3 (H1-H2-L1-L2-L3), wherein “HCDR” refers to the heavy chain CDR and “LCDR” refers to the light chain CDR. Thus, for example, a canonical class of 1-3-2-1-5 refers to an antibody that has a HCDR1 that falls into canonical class 1, a HCDR2 that falls into canonical class 3, a LCDR1 that falls into canonical class 2, a LCDR2 that falls into canonical class 1, and a LCDR3 that falls into canonical class 5.

Assignments were made to a particular canonical class where the antibody sequence matched the key amino acids defined for each canonical class. The amino acids defined for each antibody can be found, for example, in the articles by Chothia, et al. referred to above. Table 10 and Table 11 report the canonical class data for each of the CD20 antibodies. Where there was no matching canonical class with the same CDR length, the canonical class assignment is marked with a letter s and a number, such as “s18”, meaning the CDR is of size 18. Where the structure of the CDR is predicted to be similar to a defined canonical class but with a likely deviation, the canonical class assignment is marked with a ‘ˆ’. Where there was no sequence data available for one of the heavy or light chains, the canonical class is marked with “Z”.

TABLE 10

| Antibody (sorted) | H1-H2-L1-L2-L3 | H3length |
|---|---|---|
| 50-001H1_10_3_1 | 1-2-2-1-s9 | 15 |
| 50-001H1_11_3_1 | 1-2-4-1-1 | 12 |
| 50-001H1_12_1 | 1-2-4-1-1 | 13 |
| 50-001H1_13_1 | 1-2-4-1-1 | 12 |
| 50-001H1_1_1 | 1-2-8-1-1 | 13 |
| 50-001H1_2_1_1 | 1-2-4-1-1 | 12 |
| 50-001H1_3_1 | 1-2-4-1-1 | 13 |
| 50-001H1_4_1 | 1-2-8-1-1 | 13 |
| 50-001H1_5_1 | 1-2-4-1-1 | 13 |
| 50-001H1_6_1 | 1-2-4-1-4 | 12 |
| 50-001H1_7_1 | 1-2-4-1-1 | 12 |
| 50-001H1_9_1 | 1-2-4-1-s10 | 13 |
| 50-001H2_1_1 | 1-2-4-1-1 | 13 |
| 50-001H2_2_1 | 1-2-4-1-1 | 13 |
| 50-001H2_4_1 | 1-2-4-1-1 | 14 |
| 50-001H3_1_1 | 1-2-4-1-1 | 12 |
| 50-001H3_2_1 | 1-2-4-1-1 | 8 |
| 50-001H3_3_1 | 1-2-4-1-1 | 8 |
| 50-001H3_4_1 | 1-2-4-1-1 | 14 |
| 50-001H3_7_1 | 1-2-4-1-1 | 12 |
| 50-001H4_2_1_1 | 1-2-4-1-1 | 12 |
| 50-001H4_5_1 | 1-2-Z-Z-Z | 12 |
| 50-001H4_6_1 | 1-3-4-1-1 | 6 |
| 50-001H6_3_1 | 1-3-9-1-5 | 13 |
| 50-001H7_17_1 | 1-2-4-1-1 | 12 |
| 50-001H7_18_1 | 1-2-4-1-1 | 12 |
| 50-001H7_1_1 | 1-2-4-1-1 | 12 |
| 50-001H7_21_1 | 1-2-4\hat{ }-1-1 | 12 |
| 50-001H7_23_1_1 | 1-2-4-1-1 | 12 |
| 50-001H7_24_1_1 | 1-2-4-1-1 | 15 |
| 50-001H7_26_1 | 1-2-4-1-1 | 15 |
| 50-001H7_28_1_1 | 1-2-4-1-1 | 12 |
| 50-001H7_7_1 | 1-2-4-1-s10 | 12 |
| 50-001H7_8_1 | 1-2-4-1-1 | 12 |
| 50-001H7_9_1 | 1-2-4-1-1 | 12 |

TABLE 11

| Antibody | H1-H2-L1-L2-L3 (sorted) | H3length |
|---|---|---|
| 50-001H1_10_3_1 | 1-2-2-1-s9 | 15 |
| 50-001H7_21_1 | 1-2-4\hat{ }-1-1 | 12 |
| 50-001H3_2_1 | 1-2-4-1-1 | 8 |
| 50-001H3_3_1 | 1-2-4-1-1 | 8 |
| 50-001H1_11_3_1 | 1-2-4-1-1 | 12 |
| 50-001H1_13_1 | 1-2-4-1-1 | 12 |
| 50-001H1_2_1_1 | 1-2-4-1-1 | 12 |
| 50-001H1_7_1 | 1-2-4-1-1 | 12 |
| 50-001H3_1_1 | 1-2-4-1-1 | 12 |
| 50-001H3_7_1 | 1-2-4-1-1 | 12 |
| 50-001H4_2_1_1 | 1-2-4-1-1 | 12 |
| 50-001H7_17_1 | 1-2-4-1-1 | 12 |
| 50-001H7_18_1 | 1-2-4-1-1 | 12 |
| 50-001H7_1_1 | 1-2-4-1-1 | 12 |
| 50-001H7_23_1_1 | 1-2-4-1-1 | 12 |
| 50-001H7_28_1_1 | 1-2-4-1-1 | 12 |
| 50-001H7_8_1 | 1-2-4-1-1 | 12 |
| 50-001H7_9_1 | 1-2-4-1-1 | 12 |
| 50-001H1_12_1 | 1-2-4-1-1 | 13 |
| 50-001H1_3_1 | 1-2-4-1-1 | 13 |
| 50-001H1_5_1 | 1-2-4-1-1 | 13 |
| 50-001H2_1_1 | 1-2-4-1-1 | 13 |
| 50-001H2_2_1 | 1-2-4-1-1 | 13 |
| 50-001H2_4_1 | 1-2-4-1-1 | 14 |
| 50-001H3_4_1 | 1-2-4-1-1 | 14 |
| 50-001H7_24_1_1 | 1-2-4-1-1 | 15 |
| 50-001H7_26_1 | 1-2-4-1-1 | 15 |
| 50-001H1_6_1 | 1-2-4-1-4 | 12 |
| 50-001H7_7_1 | 1-2-4-1-s10 | 12 |
| 50-001H1_9_1 | 1-2-4-1-s10 | 13 |
| 50-001H1_1_1 | 1-2-8-1-1 | 13 |
| 50-001H1_4_1 | 1-2-8-1-1 | 13 |
| 50-001H4_5_1 | 1-2-Z-Z-Z | 12 |
| 50-001H4_6_1 | 1-3-4-1-1 | 6 |
| 50-001H6_3_1 | 1-3-9-1-5 | 13 |

Table 12 is an analysis of the number of antibodies per class. The number of antibodies having the particular canonical class designated in the left column is shown in the right column. The one mAb lacking one chain sequence data and thus having “Z” in the canonical assignment is not included in this counting. The most commonly seen structure was 1-2-4-1-1, with 25 out of 34 mAbs having both heavy and light chain sequences of this combination.

TABLE 12
NUMBER OF CD20 ANTIBODIES IN EACH CANONICAL CLASS
COMBINATION

| H1-H2-L1-L2-L3 | Count |
|---|---|
| 1-2-2-1-s9 | 1 |
| 1-2-4\hat{ }-1-1 | 1 |
| 1-2-4-1-1 | 25 |
| 1-2-4-1-4 | 1 |
| 1-2-4-1-s10 | 2 |
| 1-2-8-1-1 | 2 |
| 1-3-4-1-1 | 1 |
| 1-3-9-1-5 | 1 |

### Example 15

### Epitope Characterization: Spot-Synthesis of Synthetic Peptides

Detection of Low Affinities Peptide-Antibody Interation

An overlapping peptide scan spanning the 43 amino acid extracellular domain of the human CD20 sequence was prepared by automated SPOT synthesis. A series of 33 12-mer peptides was synthesized as spots on polypropylene membrane sheets. The peptide array spanned amino acid residues 142-185 of the CD20 sequence. Each consecutive peptide was offset by one reside from the previous peptide, yielding a nested, overlapping library of arrayed oligopeptides as shown in Table 13.

TABLE 13

| Peptide | SEQ ID NO. |
|---|---|
| 1 KISHFLKMESLN | 163 |
| 2 ISHFLKMESLNF | 164 |
| 3 SHFLKMESLNFI | 165 |
| 4 HFLKMESLNFIR | 166 |
| 5 FLKMESLNFIRA | 167 |
| 6 LKMESLNFIRAH | 168 |
| 7 KMESLNFIRAHT | 169 |
| 8 MESLNFIRAHTP | 170 |
| 9 ESLNFIRAHTPY | 171 |
| 10 SLNFIRAHTPYI | 172 |
| 11 LNFIRAHTPYIN | 173 |
| 12 NFIRAHTPYINI | 174 |
| 13 FIRAHTPYINIY | 175 |
| 14 IRAHTPYINIYN | 176 |
| 15 RAHTPYINIYNC | 177 |
| 16 AHTPYINIYNCE | 178 |
| 17 HTPYINIYNCEP | 179 |
| 18 TPYINIYNCEPA | 180 |
| 18 TPYINIYNCEPA | 181 |
| 19 PYINIYNCEPAN | 182 |
| 20 YINIYNCEPANP | 183 |
| 21 INIYNCEPANPS | 184 |
| 22 NIYNCEPANPSE | 185 |
| 23 IYNCEPANPSEK | 186 |
| 24 YNCEPANPSEKN | 187 |
| 25 NCEPANPSEKNS | 188 |
| 26 CEPANPSEKNSP | 189 |
| 27 EPANPSEKNSPS | 190 |
| 28 PANPSEKNSPST | 191 |
| 29 ANPSEKNSPSTQ | 192 |
| 30 NPSEKNSPSTQY | 193 |
| 32 SEKNSPSTQYCY | 194 |
| 33 EKNSPSTQYCYS | 195 |

Briefly, peptide scans containing 12-mer CD20 derived peptides were probed with monoclonal antibodies. The pattern of the peptide bound antibody to CD20 derived peptides was immobilized by electrochemical transfer of the antibody-peptide complex onto PVDF membranes. Electrotransfer was carried out in a fractionated manner onto three separate PVDF membranes (high to low background). The monoclonal antibodies were detected with a peroxidase-labeled Goat-anti-human IgG antibody and chemiluminescense.

A single binding region was identified on all three membranes. Binding of mAbs 1.1.2 and 1.5.3 was detected at peptides 27-30. The peptide representing the common epitope was determined to be: NPSEKNSPS (SEQ ID NO. 196). This peptide corresponds to residues 171-179 of the CD20 extracellular domain.

Epitope mapping for the 2.1.2 antibody was determined by flow cytometry using CHO cells expressing CD20 constructs with site directed mutations in the extracellular domain. Four CHO mutant lines were generated. A single binding region was identified for the three mAbs.

### Example 16

### CD20 Extracellular Region Sequence Alignment

Published reports indicate that antibodies directed against extracellular epitopes on human CD20 do not bind mouse B-cells. The extracellular domains of human and mouse CD20 differ in 16 of the approximate 43 amino acids. Eight non-conservative differences are located within a 10-amino acid stretch (ESLNFIRAHT (SEQ ID NO. 197)) as indicated in the alignment below.

TABLE 15
ALIGNMENT OF HUMAN AND MOUSE CD20 EXTRACELLULAR
REGION

| | Amino acids 142-184 |
|---|---|
| Human | KISHFLKMESLNFIRAHTPYINIYNCEPANPSEKNSPSTQYCY (SEQ ID NO 198) |
| Mouse | TL------RR-EL-QTSK--VD--D---S-S-----------N (SEQ ID NO 199) |

These differences in the amino acid sequence of the murine CD20 extracellular domain were used as a basis to conduct the above epitope mapping study. A mutation strategy was designed in which extracellular residues in the human CD20 sequence that differ from those in the mouse were replaced with those from the equivalent positions in the murine sequence.

### Example 17

### Epitope Mapping Using Site Directed Mutagenesis

Epitope mapping studies using a mutagenesis approach have indicated that Alanine at position 170 and Proline at position 172 are involved in the recognition of human CD20 by known anti-CD20 antibodies. The binding of known antibodies B1, 2H7, 1F5 and Rituximab was abrogated by mutation of these residues to Serine. Binding of mAbs 2F2 to human CD20 is insensitive to the A×P mutations, and represents a CD20 epitope involving N163 and N166.

The epitope of mAbs 1.1.2 and 1.5.3 was mapped to residues 171-179 (NPSEKNSPS (SEQ ID NO. 196)). Specificity of these mAbs to the human sequence is through Proline 172. Alanine 170 is not required for binding as shown in Table 16 below.

TABLE 16
FINE SPECIFICITY OF CD20 MABS

| | | | SEQ ID | |
|---|---|---|---|---|
| mAb | Specificity | SPOTS | NO. | Critical Residues |
| B1 | Human CD20 | PANPSEKNSP | 200 | A170, P172 |
| 2H7 | Human CD20 | | | A170*, P172 |
| IF5 | Human CD20 | | | A170*, P172 |
| Rituximab | Human CD20 | | | A170, P172 |
| 2F2 | Human CD20 | | | N163, N166 |
| 1.1.2 | Human CD20 | NPSEKNSPS | 196 | P172 |
| 1.5.3 | Human CD20 | NPSEKNSPS | 196 | P172 |
| 2.1.2 | Human CD20 | | | |

*Minimum requirement for binding. Polyak & Deans (2002); Blood 99: 3256-62.

Xenouse® derived anti-CD20 monoclonal antibodies as described herein have overlapping but different epitopes of all known CD20 antibodies.

### Example 18

### Uses of Anti-Cd20 Antibodies for the Treatment of Diseases Involving Expression of CD20

The lead anti-CD20 antibody candidates were evaluated in a Ramos i.v. rear-limb paralysis model. Cragg M S, Glennie, M J (2004) Blood 103:2738-43. More specifically, CB17 SCID mice were injected with 1×10⁶ human Ramos lymphoma cells via the tail vein and evaluated for onset of rear-limb paralysis and survival. Cohorts of animals were treated with the three lead candidate anti-CD20 antibodies; a cohort treated with Rituximab was established as a benchmark control. Thus, six cohorts of seven mice each were treated i.p. (intraperitoneal injection) with a single dose of 0.05 mg/kg of antibody fifteen days post tumor cell inoculation. The six cohorts were as follows: PBS (vehicle) control, IgG1 isotype control antibody, Rituximab, and the anti-CD20 antibodies 2.1.2, 1.3.3, and 1.1.2. Median and overall survival endpoints were monitored. Note: the sequence of anti-CD20 mAb 1.3.3 was found to be identical to that of mAb 1.5.3. For availability reasons, mAb 1.3.3 was used as a substitute for mAb 1.5.3.

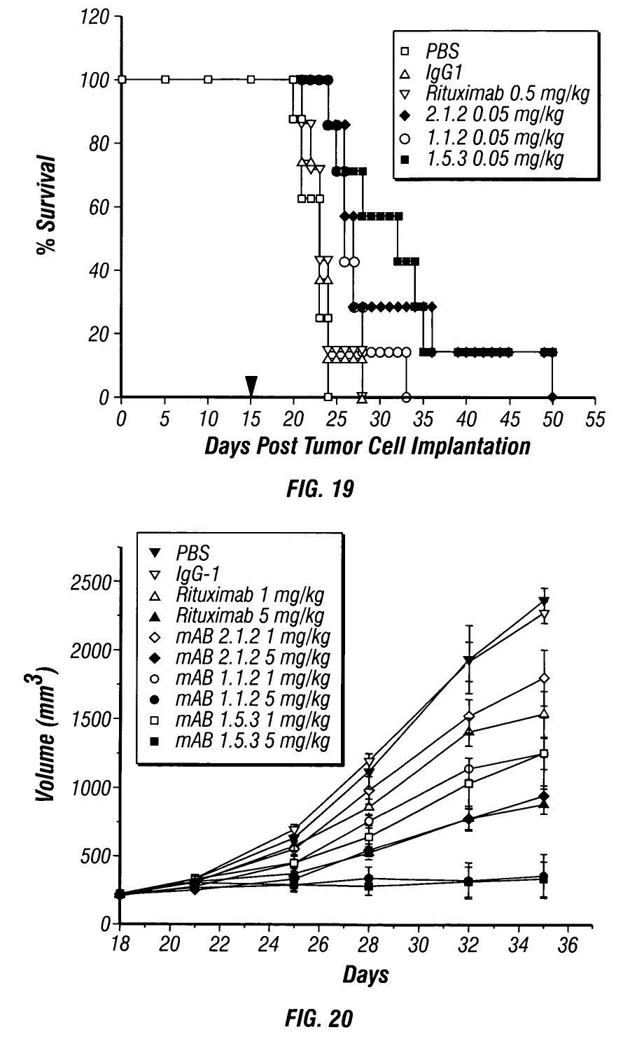

The results of the above study are shown in FIG. 19 and demonstrate that all three lead antibody candidates demonstrated potent anti-lymphoma activity when administered as a single dose monotherapy. Moreover, this study revealed that the overall survival of cohorts treated with the 1.5.3 and 2.1.2 antibodies was significantly improved as compared to the Rituximab control. Statistical analysis comparing the 1.5.3 antibody to Rituximab yielded a p-value of 0.022; analysis comparing the 2.1.2 to Rituximab yielded a p-value of 0.023. Therefore, these findings demonstrate a statistically significant improvement in overall survival in mice treated with the 1.5.3 and 2.1.2 anti-CD20 antibodies.

### Example 19

### Immunotherapy Using Human Antibodies Against CD20 in Subcutaneous Tumor Models

Efficacy in Daudi Subcutaneous Model

Immunotherapy with mAb 1.5.3 and Rituximab was evaluated in CB17 SCID mice with Daudi (ATCC) tumor cells. Briefly, CB17 SCID mice were obtained from Charles River laboratories, Wilmington, Mass., USA and maintained under pathogen free conditions. 107 Daudi cells were injected subcutaneously and allowed to form tumors. Treatments were initiated when the average tumor size reached 200 mm³. Each antibody, 2.1.2, 1.1.2, 1.5.3 and Rituximab, was tested at 2 dose levels, 1 mg/kg and 5 mg/kg, and compared to vehicle control and an IgG1 isotype control. Dosing of anti-CD20 antibodies, isotype control and PBS vehicle control was done by intraperitoneal injection twice a week for 3 weeks and was initiated at day 18 after tumor inoculation.

The results of the above study are shown in FIG. 20 and Table 17 below. All mAbs demonstrated potent antitumor efficacy. mAbs 1.5.3 and 1.1.2 were the more potent at inhibiting growth of Daudi tumors, with 95% (p<0.001) tumor growth inhibition at the 5 mg/kg dose relative to 71% inhibition for Rituximab. Statistical analysis comparing 1.5.3 or 1.1.2 to Rituximab yielded a p value below 0.05 in a Student t-test, indicating a significant improvement in efficacy with mAbs 1.1.2 and 1.5.3. Complete regression and tumor free survival was observed in 10% and 20% of mice treated with the higher dose of mAbs 1.5.3 and 1.1.2, respectively.

TABLE 17

| | Tumor | | Log | | | | | | | | |
|---|---|---|---|---|---|---|---|---|---|---|---|
| | Doubling | Growth | Cell | | | | | Max | Regression | Complete | Tumor |
| | Time | Delay | Kill | | t-Test | | S.D. | Weight | % | Regression % | Free |
| Group | (TD) | (GD) | (LCK) | T/C % | One-tail P | Inhibition % | log(Vf/Vi) | Loss % | [T/C <= | [Size <= | Survival |
| Assignments | [All] | [1000.0 mm³] | | [Start Day 18 End Day 35] | | | | | −50.0%] | 63.0 mm³] | [Days >= 14] |
| PBS | 4.607 | — | — | — | — | — | 0.08463 | 0% | — | — | — |
| IgG1-ctr | — | −0.4 | −0.029 | 95 | 0.21909 | 13 | 0.06266 | 0% | 0% | 0% | 0% |
| Rituxan- | — | 1.8 | 0.115 | 61 | 0.001727 | 46 | 0.14526 | 0% | 0% | 0% | 0% |
| 1 mpk | | | | | | | | | | | |
| Rituxan- | — | 9.0 | 0.591 | 31 | 0.0 | 71 | 0.10263 | 0% | 0% | 0% | 0% |
| 5 mpk | | | | | | | | | | | |
| 2.1.2-1 mpk | — | 0.9 | 0.057 | 73 | 0.037602 | 36 | 0.15279 | −1% | 0% | 0% | 0% |
| 2.1.2-5 mpk | — | 8.4 | 0.547 | 34 | 0.0 | 70 | 0.11548 | −1% | 0% | 0% | 0% |
| 1.1.2-1 mpk | — | 3.3 | 0.215 | 48 | 2.0E−6 | 55 | 0.15718 | 0% | 0% | 0% | 0% |
| 1.1.2-5 mpk | — | — | — | 6 | 0 | 95 | 0 | 0% | 30% | 20% | 20% |
| 1.5.3-1 mpk | — | 4.4 | 0.288 | 47 | 8.14E−4 | 59 | 0.24263 | 0% | 0/10 | 0% | 0% |
| 1.5.3-5 mpk | — | 24.6 | 1.608 | 5 | 0.0 | 95 | 0 | −2% | 10% | 10% | 10% |

Efficacy in Namalwa Subcutaneous Model

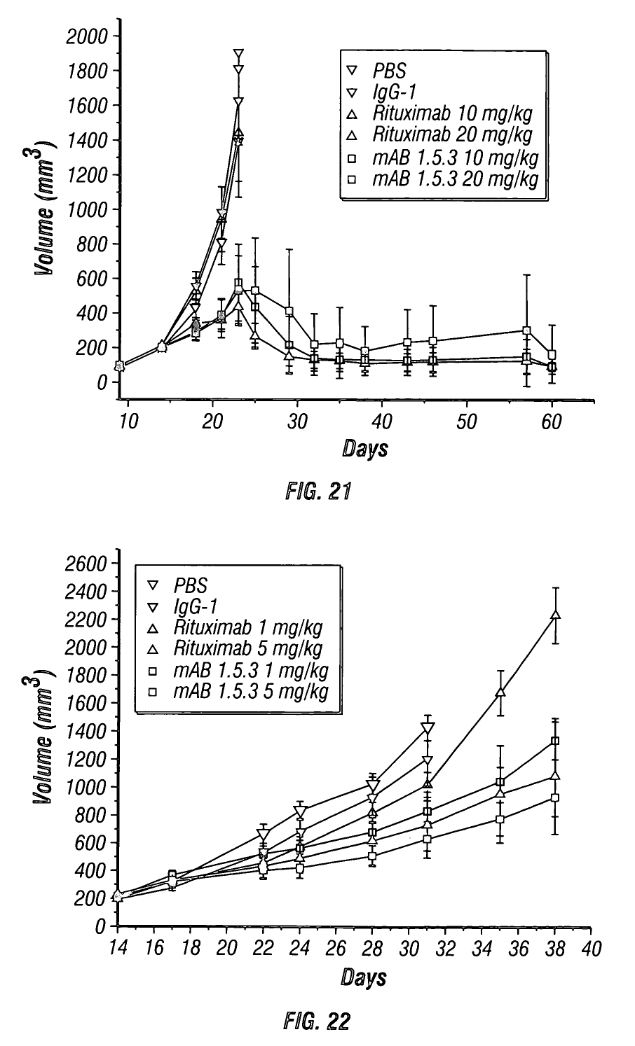

A similar experimental design was used to evaluate the efficacy of anti-CD20 human antibody in the Namalwa model of Non-Hodgkin lymphoma. 107 Namalwa (ATCC) cells were implanted subcutaneously in Ncr nude mice (Taconics, Germantown, N.Y., USA). Namalwa cells formed aggressive tumors expressing low level of CD20. Treatment was initiated when the tumors reached an average size of 100 mm³. Antibodies were tested at dose levels of 10 and 20 mg/kg of anti-CD20 antibodies, and compared to vehicle control and an IgG1 isotype control. Dosing of anti-CD20 antibodies, isotype control and PBS vehicle control was done by intraperitoneal injection twice a week for 3 weeks and was initiated at day 9 after tumor inoculation. As shown in FIG. 21 and Table 18 below, Rituximab and mAb 1.5.3 are equivalent at the highest dose of 20 mg/kg mediating a tumor growth inhibition of 78 and 73% (p<0.001) respectively. However at a dose of 10 mg/kg 1.5.3 was dramatically more potent than the same dose of Rituximab. Rituximab was not efficacious, while mAb 1.5.3 still displayed 65% tumor growth inhibition (p<0.05).

TABLE 18

| | Tumor | | Log | | | | | | | |
|---|---|---|---|---|---|---|---|---|---|---|
| | Doubling | Growth | Cell | | | | S.D. | Max | | |
| | Time | Delay | Kill | T/C | t-Test | Inhibition | log(Vf/ | Weight | | Complete |
| Group | (TD) | (GD) | (LCK) | % | One-tail P | % | Vi) | Loss % | Regression | Regression |
| Assignments | [All] | [1000.0 mm³] | | [Start Day 9 End Day 23] | | | | | [T/C <= −50.0%] | [Size <= 63.0 mm³] |
| PBS | 3.399 | — | — | — | — | — | NaN | 0% | 0/10 | 0/10 |
| IgG-1 | — | 0.6 | 0.053 | 85 | 0.212797 | 2 | NaN | 0% | 0% | 0% |
| RTX-10 mpk | — | 0.2 | 0.015 | 88 | 0.498394 | 5 | NaN | 0% | 0% | 0% |
| RTX-20 mpk | — | N/A | N/A | 22 | 5.76E−4 | 78 | NaN | 0% | 80% | 80% |
| 1.5.3-10 mpk | — | N/A | N/A | 32 | 0.023883 | 65 | NaN | 0% | 40% | 40% |
| 1.5.3-20 mpk | — | N/A | N/A | 28 | 0.005959 | 73 | 0.44054 | 0% | 40% | 30% |

Efficacy in RR1-Raji Subcutaneous Model

The efficacy of anti-CD20 human antibodies in a Rituximab-resistant cell model was also evaluated. 107 RR1-Raji were implanted subcutaneously in CB17 SCID recipient mice. Antibodies were tested at two dose levels, 1 mg/kg and 5 mg/kg, and compared to vehicle control and an IgG1 isotype control. Dosing of anti-CD20 antibodies, isotype control and PBS vehicle control was done by intraperitoneal injection twice a week for 3 weeks and was initiated at day 14 after tumor inoculation. Rituximab at a concentration of 5 mg/kg was efficacious in RR1-Raji model mediating 59% tumor growth inhibition similar to the 62% achieved by mAb 1.5.3 (FIG. 22). At 1 mg/kg weekly dose, antibody 1.5.3 appeared more potent than Rituximab, although the difference did not reach statistical significance (p=0.058).

TABLE 19

| | Tumor | | | | | |
|---|---|---|---|---|---|---|
| | Doubling | Growth | Log Cell | | t-Test | |
| | Time | Delay | Kill | | One-tail P | |
| Group | (TD) | (GD) | (LCK) | T/C % | Show Chart | Inhibition % |
| Assignments | [All] | [900.0 mm³] | | [Start Day 14 End] | | |
| PBS | 6.334 | — | — | — | — | — |
| IgG1 @ 5 mpk | — | −2.1 | −0.1 | 120 | 0.249902 | −9 |
| Rituxan @ 1 mpk | — | 1.7 | 0.082 | 81 | 0.086581 | 20 |
| Rituxan @ 5 mpk | — | 6.6 | 0.3 13 | 49 | 0.021599 | 59 |
| 1.5.3 @ 1 mpk | — | 4.8 | 0.229 | 61 | 0.009539 | 45 |
| 1.5.3 @ 5 mpk | — | 9.9 | 0.473 | 42 | 9.18E−4 | 62 |

Efficacy in RR6-Ramos Subcutaneous Model

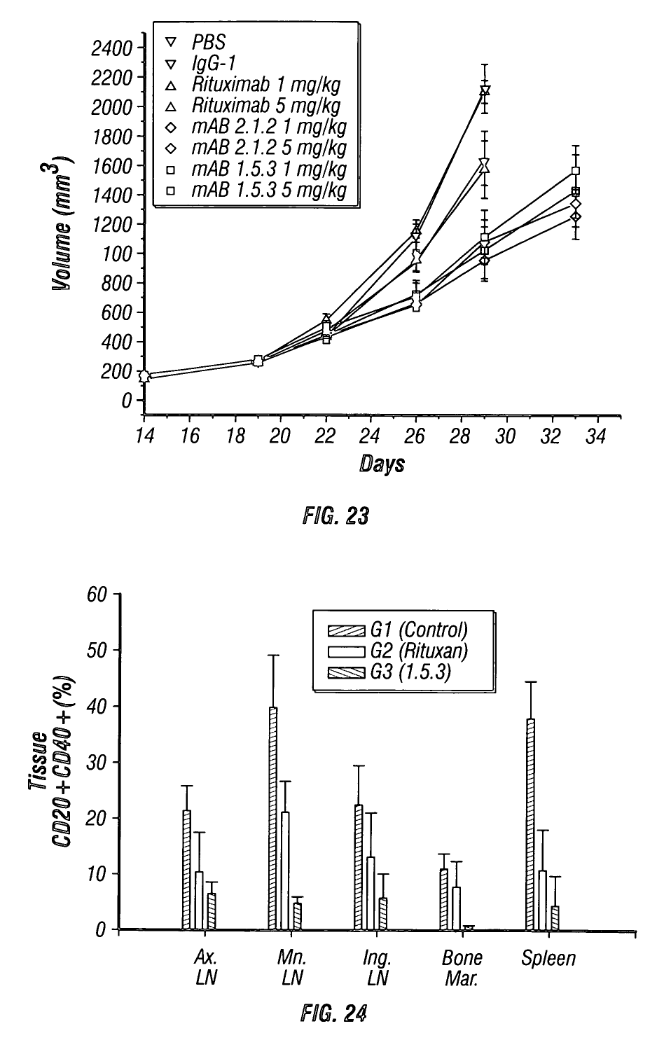

The RR6-Ramos model was then tried in vivo. 107 RR6-Ramos cells were implanted subcutaneously in CB17 SCID recipient mice. The mice were treated with 1 or 5 mg/kg of 2.1.2, 1.5.3 or Rituximab and the antitumor efficacy was compared to vehicle PBS control or an IgG1 isotype control. Dosing of anti-CD20 antibodies, isotype control and PBS vehicle control was done by intraperitoneal injection twice a week for 3 weeks and was initiated at day 14 after tumor inoculation. FIG. 23 and Table 20 demonstrate that mAbs 2.1.2 and 1.5.3 inhibited tumor growth by 61% and 59% (p<0.05) respectively while Rituximab did not mediate any significant antitumor effect in this model.

TABLE 20

| | Tumor | | | | | |
|---|---|---|---|---|---|---|
| | Doubling | Growth | Log Cell | | | |
| | Time | Delay | Kill | | t-Test | |
| Group | (TD) | (GD) | (LCK) | T/C % | One-tail P | Inhibition % |
| Assignments | [All] | [1000.0 mm³] | | [Start Day 14 End] | | |
| IgG1 @ 5 mpk | 3.823 | — | — | — | — | — |
| Rituxan @ 1 mpk | — | −0.4 | −0.029 | 97 | 0.382272 | 17 |
| Rituxan @ 5 mpk | — | 0.9 | −0.068 | 72 | 0.019239 | 26 |
| 2.1.2 @ 1 mpk | — | 4.3 | 0.337 | 40 | 2.05E−5 | 64 |
| 2.1.2 @ 1 mpk | — | 3.1 | 0.244 | 46 | 1.5E−5 | 61 |
| 1.5.3 @ 1 mpk | — | 3.4 | 0.27 | 44 | 7.53E−4 | 59 |
| 1.5.3 @ 5 mpk | — | 2.8 | 0.221 | 48 | 6.05E−4 | 59 |

### Example 20

### Depletion of Tissue B-Cells Following Treatment with Human Antibodies Against CD20

The degree of amino acid idenity/homology between cynomolgus monkey CD20 and human CD20 is presumably high as many commercially available anti-human CD20 antibodies cross-react with cynomolgus monkey B-lymphocytes. A total of twenty-six male cynomolgus monkeys were screened, prior to the start of the study, for the proportion of circulating B lymphocytes (CD20+ cells). Animals showing extreme/unusual prevalence of CD20+ cells on either side of the subpopulation distribution were excluded. Twenty-four animals were selected as a result of the screening process, randomized by body weight, and assigned to three groups each consisting of six male cynomolgus monkeys. Following a 14-day acclimation period, groups were treated intravenously, on Study Days 1, 8, and 15, with vehicle (Group 1, 0 mg/kg), Rituximab (Group 2, 10 mg/kg), as a positive control, and mAb 1.5.3, 10 mg/kg.

Parameters evaluated included clinical observations, body weight, food consumption, hematology, FACS analysis of peripheral blood and lymphoid tissues, test article concentrations in serum (pharmacokinetics), gross pathology, organ weights, histopathology and immunohistochemistry. All animals survived until the scheduled terminal necropsy on Study Day 18.

No changes attributable to the positive control (Rituximab) or the test article (mAb 1.5.3) treatment were apparent in clinical observations, food consumption, body weight, organ weights or gross pathology. As expected, there were marked test article-related effects on total lymphocyte counts in blood as early as 1 hour post-infusion as well as other expected changes in hematology. Flow cytometry results demonstrated a profound depletion in relative percentages of positive B lymphocytes relative to baseline in CD19+ and CD20+ subsets for Group 2 (Rituximab), and Group 3 (mAb 1.5.3) at equivalent doses in blood. mAb 1.5.3 treated monkeys showed the most pronounced effects occurring immediately post-dosing with depleted B-cell levels remaining at approximately >1% of the baseline at the end of the study on Day 18.

Additionally, results from the FACS analyses of cells isolated from lymph nodes from various sites (mesenteric, inguinal, and auxiliary lymph nodes), bone marrow and spleen demonstrated the greatest differences (P<0.05) between Group 3, mAb 1.5.3 treated animals, and the control group (FIG. 24). The effects observed with mAb 1.5.3 were more pronounced than those observed with Rituximab at equivalent doses (FIG. 24). These changes included minimal to marked B-cell depletions within follicles and the marginal zone (spleen) and relative and/or absolute increases in T-cells in the periarteriolar lymphoid sheaths (PALS) and paracortex. Overall, no adverse findings attributed to test article administration were noted in this study. All test-article related changes observed in the study animals were consistent with the pharmacologic activity of anti-CD20+ antibodies with antibody AB1 demonstrating the most potent effects.

### Example 21

Human patients with Non-Hodgkin's Lymphoma are treated with mAb 1.5.3 described herein. Each patient is dosed weekly with an effective amount of the antibody ranging from 50 mg/m² to 2,250 mg/m² for 4-8 weeks. At periodic times during the treatment, each patient is monitored to determine the number of lymphoma cells in the patient. It is found that in patients undergoing treatment with the mAb 1.5.3, the number of lymphoma cells is reduced in comparison to control patients that are not given the mAb 1.5.3 antibody.

### INCORPORATION BY REFERENCE

All references cited herein, including patents, patent applications, papers, text books, and the like, and the references cited therein, to the extent that they are not already, are hereby incorporated herein by reference in their entirety.

### EQUIVALENTS

The foregoing written specification is considered to be sufficient to enable one skilled in the art to practice the invention. The foregoing description and Examples detail certain preferred embodiments of the invention and describes the best mode contemplated by the inventors. It will be appreciated, however, that no matter how detailed the foregoing may appear in text, the invention can be practiced in many ways and the invention should be construed in accordance with the appended claims and any equivalents thereof.

What is claimed is:

1. A targeted binding agent that binds CD20 and comprises a heavy chain complementarity determining region 1 (CDR1) having an amino acid sequence of GYSFTSYWIG (SEQ ID NO.: 201).

2. A targeted binding agent that binds CD20 and comprises a light chain complementarity determining region 2 (CDR2) having an amino acid sequence of KISNRFS (SEQ ID NO.: 202).

3. A targeted binding agent that binds CD20, wherein the targeted binding agent has an EC₅₀ of no more than 0.5 μg/ml for inducing apoptosis of Ramos cells in a standard CellTiterGlo viability assay.

4. The targeted binding agent of claim 3, wherein the targeted binding agent has an EC₅₀ of no more than 0.2 μg/ml for inducing apoptosis of Ramos cells in a standard CellTiterGlo viability assay.

5. The targeted binding agent of claim 3, wherein the targeted binding agent has an EC₅₀ of no more than 0.02 μg/ml for inducing apoptosis of Ramos cells in a standard CellTiterGlo viability assay.

6. A targeted binding agent that binds CD20, wherein the targeted binding agent has an EC₅₀ of no more than 0.2 μg/ml for inducing apoptosis of Ramos cells in a standard Alamar Blue viability assay.

7. The targeted binding agent of claim 6, wherein the targeted binding agent has an EC₅₀ of no more than 0.09 μg/ml for inducing apoptosis of Ramos cells in a standard Alamar Blue viability assay.

8. The targeted binding agent of claim 6, wherein the targeted binding agent has an EC₅₀ of no more than 0.04 μg/ml for inducing apoptosis of Ramos cells in a standard Alamar Blue viability assay.

9. The targeted binding agent of claim 1, wherein said targeted binding agent induces apoptosis in cells expressing CD20, induces antibody dependent cellular cytotoxicity (ADCC) in cells expressing CD20, or induces complement dependent cytotoxicity (CDC) in cells expressing CD20.

10. The targeted binding agent of claim 1, wherein said targeted binding agent binds to CD20 with a Kd of less than 12 nanomolar (nM).

11. The targeted binding agent of claim 1, wherein said targeted binding agent is monoclonal antibody 1.1.2 (ATCC Accession Number PTA-7329).

12. The targeted binding agent of claim 1, wherein said targeted binding agent is monoclonal antibody 1.5.3 (ATCC Accession Number PTA-7330).

13. The targeted binding agent of claim 1, wherein said targeted binding agent is monoclonal antibody 2.1.2 (ATCC Accession Number PTA-7328).

14. The targeted binding agent of claim 1, wherein the targeted binding agent comprises a heavy chain polypeptide having the sequence of SEQ ID NO.: 2.

15. The targeted binding agent of claim 14, wherein the targeted binding agent comprises a light chain polypeptide having the sequence of SEQ ID NO.: 4.

16. The targeted binding agent of claim 1, wherein the targeted binding agent comprises a heavy chain polypeptide having the sequence of SEQ ID NO.: 30.

17. The targeted binding agent of claim 16, wherein the targeted binding agent comprises a light chain polypeptide having the sequence of SEQ ID NO.: 32.

18. The targeted binding agent of claim 1, wherein the targeted binding agent comprises a heavy chain polypeptide having the sequence of SEQ ID NO.: 46.

19. The targeted binding agent of claim 18, wherein the targeted binding agent comprises a light chain polypeptide having the sequence of SEQ ID NO.: 48.

20. The targeted binding agent of claim 1 in association with a pharmaceutically acceptable carrier.

21. A nucleic acid molecule encoding the targeted binding agent of claim 1.

22. A vector comprising the nucleic acid molecule of claim 21.

23. A host cell comprising the vector of claim 22.

24. A method of treating a B-cell lymphoma in an animal, comprising administering to said animal in need thereof a therapeutically effective dose of the targeted binding agent of claim 1.

25. The method of claim 24, wherein said B-cell lymphoma is non-Hodgkin's lymphoma (NHL).

26. The method of claim 24, wherein said animal is human.

27. The method of claim 24, wherein said targeted binding agent is the mAb 1.1.2 (ATCC Accession Number PTA-7329) or mAb 1.5.3 (ATCC Accession Number PTA-7330) or mAb 2.1.2 (ATCC Accession Number PTA-7328).

28. The method of claim 24, further comprising administering a second agent selected from the group consisting of an antibody, a chemotherapeutic drug, and a radioactive drug.

29. The method of claim 24, wherein said administering is in conjunction with or following a conventional surgery, a bone marrow stem cell transplantation or a peripheral stem cell transplantation.
